SEQUENCE LISTING (132 sequences)

SEQ ID NO: 1 - DNA, 38 bp, Artificial Sequence
  PCR Primer
tttttttgcattaatctctagaggatccccagttgggt

SEQ ID NO: 2 - DNA, 38 bp, Artificial Sequence
  PCR Primer
tttttttagctaaatttgattcccaattctctgaatat

SEQ ID NO: 3 - DNA, 38 bp, Artificial Sequence
  PCR Primer
  modified_base (1)..(1): dual biotinylated base
ttttttttataacagcggttgtaccaaaaagcctttat

SEQ ID NO: 4 - DNA, 38 bp, Artificial Sequence
  PCR Primer
  modified_base (1)..(1): dual biotinylated base
tttttttaactgaccgtaatgccactacgacgagggta

SEQ ID NO: 5 - DNA, 39 bp, Artificial Sequence
  PCR Primer
  modified_base (1)..(1): dual biotinylated base
tttttttcgcgacctcatctttgacccccaggcagggag

SEQ ID NO: 6 - DNA, 38 bp, Artificial Sequence
  PCR Primer
  modified_base (1)..(1): dual biotinylated base
tttttttcgccaccaaggaggttgaggcagaaacatga

SEQ ID NO: 7 - DNA, 38 bp, Artificial Sequence
  PCR Primer
  modified_base (1)..(1): dual biotinylated base
tttttttaccgcctcgccagaatggaaagctgagtaac

SEQ ID NO: 8 - DNA, 38 bp, Artificial Sequence; identical to SEQ ID NO: 6
  PCR Primer
  modified_base (1)..(1): dual biotinylated base

SEQ ID NO: 9 - DNA, 38 bp, Artificial Sequence
  PCR Primer
  modified_base (1)..(1): dual biotinylated base
tttttttactcacattgaaattgttatccgcggtgcgg

SEQ ID NO: 10 - DNA, 38 bp, Artificial Sequence
  PCR Primer
  modified_base (1)..(1): dual biotinylated base
tttttttgaattatctcatcaatataatcctttcaatt

SEQ ID NO: 11 - DNA, 38 bp, Artificial Sequence
  PCR Primer
  modified_base (1)..(1): dual biotinylated base
tttttttttaattttcatatcaaaattatttaacggat

SEQ ID NO: 12 - DNA, 89 bp, Artificial Sequence
  PCR Primer
  modified_base (1)..(1): biotinylated base
  modified_base (10)..(10): CNV-K phosphoramidite
  misc_feature (10)..(10): n is a, c, g, or t
ttttttgcangcgtttttttttttttttttttttttttttttttacgcgtgcatttttaccgcctcgccagaatggaaagctgagtaac

SEQ ID NO: 13 - DNA, 61 bp, Artificial Sequence
  PCR Primer
ggaacttcagcccaactaacattttaccgccacttttatgatacaggagtgtatcatacat

SEQ ID NO: 14 - DNA, 61 bp, Artificial Sequence
  PCR Primer
ggaacttcagcccaactaacattttggtttaacttttattagactttacaaacttgaggat

SEQ ID NO: 15 - DNA, 38 bp, Artificial Sequence; identical to SEQ ID NO: 10
  PCR Primer
  modified_base (1)..(1): dual biotinylated base

SEQ ID NO: 16 - DNA, 15 bp, Artificial Sequence
  PCR Primer
  modified_base (1)..(1): dual biotinylated base
gtggcgagcccgatc

SEQ ID NO: 17 - DNA, 19 bp, Artificial Sequence
  PCR Primer
gctagttattgctcagcgg

SEQ ID NO: 18 - DNA, 13 bp, Artificial Sequence
  PCR Primer
gccggtgatgccg

SEQ ID NO: 19 - DNA, 16 bp, Artificial Sequence
  PCR Primer
  modified_base (1)..(1): dual biotinylated base
ctgcgcgtaaccacca

SEQ ID NO: 20 - DNA, 13 bp, Artificial Sequence
  PCR Primer
  modified_base (1)..(1): dual biotinylated base
gccggtgatgccg

SEQ ID NO: 21 - DNA, 17 bp, Artificial Sequence
  PCR Primer
  modified_base (1)..(1): dual biotinylated base
aacagtcccccggccac

SEQ ID NO: 22 - DNA, 15 bp, Artificial Sequence
  PCR Primer
gtggcgagcccgatc

SEQ ID NO: 23 - DNA, 45 bp, Artificial Sequence
  PCR Primer
gccggtgatgccgatcccgcgaaattaatacgactcactataggg

SEQ ID NO: 24 - DNA, 38 bp, Artificial Sequence
  PCR Primer
gccggtgatgccggaggatcgagatcgatctcgatccc

SEQ ID NO: 25 - DNA, 50 bp, Artificial Sequence
  PCR Primer
gccggtgatgccgccagcaaccgcacctgtggcgccacgatgcgtccggc

SEQ ID NO: 26 - DNA, 10 bp, Artificial Sequence
  PCR Primer
  modified_base (1)..(1): dual biotinylated base
tttttttttt

SEQ ID NO: 27 - DNA, 15 bp, Artificial Sequence
  PCR Primer
  modified_base (1)..(1): dual biotinylated base
ttttttttttttttt

SEQ ID NO: 28 - DNA, 20 bp, Artificial Sequence
  PCR Primer
  modified_base (1)..(1): dual biotinylated base
tttttttttttttttttttt

SEQ ID NO: 29 - DNA, 30 bp, Artificial Sequence
  PCR Primer
  modified_base (1)..(1): dual biotinylated base
tttttttttttttttttttttttttttttt

SEQ ID NO: 30 - DNA, 40 bp, Artificial Sequence
  PCR Primer
  modified_base (1)..(1): dual biotinylated base
tttttttttttttttttttttttttttttttttttttttt

SEQ ID NO: 31 - DNA, 50 bp, Artificial Sequence
  PCR Primer
  modified_base (1)..(1): dual biotinylated base
tttttttttttttttttttttttttttttttttttttttttttttttttt

SEQ ID NO: 32 - DNA, 60 bp, Artificial Sequence
  PCR Primer
  modified_base (1)..(1): dual biotinylated base
tttttttttttttttttttttttttttttttttttttttttttttttttttttttttttt

SEQ ID NO: 33 - DNA, 59 bp, Artificial Sequence
  PCR Primer
  modified_base (1)..(1): dual biotinylated base
tcttcactatacaacctactacctcattttttttttttttttttgtaggttgtatagtg

SEQ ID NO: 34 - DNA, 59 bp, Artificial Sequence
  PCR Primer
  modified_base (1)..(1): dual biotinylated base
tcttcactatacaacctactacctcattttttttttttttttttttaggttgtatagtg

SEQ ID NO: 35 - DNA, 59 bp, Artificial Sequence
  PCR Primer
  modified_base (1)..(1): dual biotinylated base
tcttcactatacaacctactacctcatttttttttttttttttttaaggttgtatagtg

SEQ ID NO: 36 - DNA, 59 bp, Artificial Sequence
  PCR Primer
  modified_base (1)..(1): dual biotinylated base
tcttcactatacaacctactacctcatttttttttttttttttttttggttgtatagtg

SEQ ID NO: 37 - DNA, 59 bp, Artificial Sequence
  PCR Primer
  modified_base (1)..(1): dual biotinylated base
tcttcactatacaacctactacctcattttttttttttttttttttttgttgtatagtg

SEQ ID NO: 38 - DNA, 59 bp, Artificial Sequence
  PCR Primer
  modified_base (1)..(1): dual biotinylated base
tcttcactatacaacctactacctcatttttttttttttttttttttttttgtatagtg

SEQ ID NO: 39 - DNA, 59 bp, Artificial Sequence
  PCR Primer
  modified_base (1)..(1): dual biotinylated base
tcttcactatacaacctactacctcatttttttttttttttttttttttatgtatagtg

SEQ ID NO: 40 - DNA, 59 bp, Artificial Sequence
  PCR Primer
  modified_base (1)..(1): dual biotinylated base
tcttcactatacaacctactacctcatttttttttttttttttttttttaagtatagtg

SEQ ID NO: 41 - DNA, 59 bp, Artificial Sequence
  PCR Primer
  modified_base (1)..(1): dual biotinylated base
tcttcactatacaacctactacctcatttttttttttttttttttttttttttatagtg

SEQ ID NO: 42 - DNA, 59 bp, Artificial Sequence
  PCR Primer
  modified_base (1)..(1): dual biotinylated base
tcttcactatacaacctactacctcattttttttttttttttttttttttttaatagtg

SEQ ID NO: 43 - DNA, 59 bp, Artificial Sequence
  PCR Primer
  modified_base (1)..(1): dual biotinylated base
tcttcactatacaacctactacctcatttttttttttttttttttttttttttttagtg

SEQ ID NO: 44 - DNA, 59 bp, Artificial Sequence
  PCR Primer
  modified_base (1)..(1): dual biotinylated base
tctgcactatacaacctactacctcattttttttttttttttttttggttgtatagtgc

SEQ ID NO: 45 - DNA, 59 bp, Artificial Sequence
  PCR Primer
  modified_base (1)..(1): dual biotinylated base
tccgcactatacaacctactacctcatttttttttttttttttttggttgtatagtgcg

SEQ ID NO: 46 - DNA, 59 bp, Artificial Sequence
  PCR Primer
  modified_base (1)..(1): dual biotinylated base
tccgcactatacaacctactacctcattttttttttttttttttggttgtatagtgcgg

SEQ ID NO: 47 - DNA, 59 bp, Artificial Sequence
  PCR Primer
  modified_base (1)..(1): dual biotinylated base
tcttcatacatacttctttacattccattttttttttttttttttaagaagtatgtatg

SEQ ID NO: 48 - DNA, 59 bp, Artificial Sequence
  PCR Primer
  modified_base (1)..(1): dual biotinylated base
tcttcccacacacttccttacattccatttttttttttttttttttttaagtgtgtggg

SEQ ID NO: 49 - DNA, 59 bp, Artificial Sequence
  PCR Primer
  modified_base (1)..(1): dual biotinylated base
tcttcacaggccgggacaagtgcaatatttttttttttttttttttttttcggcctgtg

SEQ ID NO: 50 - DNA, 59 bp, Artificial Sequence
  PCR Primer
  modified_base (1)..(1): dual biotinylated base
tcttcgctgggtggagaaggtggtgaatttttttttttttttttttttttcacccagcg

SEQ ID NO: 51 - DNA, 59 bp, Artificial Sequence
  PCR Primer
  modified_base (1)..(1): dual biotinylated base
tcttcaacggaaccactagtgacttgttttttttttttttttttttttaggttccgttg

SEQ ID NO: 52 - DNA, 59 bp, Artificial Sequence
  PCR Primer
  modified_base (1)..(1): dual biotinylated base
tcttcataaggatttttaggggcattattttttttttttttttttaaaaaatccttatg

SEQ ID NO: 53 - DNA, 59 bp, Artificial Sequence
  PCR Primer
  modified_base (1)..(1): dual biotinylated base
tcttcagtgaattctaccagtgccatatttttttttttttttttttgtagaattcactg

SEQ ID NO: 54 - DNA, 87 bp, Artificial Sequence
  PCR Primer
  modified_base (1)..(1): dual biotinylated base
actatacaacctttttttttttttttttttttttttttttttttttttttttttttttttttttttttttttttttttactacctca

SEQ ID NO: 55 - DNA, 77 bp, Artificial Sequence
  PCR Primer
  modified_base (1)..(1): dual biotinylated base
actatacaacccgccttttttttttttttttttttttttttttttttttttttttttttttttggcgtactacctca

SEQ ID NO: 56 - DNA, 77 bp, Artificial Sequence
  PCR Primer
  modified_base (1)..(1): dual biotinylated base
actatacaaccccgcccttttttttttttttttttttttttttttttttttttttttttttgggcggtactacctca

SEQ ID NO: 57 - DNA, 77 bp, Artificial Sequence
  PCR Primer
  modified_base (1)..(1): dual biotinylated base
actatacaaccccgccccttttttttttttttttttttttttttttttttttttttttttggggcggtactacctca

SEQ ID NO: 58 - DNA, 77 bp, Artificial Sequence
  PCR Primer
  modified_base (1)..(1): dual biotinylated base
actatacaacccccgtcccccttttttttttttttttttttttttttttttttttttgggggacgggtactacctca

SEQ ID NO: 59 - DNA, 47 bp, Artificial Sequence
  PCR Primer
  modified_base (1)..(1): dual biotinylated base
gccggtgatgccgcgaaattaatacgactcactataggggaattgtg

SEQ ID NO: 60 - DNA, 87 bp, Artificial Sequence
  PCR Primer
  modified_base (1)..(1): dual biotinylated base
tcactatacaacctactacctcatttttttttttggttgtatagtgtgctgggtggagaaggtggtgaattttttttaccacccagc

SEQ ID NO: 61 - DNA, 133 bp, Artificial Sequence
  PCR Primer
  modified_base (1)..(1): dual biotinylated base
tcactatacaacctactacctcatttttttttttggttgtatagtgtcccacacacttccttacattccatttttttttttaagtgtgtgggtcacaggccgggacaagtgcaatattttttttcggcctgtg

SEQ ID NO: 62 - DNA, 133 bp, Artificial Sequence
  PCR Primer
  modified_base (1)..(1): dual biotinylated base
tcactatacaacctactacctcatttttttttttggttgtatagtgtcccacacacttccttacattccatttttttttttaagtgtgtgggtgctgggtggagaaggtggtgaattttttttaccacccagc

SEQ ID NO: 63 - DNA, 133 bp, Artificial Sequence
  PCR Primer
  modified_base (1)..(1): dual biotinylated base
tcactatacaacctactacctcatttttttttttggttgtatagtgtcataaggatttttaggggcattattatttaaaaaatccttatgtcagtgaattctaccagtgccatattttttgtagaattcactg

SEQ ID NO: 64 - DNA, 133 bp, Artificial Sequence; identical to SEQ ID NO: 61
  PCR Primer
  modified_base (1)..(1): dual biotinylated base

SEQ ID NO: 65 - DNA, 17 bp, Artificial Sequence
  PCR Primer
  modified_base (1)..(1): dual biotinylated base
gtggcgagcccgatctt

SEQ ID NO: 66 - DNA, 87 bp, Artificial Sequence
  PCR Primer
  modified_base (1)..(1): dual biotinylated base
tcccacacacttccttacattccatttttttttttaagtgtgtgggtcacaggccgggacaagtgcaatattttttttcggcctgtg

SEQ ID NO: 67 - DNA, 51 bp, Artificial Sequence
  PCR Primer
  modified_base (2)..(2): cm
actatacaacctactacctcacccctatagtgagtcgtattaatttcgcgg

SEQ ID NO: 68 - DNA, 38 bp, Artificial Sequence; identical to SEQ ID NO: 1
  PCR Primer
  modified_base (1)..(1): dual biotinylated base

SEQ ID NO: 69 - DNA, 15 bp, Artificial Sequence
  PCR Primer
  modified_base (1)..(1): dual biotinylated base
tccccatcggtgatg

SEQ ID NO: 70 - DNA, 52 bp, Artificial Sequence
  PCR Primer
  modified_base (2)..(2): cm
gctgggtggagaaggtggtgaacccctatagtgagtcgtattaatttcgcgg

SEQ ID NO: 71 - DNA, 63 bp, Artificial Sequence
  Synthetic Sequence - part of mCherry
atggtgagcaagggcgaggaggataacatggccatcatcaaggagttcatgcgcttcaaggtg

SEQ ID NO: 72 - DNA, 63 bp, Artificial Sequence
  Synthetic Sequence - part of optimized mCherry
  CDS (1)..(63)
atggtttctaaaggtgaagaagataacatggcaattattaaagaatttatgcgttttaaagtt

SEQ ID NO: 73 - protein, 21 aa, Artificial Sequence
  Synthetic Construct
MVSKGEEDNMAIIKEFMRFKV

SEQ ID NO: 74 - DNA, 28 bp, Artificial Sequence
  Synthetic Sequence - Complementary Strand
taagggaaaattaattaatagcgacgat

SEQ ID NO: 75 - DNA, 26 bp, Artificial Sequence
  Synthetic Sequence - Complementary Strand
tagcctttgtagatctctcaaaaata

SEQ ID NO: 76 - DNA, 20 bp, Artificial Sequence
  PCR Primer
taatagcgacgatttacaga

SEQ ID NO: 77 - DNA, 23 bp, Artificial Sequence
  PCR Primer
tacaaaggctatcaggtcattgc

SEQ ID NO: 78 - DNA, 31 bp, Artificial Sequence
  Synthetic Sequence - Staple DNA
aaagaatacagcggggtcattgctggaggtg

SEQ ID NO: 79 - DNA, 31 bp, Artificial Sequence
  Synthetic Sequence - Staple DNA
tggtgtgaatccgccgggcgcgggtggtgcc

SEQ ID NO: 80 - DNA, 32 bp, Artificial Sequence
  Synthetic Sequence - Staple DNA
gtttctttgtcactgttgccctgccaaccgca

SEQ ID NO: 81 - DNA, 24 bp, Artificial Sequence
  Synthetic Sequence - Staple DNA
tccagcattttttgggctagggcg

SEQ ID NO: 82 - DNA, 23 bp, Artificial Sequence
  Synthetic Sequence - Staple DNA
atcccacgcaaccagcatcctca

SEQ ID NO: 83 - DNA, 24 bp, Artificial Sequence
  Synthetic Sequence - Staple DNA
agaatgccaacggcagaacgtcag

SEQ ID NO: 84 - DNA, 31 bp, Artificial Sequence
  Synthetic Sequence - Staple DNA
ctggcaacccgccgcgcttaatggttgtgta

SEQ ID NO: 85 - DNA, 31 bp, Artificial Sequence
  Synthetic Sequence - Staple DNA
taacggaacctccggccagagcaaaaaaagc

SEQ ID NO: 86 - DNA, 31 bp, Artificial Sequence
  Synthetic Sequence - Staple DNA
cgtggtgcgcggtccgttttttcagggtaaa

SEQ ID NO: 87 - DNA, 38 bp, Artificial Sequence
  Synthetic Sequence - Staple DNA
  modified_base (1)..(1): aminated base
tttttttcatcgaccgccagcagttgggcgcctgagta

SEQ ID NO: 88 - DNA, 38 bp, Artificial Sequence
  Synthetic Sequence - Staple DNA
  modified_base (1)..(1): dual biotinylated base
tttttttcgcacagggcggatcaaacttaaggcgaaac

SEQ ID NO: 89 - DNA, 30 bp, Artificial Sequence
  Synthetic Sequence - Staple DNA
  modified_base (1)..(1): dual biotinylated base
tttttttgaagaacacaatattaccgccag

SEQ ID NO: 90 - DNA, 23 bp, Artificial Sequence
  Synthetic Sequence - Staple DNA
gtacagcgtcaccggaaacaatc

SEQ ID NO: 91 - DNA, 23 bp, Artificial Sequence
  Synthetic Sequence - Staple DNA
gtgagagcagaggtggagccgcc

SEQ ID NO: 92 - DNA, 31 bp, Artificial Sequence
  Synthetic Sequence - Staple DNA
ccattgcatcgctattacgccagatcggtgc

SEQ ID NO: 93 - DNA, 32 bp, Artificial Sequence
  Synthetic Sequence - Staple DNA
gctggtaagtgctgcaaggcgattcattcagg

SEQ ID NO: 94 - DNA, 31 bp, Artificial Sequence
  Synthetic Sequence - Staple DNA
acgggaacgggttttcccagtcatggtgccg

SEQ ID NO: 95 - DNA, 46 bp, Artificial Sequence
  Synthetic Sequence - Staple DNA
ggaacttcagcccaactaacattttgtccatcacgcatcaatcgtc

SEQ ID NO: 96 - DNA, 53 bp, Artificial Sequence
  Synthetic Sequence - Staple DNA
ggaacttcagcccaactaacatttttttgcccgaacgcgtattaagcaaatga

SEQ ID NO: 97 - DNA, 49 bp, Artificial Sequence
  Synthetic Sequence - Target Gene
gcgtgatcgatctcgatcccgcgaaattaatacgactcactatagggat

SEQ ID NO: 98 - DNA, 160 bp, Artificial Sequence
  Synthetic Sequence - Target gene
gctccatagctttaaatcgatgggatgatccggataacgggaaaagcattgaacaccataagttaaagtagtgacaagtgttggccatggaacagacagctcctcgcccttgctcaccatatccctatagtgagtcgtattaatttcgcggacgcctgca

SEQ ID NO: 99 - DNA, 21 bp, Artificial Sequence
  PCR Primer
ttgttggacgacccagacatc

SEQ ID NO: 100 - DNA, 21 bp, Artificial Sequence
  PCR Primer
atggggtatttgagggtcagg

SEQ ID NO: 101 - DNA, 18 bp, Artificial Sequence
  PCR Primer
ctgttccatggccaacac

SEQ ID NO: 102 - DNA, 16 bp, Artificial Sequence
  PCR Primer
tgatccggataacggg

SEQ ID NO: 103 - RNA, 21 nt, Homo sapiens
ugagguaguagguuguauagu

SEQ ID NO: 104 - RNA, 21 nt, Homo sapiens
cuagguaguagguuguauagu

SEQ ID NO: 105 - RNA, 21 nt, Homo sapiens
uucgguaguagguuguauagu

SEQ ID NO: 106 - RNA, 21 nt, Homo sapiens
ugucguaguagguuguauagu

SEQ ID NO: 107 - RNA, 21 nt, Homo sapiens
ugaucuaguagguuguauagu

SEQ ID NO: 108 - RNA, 21 nt, Homo sapiens
ugagucaguagguuguauagu

SEQ ID NO: 109 - RNA, 21 nt, Homo sapiens
ugagggcguagguuguauagu

SEQ ID NO: 110 - RNA, 21 nt, Homo sapiens
ugagguucuagguuguauagu

SEQ ID NO: 111 - RNA, 21 nt, Homo sapiens
ugagguaaaagguuguauagu

SEQ ID NO: 112 - RNA, 21 nt, Homo sapiens
ugagguagcugguuguauagu

SEQ ID NO: 113 - RNA, 21 nt, Homo sapiens
ugagguaguucguuguauagu

SEQ ID NO: 114 - RNA, 21 nt, Homo sapiens
ugagguaguaaauuguauagu

SEQ ID NO: 115 - RNA, 21 nt, Homo sapiens
ugagguaguagucuguauagu

SEQ ID NO: 116 - RNA, 21 nt, Homo sapiens
ugagguaguaggaaguauagu

SEQ ID NO: 117 - RNA, 21 nt, Homo sapiens
ugagguaguagguaauauagu

SEQ ID NO: 118 - RNA, 21 nt, Homo sapiens
ugagguaguagguuagauagu

SEQ ID NO: 119 - RNA, 21 nt, Homo sapiens
ugagguaguagguugaguagu

SEQ ID NO: 120 - RNA, 21 nt, Homo sapiens
ugagguaguagguugucgagu

SEQ ID NO: 121 - RNA, 21 nt, Homo sapiens
ugagguaguagguuguaacgu

SEQ ID NO: 122 - RNA, 21 nt, Homo sapiens
ugagguaguagguuguauucu

SEQ ID NO: 123 - RNA, 21 nt, Homo sapiens
ugagguaguagguuguauauc

SEQ ID NO: 124 - RNA, 22 nt, Homo sapiens
uggaauguaaagaaguauguau

SEQ ID NO: 125 - RNA, 22 nt, Homo sapiens
uggaauguaaggaagugugugg

SEQ ID NO: 126 - RNA, 22 nt, Homo sapiens
uauugcacuugucccggccugu

SEQ ID NO: 127 - RNA, 22 nt, Homo sapiens
uucaccaccuucuccacccagc

SEQ ID NO: 128 - RNA, 21 nt, Homo sapiens
caagucacuagugguuccguu

SEQ ID NO: 129 - RNA, 22 nt, Homo sapiens
uaaugccccuaaaaauccuuau

SEQ ID NO: 130 - RNA, 22 nt, Homo sapiens
uauggcacugguagaauucacu

SEQ ID NO: 131 - DNA, 21 bp, Artificial Sequence
  Synthetic Sequence - LacO1
aattgtgagcggataacaatt

SEQ ID NO: 132 - DNA, 20 bp, Artificial Sequence
  Synthetic Sequence - LacOid
aattgtgagcgctcacaatt

(19) United States
(12) Patent Application Publication
(10) Pub. No.: US 2020/0370136 A1
(43) Pub. Date: Nov. 26, 2020

(54) PROGRAMMABLE GENE EXPRESSION SYSTEM

(71) Applicant: Hisashi TADAKUMA, Tokyo (JP)

(72) Inventors: Hisashi TADAKUMA, Suita (JP); Takeya MASUBUCHI, Tokyo (JP); Yoshie HARADA, Suita (JP)

(73) Assignee: Hisashi TADAKUMA, Tokyo (JP)

(21) Appl. No.: 16/768,781

(22) PCT Filed: Nov. 30, 2018
(86) PCT No.: PCT/JP2018/044292

§ 371 Date: Jun. 1, 2020

(30) Foreign Application Priority Data
Dec. 1, 2017 (JP) 2017-232193
Jul. 18, 2018 (JP) 2018- 135358

(51) Int. Cl. C12Q 1/6897; C12N 15/67

(52) U.S. Cl. CPC C12Q 1/6897; C12Q 2565/629; C12N 15/67

(57) ABSTRACT

An object to the present invention is to provide a device for genetic circuits that is under extremely reduced restrictions on the materials of a sensor sensing the environment, capable of performing controlled gene expression with sufficiently suppressed crosstalk, and a genetic circuit containing the device. The present invention relates to a device for a genetic circuit, containing an open-close mechanism constituted of a catalyst, a target gene, and a shape change element, wherein the catalyst induces an expression of the target gene by contacting the target gene, and the like.

### TECHNICAL FIELD

The present invention relates to a programmable gene expression system, and specifically relates to a device for a gene circuit, and a gene circuit containing the device.

### BACKGROUND ART

In synthetic biology, the regulation of gene expression is a fundamental goal. The key steps in the regulation of gene expression are sensing the environment, computing information, and outputting the results. To achieve such function, it is necessary to laboriously construct a network of catalysts and substrate genes in combination.

In conventional gene expression systems in gene circuits, the relevant factors (catalysts and substrates) are based on a reaction diffusion system that drifts freely in a solution and must find each other by diffusion to cause a biochemical reaction. Thus, this system has two major drawbacks: (1) the biochemical reaction depends on the concentration of catalyst, substrate and other components, and (2) the nonspecific binding of catalyst and substrate causes crosstalk between gene circuits. Therefore, the development of a useful gene circuit requires laborious and time-consuming attempt to fine tune the concentration and activity of the constituent elements, limiting the construction of plural orthogonal gene reactions. Since the reaction rate depends on the frequency of collision of catalyst and substrate, low concentrations of the catalyst and substrate decrease the reaction rate, which imposes limitation on the multistep reaction. Moreover, conventional gene circuits have suffered from the material limitation of the sensor, which senses the environment and thus is the key for gene expression regulation. This is because the sensor need to be the substrate of a catalyst. To solve these problems, semiconductor design methods were used and automated design methods were introduced in non-patent document 1. However, the complexity was limited because non-specific interactions could not be eliminated.

### DOCUMENT LIST

### Non-Patent Document

non-patent document 1: Nielsen et al., Science. 352, aac7341, 2016

### SUMMARY OF INVENTION

### Technical Problem

The problem is to provide a new device for gene circuits that is under extremely reduced restrictions on the materials of a sensor sensing the environment and that can overcome two major drawbacks of the gene expression system in conventional gene circuits that (1) the biochemical reaction depends on the concentrations of the catalyst, substrate, and other components, and (2) the non-specific binding of the catalyst to the substrate causes crosstalk between gene circuits, and a gene circuit containing the device.

### Solution to Problem

To overcome the drawbacks of the above-mentioned (1) and (2), the present inventors took note of controlling the intermolecular distance between catalyst and substrate, and studied the controlling method. They have conducted intensive studies and found that the intermolecular distance between the catalyst and the substrate can be controlled by using a linker capable of expanding-contacting motion, and deploying-folding motion. As a result, the present inventors achieved control of the frequency of contact between the catalyst and the substrate, where generation of crosstalk is suppressed, which resulted in the completion of the present invention. In addition, since the present inventors found a method for controlling the intermolecular distance between the catalyst and the substrate by using a linker, a sensor that detects the environment does not need to the substrate of the catalyst, and they have also succeeded in markedly reducing the restriction on the materials. Furthermore, the present inventors also found a method for changing the logical operation by arbitrarily changing the intermolecular distance between the catalyst and the substrate, and also succeeded in adding a function of reprogramming.

That is, the present invention provides the following.
[1] A device for a gene circuit, comprising an open-close mechanism constituted of a catalyst, a target gene, and a shape change element, wherein

the aforementioned catalyst induces an expression of the aforementioned target gene by contacting the aforementioned target gene,

the aforementioned shape change element has a first site and a second site, and has a structure in which a distance between the aforementioned first site and the second site changes by an action of an activation source, and

the aforementioned open-close mechanism has the aforementioned shape change element as a movable part, and is configured to perform an open-close motion in which the aforementioned target gene and the aforementioned catalyst relatively approach and contact with each other or relatively leave the contact state in response to a change in the distance between the first site and the second site in the aforementioned shape change element.
[2] The device according to [1], further comprising a base member,

either one of the aforementioned catalyst and target gene is fixed on (one plane of) the aforementioned base member, and the other is fixed on the side of the first site of the shape change element in the movable part,

the side of the second site of the shape change element in the movable part is fixed on (the aforementioned plane of) the aforementioned base member,

the aforementioned open-close mechanism is configured such that the catalyst and the target gene relatively approach and contact with each other or relatively leave the contact state by an increase in the distance between the first site and the second site in the aforementioned shape change element, and

the aforementioned open-close mechanism is configured such that the aforementioned catalyst and the target gene relatively approach and contact with each other or relatively leave the contact state by a decrease in the distance between the first site and the second site in the aforementioned shape change element.
[3] The device according to [1], further comprising a base member, a first shape change element as a mutually independent first movable part, and a second shape change element as a mutually independent second movable part,

the aforementioned catalyst is fixed on the side of a first site of the first shape change element in the first movable part,

the target gene is fixed on a first site side of the second shape change element in the second movable part,

the second site side of each shape change element in the first and the second movable parts is fixed (separately from each other on one plane of) the aforementioned base member,

the aforementioned open-close mechanism is configured such that the aforementioned catalyst and the target gene relatively approach and contact with each other or relatively leave the contact state by an increase in the distance between the first site and the second site in each shape change element, and

the aforementioned open-close mechanism is configured such that the catalyst and the target gene relatively approach and contact with each other or relatively leave the contact state by a decrease in the distance between the first site and the second site in each shape change element.
[4] The device according to [1], further comprising a (sheet-like) base member having flexibility, wherein

the aforementioned catalyst and the target gene are placed (separately from each other) on (one plane, one of the planes, or a certain plane) of the aforementioned (sheet-like) base member, the first site and the second site of the aforementioned shape change element are fixed (separately from each other on the same plane) of (the aforementioned sheet-like) base member, and the aforementioned open-close mechanism is configured such that a decrease or an increase in the distance between the first site and the second site deforms (the aforementioned sheet-like) base member, and the catalyst and the target gene relatively approach and contact with each other or relatively leave the contact state by the deformation of (the aforementioned sheet-like) base member.
[5] The device described in any of [1] to [3], wherein the aforementioned movable part has shape change elements in the number of n which are connected to each other, wherein n is an integer of 2 or more, and the movable part is configured such that the aforementioned target gene and the aforementioned catalyst relatively approach and contact with each other or relatively leave the contact state when the distance between the first site and the second site changes in at least one of the shape change elements among the aforementioned shape change elements in the number of n.
[6] The device according to [5], wherein the device is configured such that the aforementioned target gene and the aforementioned catalyst relatively approach and contact with each other or relatively leave the contact state when the distance between the first site and the second site in all of the aforementioned shape change elements in the number of n changes.
[7] The device described in any of [1] to [3], wherein the aforementioned movable part has shape change elements in the number of n which are connected to each other, wherein n is an integer of 3 or more, and the movable part is configured such that the aforementioned target gene and the aforementioned catalyst relatively approach and contact with each other or relatively leave the contact state when the distance between the first site and the second site changes in not less than half of the shape change elements among the aforementioned shape change elements in the number of n.
[8] The device according to [2], wherein either one of the aforementioned catalyst and target gene is fixed on a first anchor part on (one plane of) the aforementioned base member, and the other is fixed on a first fixed part on the side of the first site of the shape change element in the movable part,

a second fixed part on the side of the second site of the shape change element in the movable part is fixed on a second anchor part on (the aforementioned plane) of the aforementioned base member,

a third fixed part is provided between the first fixed part and the second fixed part of the movable part, a third anchor part is provided on (the aforementioned plane) of the base member at a position where the third fixed part of the movable part can reach, and

the third anchor part and the third fixed part are configured to be bondable to each other.
[9] The device according to [8], wherein the aforementioned third anchor part is located between the aforementioned first anchor part and the second anchor part.
[10] The device according to [8] or [9], wherein the aforementioned third anchor part and the third fixed part are bondable to each other by an action of an activation source for bonding.
[11] The device according to [8] or [9], wherein the aforementioned third anchor part and the aforementioned third fixed part are separable from each other by an action of an activation source for release when the aforementioned third fixed part is bonded to the aforementioned third anchor part.
[12] The device described in any of [8] to [11], wherein the aforementioned movable part has a plurality of shape change elements which are connected (in tandem), and the aforementioned third fixed part is located between the plurality of the shape change elements.
[13] The device described in the aforementioned [2], wherein either one of the aforementioned catalyst and target gene is m fixed on (one plane of) a first anchor part of the aforementioned base member, and the other is fixed on a first fixed part on the side of the first site of the shape change element in the movable part,

a second fixed part on the side of the second site of the shape change element in the movable part is fixed on a second anchor part on (the aforementioned plane) of the aforementioned base member,

a third anchor part is provided on (the aforementioned plane) of the base member, a third fixed part is provided between the first fixed part and the second fixed part of the movable part,

the third anchor part and the third fixed part are bonded to each other such that they are separable from each other by an action of an activation source for release,

the position of the third anchor part on (on the aforementioned plane) of the base member is selected such that the first fixed part does not reach the first anchor part, and the first fixed part is configured to reach the first anchor part when the third anchor part and the third fixed part are separated from each other by an action of the activation source for release, whereby the aforementioned target gene and the aforementioned catalyst relatively approach and contact with each other.
[14] The device described in the aforementioned [2], wherein either one of the aforementioned catalyst and target gene is fixed on (one plane of) a first anchor part of the aforementioned base member, and the other is fixed on a first fixed part on the side of the first site of the shape change element in the movable part,

a second fixed part on the side of the second site of the shape change element in the movable part is fixed on a second anchor part on (the aforementioned plane) of the aforementioned base member,

a third anchor part is provided on (the aforementioned plane) of the base member, a third fixed part is provided between the first fixed part and the second fixed part of the movable part,

the third anchor part and the third fixed part are bonded to each other such that they are optionally separated from each other by an action of an activation source for release,

the position of the third anchor part on (on the aforementioned plane) of the base member is selected such that the distance between the first anchor part and the third anchor part is equal to or longer than the distance between the first fixed part and the third fixed part, whereby the aforementioned target gene and the aforementioned catalyst come into contact.
[15] The device described in the aforementioned [14], wherein the distance between the first fixed part and the second fixed part is shorter or longer than the distance between the first anchor part and the second anchor part by a change in the distance between the first site and the second site of the shape change element when the third anchor part and the third fixed part are separated from each other by an action of the activation source for release.
[16] The device described in any of [2] to [4] and [8] to [15], wherein the aforementioned base member is a nanostructure composed of DNA, RNA, artificial nucleic acid, peptide, protein, or polymer, or a combination thereof.
[17] The device described in any of [1] to [16], wherein the aforementioned catalyst is RNA polymerase, DNA polymerase, artificial nucleic acid polymerase, or polymer synthase, the aforementioned shape change element is nucleic acid, artificial nucleic acid, or polymer, and the aforementioned activation source is miRNA, RNA, DNA, artificial nucleic acid, polymer LacI protein, IPTG (Isopropyl β-D-1-thiogalactopyranoside) compound, Ampicillin antibiotic, or light.
[18] A gene circuit comprising one or more devices described in any of [1] to [17], and having a logic circuit configured to perform a logical operation by the open-close mechanism of the aforementioned one or more devices.
[19] The gene circuit according to [18], having one or both of a logic circuit of the following (I) and a logic circuit of the following (II):

(I) a logic circuit configured by the above-mentioned one or more devices and having one or more switches selected from ON switch, OFF switch, ON/OFF selector switch, AND switch, OR switch, NAND switch, NOR switch, and MAJORITY switch,

(II) a logic circuit configured by the above-mentioned one or more devices and having two or more switches selected from ON switch, OFF switch, ON/OFF selector switch, AND switch, OR switch, NAND switch, NOR switch, and MAJORITY switch.

### Advantageous Effects of Invention

The device of the present invention is characterized in that it has an open-close mechanism configured to control the positional or contact relationship between the catalyst and the target gene to each other. The open-close mechanism is

(i) configured to perform an action of bringing the catalyst and the target gene in a distant (open) state into close contact with each other (i.e., a closing action to close an open circuit); or

(ii) configured to perform an action to separate the catalyst in contact with each other (closed state) from the target gene (i.e., an open action to open a closed circuit).

The action of bringing the catalyst and the target gene into contact with each other includes not only the action of completing the contact between the two, but also the action of increasing the probability of the contact between the two. In addition, the action of separating the catalyst and the target gene in a state of contact with each other includes the action of lowering the probability of contact between the two on the catalyst and the target gene under high probability of contact with each other.

The present invention uses one or more shape change elements as a moving part (driving source) for achieving the closing motion and opening motion (open-close motion) of the open-close mechanisms of the aforementioned (i), (ii).

The shape change element has a first site and a second site, and has a structure in which a distance between the first site and the second site changes by an action of an activation source (e.g., applied from the outside), as in a typical example, a stem-loop structure. The change in the distance between the first site and the second site may be an increase or decrease in distance due to the expanding-contacting motion of the shape change element, or an increase or decrease in distance due to the deploying-folding motion of the shape change element. For example, in the case of a stem-loop structure, when only the two end portions thereof (i.e., the base end portion of the two straight-line segments of the stem) are focused on, it may be understood that the distance between the two end portions simply increases or decreases, or when the form of the entire stem-loop structure is focused on, it may be understood that the distance between the two end portions of the stem-loop structure increases or decreases by the deploying or folding motion of the entire stem-loop structure.

A plurality of shape change elements may be linked in a row to form a single movable part. The device optionally has two or more movable parts. When the device includes a plurality of shape change elements, whether those plurality of shape change elements are considered to be one movable part or any plurality of movable parts can be appropriately selected in accordance with the motion of each shape change element, the intended purpose, the grouping for convenience, and the like.

The shape change element may be an element permitting change of the whole shape, like a stem loop structure in RNA, or an element including an arm part connected at one end or both ends thereof. In the following, elements permitting change of the whole shape, such as the above-mentioned stem-and-loop structure, are called the sensor part and distinguished from the arm part that does not contribute to the change in shape. Accordingly, the configuration of the shape change element includes a configuration of the sensor part alone, a configuration in which an arm part is connected to one end of the sensor part, a configuration in which an arm part is connected to each of the both ends of the sensor part, and the like.

The first site and the second site of the shape change element may be, in a typical example, the sites of the both ends of the sensor part. They may be any two sites in the shape change element including the arm part. They only need to be sites selected such that the distance between the two sites changes by an action of an activation source (e.g., applied from the outside).

The open-close mechanism may be constructed as a mechanism having various configurations for open and close a catalyst and a target gene by utilizing changes in the distance between the first site and the second site of a shape change element that is a movable part.

For example, the first site and the second site of the shape change element not only permit an increase or decrease in the distance between each other, but also be in contact with and away from each other, such as the two base end portions of the stem part in the stem-loop structure. Therefore, in such shape change element permitting contact with and separation from each other, an open-close motion between the catalyst and the target gene can be achieved by attaching a catalyst to the first site of the shape change element and a target gene to the second part. Even when the first site and the second site of the shape change element cannot contact with each other directly and are in a relationship where the distance between them merely increases or decreases, the increase or decrease of the distance can be used to construct a configuration that opens and closes the catalyst and the target gene, for example, like a bimetallic structure.

When the first site and the second site of the shape change element cannot contact with each other directly and are in a relationship where the distance between them merely increases or decreases, the distance between the second site and a reference position increases or decreases when the first site is fixed at the reference position on the plane of a certain base member. Thus, when a catalyst (or target gene) is attached to the second site and a target gene (or catalyst) is attached to another site present at a predetermined distance from the aforementioned reference position, the catalyst and the target gene can be contacted or separated according to the increase or decrease in the distance between the first site and the second site of the shape change element (including the possible increase or decrease in the probability of contact between the catalyst and the target gene). The position of the above-mentioned reference position of the base member and the position of another site present at a predetermined distance therefrom may be two points on the same base member or may be one point each on two members in a predetermined positional relationship with each other.

In a preferred embodiment of the present invention, the device has a base member. In the present embodiment, the catalyst (which may be a target gene) is fixed on one plane of the base member, and the target gene (which may be a catalyst) is fixed on the side of the first site of the shape change element in the movable part. In addition, the side of the second site of the shape change element in the movable part is fixed on the aforementioned plane of the base member. In this way, for example, when the catalyst and the target gene are separated from each other due to the shortness of the whole movable part, the catalyst and the target gene can be brought closely into contact by increasing the distance between the first site and the second site in the aforementioned shape change element. When the catalyst and the target gene are in contact with each other, the contact between the catalyst and the target gene can be cancelled by an increase in the distance between the first site and the second site in the shape change element (hereinafter to be also referred to as the length of the shape change element). Conversely, when the catalyst and the target gene are separated from each other because the entire moving part is too long, the catalyst and the target gene can be brought closely into contact by decreasing the length of the shape change element. When the catalyst and the target gene are in contact with each other, the catalyst and the target gene can be separated by a decrease in the length of the shape change element.

In a preferred embodiment of the present invention, the device has a base member, and two movable parts (a first movable part and a second movable part) that are independent of each other. In the present embodiment, the catalyst is fixed on the side of the first site of the first shape change element in the first movable part, and the target gene is fixed on the side of the first site of the second shape change element in the second movable part. On the other hand, the sides of the second site of each shape change element are fixed separately from each other on one plane of the base member. In this case, for example, when the catalyst and the target gene are separated from each other due to the shortness of the whole movable part of the both, the catalyst and the target gene can be brought closely into contact by increasing the length of the shape change element. When the catalyst and the target gene are in contact with each other, the contact between the catalyst and the target gene can be cancelled by an increase in the length of the shape change element. Conversely, when the catalyst and the target gene are separated from each other because the entire moving part of the both is too long, the catalyst and the target gene can be brought closely into contact by decreasing the length of the shape change element. When the catalyst and the target gene are in contact with each other, the catalyst and the target gene can be separated by a decrease in the length of the shape change element.

In a preferred embodiment of the present invention, the device has a base member with flexibility. A catalyst and a target gene are disposed separated from each other on one plane (first plane) of the base member. The first site and the second site of the shape change element are fixed apart from each other on the same plane of one plane or another plane (the second plane) of the base member. In this way, for example, when the catalyst, the target gene, and the shape change element are disposed on the same plane (e.g., the first plane) of the base member, a decrease in the length of the shape change element may cause the base member to be curved to bring the catalyst and the target gene into contact on the same plane. When the length of the shape change element increases, the curvature is released or bent in reverse to pull the catalyst and the target gene apart. On the other hand, when the catalyst and the target gene are disposed on the first plane of the base member and the shape change element is placed on the second plane on the backside, a decrease in the length of the shape change element changes the base member and the catalyst and the target gene on the side of the first plane can be separated. When the length of the shape change element increases, the sheet-like base member changes opposite and can bring the catalyst and the target gene into contact.

In a more specific embodiment included in the above-mentioned preferred embodiment of the present invention, for example, the base member having flexibility is a sheet-like base member. A catalyst and a target gene are disposed apart from each other on one plane (first plane) of the sheet-like base member. The first site and the second site of the shape change element are fixed apart from each other on the same plane of the first plane or another plane (the second plane) of the sheet-like base member. In this way, for example, when the catalyst, the target gene, and the shape change element are disposed on the same plane (e.g., the first plane) of the base member, a decrease in the length of the shape change element may cause the sheet-like base member to be curved to bring the catalyst and the target gene into contact on the same plane. When the length of the shape change element increases, the curvature is released or bent in reverse to pull the catalyst and the target gene apart. On the other hand, when the catalyst and the target gene are disposed on the first plane of the base member and the shape change element is placed on the second plane on the backside, a decrease in the length of the shape change element curves the sheet-like base member and the catalyst and the target gene on the side of the first plane can be separated. When the length of the shape change element increases, the sheet-like base member curves opposite and can bring the catalyst and the target gene into contact.

In a preferred embodiment of the present invention, the device has a base member, the catalyst (which may be a target gene) is fixed on a first anchor part on one plane of the base member, the target gene (which may be a catalyst) is fixed on the first fixed part on the side of the first site of the shape change element in the movable part, and the second fixed part on the side of the second site of the shape change element in the movable part is fixed on the second anchor part on the aforementioned plane of the base member. Furthermore, a third fixed part is provided between the first fixed part and the second fixed part of the movable part, and a third anchor part is provided on the aforementioned plane of the base member at a position where the third fixed part of the movable part can be reached. The third anchor part of the base member and the third fixed part of the movable part can be bonded to each other. In this way, when the third anchor part is not bonded to the third fixed part, the fixed end portion of the movement of the whole movable part is the second fixed part. When the third anchor part is bonded to the third fixed part, the fixed end portion of the movement of the whole movable part moves to the third fixed part. This movement of the fixed end portion can increase the probability of contact between the catalyst and the target gene (e.g., by shortening the length of the freely movable part of the whole movable part and limiting the range of motion of the movable part).

When the movable part has multiple shape change elements, it is also possible to change the shape change element to be actuated to open and close the catalyst and the target gene to another shape change element by placing a third fixed part between the shape change elements. A change in the shape change element by the output of open-close of the catalyst and the target gene means a change in the input conditions, which means a change in the logic of the input-output in a single circuit.

In a preferred embodiment of the present invention, the device has a base member, the catalyst (which may be a target gene) is fixed on a first anchor part on one plane of the base member, the target gene (which may be a catalyst) is fixed on the first fixed part on the side of the first site of the shape change element in the movable part, and the second fixed part on the side of the second site of the shape change element in the movable part is fixed on the second anchor part on the aforementioned plane of the base member. Furthermore, a third anchor part is provided on the aforementioned plane of the base member, and a third fixed part is provided between the first fixed part and the second fixed part of the movable part. The third anchor part and the third fixed part are releasably bonded to each other by the action of an activation source for release, and the position of the third anchor part on the aforementioned plane of the base member is selected such that the first fixed part does not reach the first anchor part. In this way, the first fixed part can reach the first anchor part when the third anchor part and the third fixed part are separated from each other due to the action of the activating source for release, and the target gene and the catalyst can relatively approach and contact with each other. This operation can be said to turn a switch that was initially OFF into ON by the input of an action of an activation source for release.

In a preferred embodiment of the present invention, the device has a base member, the catalyst (which may be a target gene) is fixed on a first anchor part on one plane of the base member, the target gene (which may be a catalyst) is fixed on the first fixed part on the side of the first site of the shape change element in the movable part, and the second fixed part on the side of the second site of the shape change element in the movable part is fixed on the second anchor part on the aforementioned plane of the base member. Furthermore, a third anchor part is provided on the aforementioned plane of the base member, and a third fixed part is provided between the first fixed part and the second fixed part of the movable part. The third anchor part and the third fixed part are releasably bonded to each other by the action of an activation source for release, and the position of the third anchor part on the aforementioned plane of the base member is selected such that the distance between the first anchor part and the third anchor part is equal to or longer than the distance between the first fixed part and the third fixed part. In this way, when the third anchor part and the third fixed part are connected to each other, the catalyst and the target gene can come into contact with each other with a high probability or can actually come into contact with each other.

In this embodiment, when the third anchor part and the third fixed part are released from each other by the action of the activation source for release, the length of the freely movable part in the movable part is increased, the limitation on the motion range of the movable part is released, and the probability of contact between the catalyst and the target gene is lowered more. In addition, when the third anchor part and the third fixed part are separated from each other and the length of the shape change element is changed, the distance between the first fixed part and the second fixed part is changed, the distance between the first fixed part and the second fixed part can be made shorter or longer than the distance between the first anchor part and the second anchor part. In this way, the catalyst and the target gene can be relatively separated from the contact state.

The device of the present invention functions as various switches and logic switches such as ON switch, OFF switch, ON/OFF selector switch, AND switch, OR switch, NAND switch, NOR switch, MAJORITY switch and the like, as described below. Therefore, by using one or more devices and/or combining two or more thereof, a gene circuit having a logic circuit configured to perform various logical operations can be provided. Examples of the gene circuit include a circuit containing one or more switches selected from the aforementioned ON switch, OFF switch, ON/OFF selector switch, AND switch, OR switch, NAND switch, NOR switch, and MAJORITY switch, a circuit that optionally combines two or more of these switches to perform various logical operations (the above-mentioned two or more switches may be those in which the same switch is selected in duplicate). The gene circuit may optionally include the aforementioned switches and circuits according to the application.

According to the present invention, a new device for gene circuits that is under extremely reduced restrictions on the materials of a sensor sensing the environment, and a gene circuit containing the device are provided. They are expected to be usable as a gene circuit capable of performing controlled gene expression with sufficiently suppressed crosstalk for the treatment of gene diseases, biomarker detection, and efficient synthesis of biopharmaceuticals.

### BRIEF DESCRIPTION OF THE DRAWINGS

FIG. 1 shows assembly and activity of the T7-chip. (FIG. 1A) SNAP-ligand handle was introduced at a specific position in a rectangle-type DNA tile origami, and a SNAPf protein fused T7 RNAP (T7 RNAP_{SNAPf}) was then added. The complex of the DNA origami and T7 RNAP_{SNAPf} (T7-chip) was then purified with magnetic beads using the toehold elution method. (FIG. 1B) Samples were resolved by 1% agarose gel electrophoresis, which showed that the unbound free T7 RNAP_{SNAPf} (arrowhead in the left gel, Cy5 channel) was removed by the purification process. The arrow for the right gel (Cy3 channel) indicates the position of the rectangle-type tile (Rect-tile). (FIG. 1C) Results of 12% poly-acrylamide gel electrophoresis (SDS.PAGE) also confirmed that the unbound free T7 RNAP_{SNAPf} (arrowhead) has been removed. The arrow indicates the position of the bound T7 RNAP_{SNAPf}. ctrl, control lane showing the position of free T7 RNAP_{SNAPf}. (FIG. 1D) AFM image of the T7-chip. The T7 RNAP_{SNAPf} was attached near the left bottom corner. Yield: 95% (222/234 tiles). Scale bar, 20 nm. (FIG. 1E) Schematic illustration of the experiment evaluating the kinetic character of the T7-chip. (FIG. 1F) The transcription activity of the T7-chip (below) follows Michaelis-Menten kinetics. The apparent Km value (1,010 nM) is higher than that of the freely diffusing T7 RNAPSNAPf. Additionally, the estimated Vmax of the T7-chip is lower than that of the freely diffusing T7 RNAPSNAPf, with values of 34±4 and 62±0.9 nM s−1, respectively. Taken together, these data show that the T7-chip has low affinity for externally diffusing genes.

FIG. 2 shows properties and rational design of the gene nanochip activity. (FIG. 2A) Molecular layout of five gene nanochips. Black circles and black squares indicate positions of the T7 RNAP_{SNAPf} and target gene, respectively. Scale bar, 50 nm. (FIG. 2B) Transcription activity of genes attached at the 5′ (gray circle) and 3′ (black circle) ends, where the 5′ end close to the promoter sequence is defined as the 5′ end. Error bars indicate s.d. of three independent experiments. The transcription activity was converted to a value corresponding to that of 10 nM T7 RNAP. (FIG. 2C) Left, schematic illustration of the competition assay. Top right, results for 4% urea-PAGE analysis. Endo, transcripts of the sfGFP gene anchored on a nanochip (942 nt); Exo, transcripts of the freely diffusing competitor DHFR gene (682 nt). The assay was performed in transcription buffer, and the concentration of the nanochip was ˜1 nM. Bottom right, quantification of the urea-PAGE data, indicating that the effective concentration of the endogenous gene on the DNA origami is greater than 2 uM. (FIG. 2D) Top, schematic illustration of the T7-gene-chip. P, promoter sequence; Linker, double-stranded linker DNA between the anchor points and the promoter sequence. Bottom, optimal T7 RNAP_{SNAPf}-gene distance is dependent on linker length. The assay was performed in transcription buffer. The transcription activity was converted to a value corresponding to that of 10 nM T7 RNAP. (FIG. 2E) Expression ratio of two genes can be controlled by changing the molecular layout of the respective genes. In this set of experiments, the position of T7 RNAP_{SNAPf} (black circle) and the position of the mCherry gene (dark gray rectangle) were fixed, but the position of the sfGFP gene (gray rectangle) was varied. Thus, in contrast to the conventional approach of ratio control, where the molar ratio of the target gene or promoter sequence is changed (top right inset), the nanochip uses the same materials but changes the molecular layout. The assay was performed in a PURE system, and the concentration of the nanochip was ˜1 nM. The reaction times were 300, 600, 1,800 and 3,600 s (5, 10, 30 and 60 min), and the fluorescence image was taken at 3,600 s.

FIG. 3 shows that a single nanochip can express a defined gene in an artificial cell. (FIG. 3A) Left, microfluidic device used in the experiments. The w/o droplets, which encapsulate the PURE system, were made using an air presser. Scale bar, 20 um. Right, fluorescence intensity of sfGFP expressed in each cell. Nanochip activity (black circles) was compared with the activity of the DNA- (gray circles) and mRNA-initiated (black squares) reactions. Bottom right, representative images of the cell at 0.8 pM nanochip concentration, showing the heterogeneous intensity of sfGFP fluorescence (left half). The right half shows the phase contrast image. (FIG. 3B) Left, histogram of cells at the indicated target-gene concentrations. The arrow indicates the intensity of the background signal. Single and double arrowheads indicate distinctive peaks corresponding to cells containing one or two genes. The inventors note that the concentration of T7 RNAP_{SNAPf} is 100 nM for the DNA-initiated reaction (bottom blue bin graph).

FIG. 4-1 shows that nano-chip based genetic circuit autonomously responds to the miRNA profile of an artificial cell. (FIG. 4A) Schematic drawing explaining the structure of the ON switch. Following small RNA (miRNA) binding, an extension of the effective arm length of the target-gene allowed the interaction of RNAP with the promoter sequence. Inset, Addition of let-7 miRNA activates the let-7 ON switch. w/, with miRNA; w/o, without miRNA. (FIG. 4B) Orthogonality of the ON switch. The activity of each sensor was normalized by the sfGFP output measured for the switch with its cognate trigger miRNA. (FIG. 4C) The three-input AND switch responds to let-7, miR-206 and miR-92a-3p. Note that the single three-input AND switch activity was confirmed by w/o droplet experiments (see also FIG. 23A-E). (FIG. 4D) Inset, with specific linker length, the activity of the nanochip showed a peak along the intermolecular distance (see also FIG. 2D). Conversely, with the specific intermolecular distance, there should be an optimal linker length. Therefore, if the effective linker length was changed following small RNA (miRNA) binding, from the non-optimal length (for example, too short) to the optimal one, the chip was activated (FIG. 4A). In addition, if the inventors assumed the tipping point of the effective linker length for the OFF-to-ON transition, the inventors could design the chip such that a specific number of bound miRNAs induces sufficient extension of the effective linker length to cross the tipping point (for example, one miRNA binds for three-input OR, two miRNAs bind for three-input Majority and three miRNAs bind for three-input AND switch). Outer schematics, in the three-input AND switch, for example, three bound miRNAs are required to cross the tipping point (upper left; see also FIG. 24A-D). If the inventors reduce the intermolecular distance between the enzyme and the target-gene anchoring points that is equivalent to the extension achieved by two bound miRNA, the integrated sensor might now act as a three-input OR switch that is activated by binding of one miRNA. (FIG. 4E) Function of the three-input AND switch can be changed to a three-input OR switch (upper) and a three-input Majority switch (lower) by changing the nanochip configuration. The left side of the FIG. 4E shows the molecular layout of the nanochip and its equivalent switch. Error bars indicate s.d. of three independent experiments. (FIG. 4F) Schematic drawing to explain the crosslinking and reverse reaction of the photoreactive switch using the photoreactive crosslinking nucleotide (cnvK).

FIG. 4-2 (FIG. G) shows reprogramming of three-input AND switch to Majority switch by UV irradiation. FIG. 4-2 (FIG. H) shows genetic circuit consisting of two AND chips autonomously senses the miRNA profile of a w/o droplet, executes the logic operation, and responds. In the circuit, Chip 1 detects the miR-206 and miR-92a-3p and produces the communication transmitter miRNA (let-7). Chip 2 produces the sfGFP mRNA only in the presence of both miR-197-3p and transmitter miRNA (let-7). Box plots showing the distribution of the fluorescence intensity of sfGFP expressed in each cell. Scale bar, 20 um.

FIG. 5 shows the representative raw data of exogenous gene transcription with the T7-chip. (FIG. 5A) Image of 4% urea-PAGE gel. The reaction times were 300, 600 and 1800 s for 100, 250, 400 nM exogenous genes. And 300 and 600 s for 1,000 and 2,000 nM exogenous genes. After electrophoresis, [32P]-UTP was imaged by BAS-5000. (FIG. 5B) Quantification of the data in FIG. 5A. Data collected at 300 and 600 s were used for fitting. (FIG. 5C) The slopes of FIG. 5B were plotted against the exogenous gene concentration. Curve fitting was performed by assuming a Michaelis-Menten type reaction. The inventors note that the activity was corrected for that of 10 nM T7 RNAP.

FIG. 6 shows that dual-biotin ensures single gene assembly. (FIG. 6A) Schematic illustration of endogenous gene assembly. Dual-biotin was used both for the attachment of streptavidin (SA) to the DNA origami tile and the attachment of the endogenous gene to SA. (FIG. 6B) Typical agarose gel image of the endogenous gene assembly using dual-biotin (left) and normal biotin (right). The number of arrowheads indicates the number of attached genes per DNA origami tile.

FIG. 7 shows AFM and gel images and yields of the T7-gene-chip. “W/ gene” indicates observed ratio of the nano-chip containing the gene (=“T7-gene-chip+gene-chip”), which can be directly compared to the data from the gel analysis. n.d., not determined. The inventors first calculated, with the data of 32-, 50- and 70-nm, that the formation efficiency of the ternary complex (T7-gene-chip) to be 89+/−2% of the data obtained by gel analysis (containing both T7-gene-chip and gene-chip). Therefore, the inventors estimated the ternary complex formation efficiency of 4- and 24-nm distance chips to be 69%.

FIG. 8 shows typical raw data for the 5′ and 3′ attached T7-gene-chips. (FIG. 8A) Schematic illustration of the molecular layout of the 5′ and 3′ end fixed T7-chips (the intermolecular distance between the attachment positions for T7 RNAP and the gene is 50 nm). (FIG. 8B) Image of 4% Urea-PAGE gel. The reaction times were 300, 600, 1,800 and 3,600 s (5, 10, 30 and 60 min). (FIG. 8C) Quantification of the data in FIG. 8B. The concentrations of the T7-chip were 0.37 and 0.5 nM for the 5′ and 3′ fixed ends, respectively.

FIG. 9 shows the T7-gene-chip remained intact after the transcription reaction. (FIG. 9A) Typical image of 1% agarose gel. The reaction times were 0, 5, 10 and 60 min. The positions of the main band (T7-gene-chip) and an extra band (T7-chip: nano-chip without gene) are indicated by a line. (FIG. 9B) Quantification of the data in FIG. 9A.

FIG. 10 shows the typical raw data from the competition experiment. (FIG. 10A) Image of 4% urea-PAGE gel. The reaction times were 300, 600 and 1,800 s (5, 10 and 30 min). endo, endogenous gene transcript (sfGFP, 942 nt); exo, exogenous gene transcript (DHFR, 682 nt). (FIG. 10B) Quantification of the data in FIG. 10A.

FIG. 11 shows the orthogonality of the T7-chip. (FIG. 11A) Image of 4% urea-PAGE gel. The reaction times were 600, 1,800 and 3,600 s (10, 30 and 60 min). (FIG. 11B) Quantification of the data in FIG. 11A. The data from the 600- and 1,800-s reactions were used for fitting. (FIG. 11C) Relative activity. The values were corrected for those of 10 nM T7 RNAP concentration.

FIG. 12 shows the prediction of the enzyme-substrate collision efficiency curve. (FIG. 12A) For simplicity, the inventors considered a one-dimensional distribution. The inventors assumed the size dr of the RNAP's promoter recognition domain to be 0.1 nm. (FIG. 12B and C) Predicted collision efficiency assuming that the persistence length (lp) is 50 nm (b) and 30 nm (c). The plotted data were the same as in FIG. 2D, where 22 (blue circle), 44 (gray circle) and 66 (white circle) nm linker data were plotted. Assuming a worm-like chain model of DNA, the intermolecular dependency of the nano-chip approximately obeyed the predicted collision efficiency curve, although a smaller persistence length is required for shorter linker conditions (25-35 nm (right), smaller than the known value of 50 nm (left)). Three possible mechanisms could explain the discrepancy from the calculation: one reflecting a softer state of the DNA (gene) (references: R. S. Mathew-Fenn, R. Das, P. A. Harbury. Science 322, 446-449 (2008), and R. Vafabakhsh, T. Ha. Science 337, 1097-1101 (2012)), the second indicating a widening of the accessible range through the scanning ability of RNAP. The inventors speculated that according to the results of the sensor-integrated chip, the latter mechanism might explain this discrepancy.

FIG. 13 shows AFM images and yields of the dual-gene version of the T7-gene-chip. W/ gene, observed ratio of the nano-chip containing the gene, which can be directly compared to the data from the gel analysis.

FIG. 14 shows sfGFP intensity profile of droplets. (FIG. 14A-C) Intensity profile of (A), the nano-chip, (B), the DNA-initiated and (C), the mRNA-initiated reactions. The inventors note that the concentration of (C), the mRNA-initiated reaction, is much higher than that of (A), nano-chip, and (B), the DNA-initiated, reaction. (FIG. 14D) T7 RNAP concentration dependence of the fluorescence intensity produced by the expression of a single gene in a droplet. The gene expression level of the single nano-chip, which was approximately 1,500 (a.u.), is equivalent to that of 100 nM freely diffusing T7 RNAP_{SNAPf}.

FIG. 15 shows unit element capability of nano-chip. (FIG. 15A) At lower concentrations, the stochastic distribution of the components resulted in “inactive” cells that expressed only one of the genes out of the two-gene set. (FIG. 15B) Typical fluorescence intensity profile of dual-gene expression by the nano-chip. (FIG. 15C) Corresponding representative pie charts for the nano-chip and the DNA-initiated reactions. (FIG. 15D) Success ratio of the expression of the defined gene set. The co-expression ratio was defined as the average of (double positive cells/sfGFP-positive cells) and (double positive cells/mCherry-positive cells). The line indicates the expected value of the DNA-initiated reactions assuming a stochastic distribution.

FIG. 16-1 shows the features of sensor-integrated nano-chip. (FIG. 16A) Modulation of the inter-molecular distance between the enzyme and substrate enables switching of the nano-chip activity. Schematic illustration of the sensor used in this study. The linker between the anchoring points and the promoter sequence served as the sensor. As the linker part is independent from the enzymatic reaction, the sensor has no material limitations. (FIG. 16B) Sequence limitations on the operator and material limitations on the sensor for previous methods (reaction-diffusion system, left) and the proposed method (integrated chip, right). (FIG. 16C) Summary of the limitation. With the method of the present invention, the sensor has no sequence and material constraint.

FIG. 16-2 shows features of sensor-integrated nano-chip. (FIG. 16D) Environment dependency of the kinetics and response speed at low enzymes-substrates concentration for previous methods (left) and the proposed method (right). With the proposed method, the environment dependency is low. In addition, as all the required components are integrated on the same chip and thus have a high effective concentration, the respond rate is not dependent on the chip (enzyme-substrate) concentration. (FIG. 16E) Functional modulation of logic function by previous methods (left, using recombinase) and the proposed method (right). With the proposed method, different nano-chip configurations enabled different logic function, as the collision efficiency change upon signal binding can be designed through numerous approaches (e.g., linker length change, molecular layout change and scaffold transformation).

FIG. 17 shows crucial factors for sensor design and estimation of effective linker length. (FIG. 17A) Three crucial factors should be considered for sensor design: I. Rigidity of the sensor, II. scanning ability of RNAP and III. pulling force of RNAP, which extends the linker part of the sensor attached substrate-gene. (FIG. 17B) Schematic drawing to explain the structure of sensor-integrated nano-chip. The linker part comprising ssDNA- and dsDNA domains functioned as the sensor. The former ssDNA domain provides the miRNA-binding site, and the latter dsDNA domain serves as the rigid spacer. To design the sensor, evaluating the effective sensor arm length is important; therefore, predicting the effective length of the ssDNA and dsDNA domain is crucial. Thus, the inventors measured the chip activity with a fixed inter-molecular distance between enzyme and substrate (50 nm) while changing the length of linker comprising ssDNA (0-60 nt) and dsDNA (25-85 bp). (FIG. 17C) With an identical dsDNA linker length, the reach of the sensor arm with a short ssDNA linker is too short to activate the chip activity, but with a long ssDNA linker is of sufficient length. The inventors assumed the existence of a tipping point arm length for the OFF-to-ON transition. (FIG. 17D) Activity of the nano-chip with different ssDNA and dsDNA linkers. Under our experimental conditions, short dsDNA can be considered as a rigid rod with constant length, whereas the ssDNA linker can be considered as extendable soft linker. Therefore, to evaluate the effective sensor arm length, predicting the ssDNA effective length is the key consideration. To estimate the effective unit length of ssDNA, the inventors focused on the tipping point arm length for OFF-to-ON transition, as the data set obtained with different ssDNA/dsDNA combinations might show a similar activity profile along the estimated sensor arm length. With data set from different dsDNA linker length, the inventors estimated the tipping point arm length for each dsDNA linker length, and calculated the sum of difference of each tipping point arm length. The inventors estimated the empirical unit length of ssDNA (C_{ssDNA}) to be approximately 0.23 nm/nt because the corresponding data provided a minimum sum of the difference (inset, see methods). The data set from different dsDNA linker lengths was plotted using 0.23 and 0.34 nm/nt for ssDNA and dsDNA, respectively.

FIG. 18-1 shows the design of the ON switch. (FIG. 18A) Schematic drawing to explain the conformation change of the miRNA ON switch. Upon miRNA hybridization to the toehold region (“T” in the figure), miRNA invades the stem hybridized part of the switch (“H”), and replaces the short complementary part of the switch (“H*”). Therefore, the switch structure is converted to extended open form. L: Loop part (polyT) of the switch. h*: miRNA's complementary part to “H”. t*: miRNA's complementary part to “T”. (FIG. 18B) Representative structure of the let-7 miRNA ON switch. The inventors note that, to stabilize the sensor structure, additional hybridization pairs were introduced into the root of the hybridize region (see FIG. 18F). DG: Minimum free energy (MFE) of the sensor structure predicted by NUPACK at 1 M Na+ and 37° C. (FIG. 18C) Calculation of difference in effective sensor domain length (ΔL) upon activation. To simplify, the inventors set the 5′ end as an initiation point of miRNA hybridization. dsD/RNA: DNA-RNA hybrid double strand. (FIG. 18D) (upper) MFEs of the sensor structure at 37° C. in 1 M Na+ (black circle) and in the PURE system (50 mM Na+ and 18 mM Mg++, gray circle). Sensors having a 59 nt ssDNA domain and a 45 bp dsDNA domain were used. (middle) Relative activities of let-7 sensor with different hybridization part lengths (black circle: w/ miRNA, gray circle: w/o miRNA). The activities were normalized using the activity of a reference nano-chip at 90 min (also in FIGS. 18F and H). MFEs of the sensor structure calculated at 1 M Na+ were used (also in FIGS. 18F and H). For FIG. 18D, miRNAs at 100 nM were incubated with nano-chip for 30 min on ice, and then 3×excess volume of the PURE system solution was added to the reaction mixture. Therefore, the final miRNA concentration was 25 nM. (lower) ON/OFF ratio of each sensor. (FIG. 18E) To stabilize the sensor structure, additional hybridization pairs were introduced into the root of the stem structure. MFEs were as in FIG. 18D. (FIG. 18F) (upper) Relative activities of let-7 sensors with different hybridization part lengths (black circle: w/ miRNA, gray circle: w/o miRNA). The activities were normalized using the activity of a reference nano-chip at 90 min. MFEs of the sensor structure calculated at 1 M Na+ were used. For FIG. 18F, miRNAs at 100 nM were incubated with nano-chip for 30 min on ice, and then 3×excess volume of the PURE system solution was added to the reaction mixture. Therefore, the final miRNA concentration was 25 nM. (lower) ON/OFF ratio of each sensor.

FIG. 18-2 shows the design of the ON switch. (FIG. 18G) To evaluate the effect of effective loop length L′ (=T+L), the inventors changed the length L′ and measured the nano-chip activity. (FIG. 18H) (upper) MFEs of the sensor structure predicted by NUPACK at 1 M Na+ and 37° C. for an effective hybridization length H′=H+1=12 bp (black circle) and 11 bp (white circle). (lower) Relative activities of let-7 sensors with different effective loop lengths L′ (black circle: H′=12 and w/ miRNA, gray circle: H′=12 and w/o miRNA, white circle: H′=11 and w/ miRNA, light gray circle: H′=11 and w/o miRNA). The activities were normalized using the activity of a reference nano-chip at 90 min. For FIG. 18H, the final miRNA concentration was 25 nM. Main conclusion: Sufficient MEF (Minimum free energy) of the sensor structure was crucial to suppress the leak of the switch. And sufficient loop length L′ (and the total linker length) was crucial to fully activate the switch.

FIG. 19 shows the kinetic analysis of miRNA hybridization to the nano-chip. (FIG. 19A) Image of 1% Agarose gel (1 mM Mg++ inside the gel). The reaction times were 60, 300 and 1,800 s (1, 5 and 30 min) at 37° C. The inventors note that the nano-chip was used without purification to remove the unbound freely diffusing substrate-gene. The remaining unbound substrate-gene was used as a control. (FIG. 19B) (upper) Quantification of the data in a. The inventors quantified the bound miRNA using the calibration lane in the same gel (not shown). (lower) The estimated association constant kₒₙ was 3.4×10⁵ [/M/s] and 2.8×10⁵ [/M/s] for nano-chip and freely diffusing substrate-gene, respectively, indicating that the miRNA kₒₙ to the sensors integrated on nano-chip was in the similar order of that to the freely diffusing sensors. (FIG. 19C) Apparent Km value of let-7 sensor was measured. For FIG. 19C, miRNAs were directly added into the PURE system solution containing nano-chip, and incubate 30 min on ice before initiate the transcription-translation reaction.

FIG. 20 shows the effect of mismatch on let-7 ON switch. (FIG. 20A) To examine the effect of mismatches (small letter), miRNA was used with a single let-7 ON switch (hybridization part=12 nt). MFEs, as predicted using NUPACK, of the sensor structure at 37° C. and 1 M Na+. (FIG. 20B) Comparison of the experimental data with the predicted values. (upper) Comparison of the predicted binding ratios (black line) with the experimental data (white bar) to estimate the effect of mismatch position. (lower) Comparison of the predicted values with the experimental data to evaluate the relationship between these two parameters, showing that above a 15% binding ratio, the mismatched position could activate the chip fully. For FIG. 20B, miRNA was directly added into the PURE system solution containing the nano-chip, and incubated 30 min on ice before initiating the transcription-translation reaction. (FIG. 20C) Pseudo-color display of the experimental data. The let-7 miRNAs target site was denoted with a black circle.

FIG. 21 shows Orthogonality of the ON switch. (FIG. 21A) Sensor structure sequences and MFEs at 37° C. (FIG. 21B) Representative data of the let-7 sensor's orthogonality. (FIG. 21C) Matrix data of the crosstalk between eight sensors. The activities were normalized using the activity of a reference nano-chip at 90 min. Data from three independent experiments are shown (mean±standard deviation). For FIG. 21B, C, miRNAs, except miR-224-5p, at 100 nM were incubated with nano-chip for 30 min on ice, and then 3 x excess volume of the PURE system solution was added to the reaction mixture. Therefore, the final miRNA concentration was 25 nM. For miR-224-5p, the final miRNA concentration is 100 nM. (FIG. 21D) 5′ identical nucleotides of miR-1-3p and miR-206 slightly affect on the orthogonality of the switches. (FIG. 21E) miR-224-5p self-dimerizes under the test conditions, which might produce the low response ability of the miR-224-5p sensor.

FIG. 22 shows the design of the OFF switch. (FIG. 22A) Schematic drawing to explain the switching mechanism. Upon miRNA hybridization, the sensor structure was stabilized, and the folded form became dominant, shortening the effective reach length of the sensor arm and switching off the activity of nano-chip. (FIG. 22B) Representative secondary structure of the OFF switch predicted using NUPACK (number of supported hybridization pairs is 6 bp). (FIG. 22C) Stability of the sensor alone (upper) and sensor-miRNA (lower) of the OFF switch. MFEs, as predicted using NUPACK, of the structure at 23° C. (not at 37° C.) in 1 M Na+ (black circle) and in the PURE system (50 mM Na+ and 18 mM Mg++, gray circle). (FIG. 22D) (upper) Relative activities of the let-7 OFF sensor with different supported hybridization part lengths. The activities were normalized using the activity of a reference nano-chip at 90 min. The inventors note that the measurements were performed at 23° C. (not at 37° C.). For FIG. 22D, miRNAs at 100 nM were incubated with nano-chip for 30 min on ice, and then 3×excess volume of the PURE system solution was added to the reaction mixture. Therefore, the final miRNA concentration was 25 nM. (lower) ON/OFF ratio of each sensor. (FIG. 22E) Measured apparent Kᵢ value of let-7 OFF switch. For FIG. 22E, miRNA was directly added into the PURE system solution containing the nano-chip, and incubated 30 min on ice before initiating the transcription-translation reaction.

FIG. 23 shows the design of the 2-input AND switch. (FIG. 23A) Schematic drawing to explain the extension of the sensor end-to-end distance. Upon miRNA hybridization, the stem-loop structure was extended. (FIG. 23B) Schematic drawing to explain the switching mechanism. Upon hybridization with two different miRNAs, the reach length of the sensor arm is of sufficient length to activate the nano-chip. (FIG. 23C) Predicted secondary structure of the AND switch at 37° C. and 1 M Na+ using NUPACK. (FIG. 23D) Bulk experimental activity of AND switch. The nano-chip concentration is approximately 1 nM. For FIG. 23D, miRNAs at 400 nM were incubated with nano-chip for 30 min on ice, and then 3×excess volume of the PURE system solution was added to the reaction mixture. Therefore, the final miRNA concentration was 100 nM. (FIG. 23E) Single AND-chip activity in water-in-oil (w/o) droplets. The nano-chip concentration is 0.4 pM. For FIG. 23E, miRNA was directly added into the PURE system solution containing the nano-chip, and incubated 30 min at 20° C. (time required to form water-in-oil (w/o) droplet) before initiating the transcription-translation reaction.

FIG. 24 shows the design of the 3-input AND switch. (FIG. 24A) Schematic drawing to explain the extension of the sensor end-to-end distance. Upon miRNA hybridization, the stem-loop structure was extended. (FIG. 24B) Predicted secondary structure of the 3-input AND switch for let-7/miR-206/miR-92a-3p at 37° C. and 1 M Na+ using NUPACK. (FIG. 24C) Bulk experimental results of the 3-input AND switches for let-7/miR-206/miR-197-3p (upper) and let-7/miR-365a-3p/miR-183-5p (lower). (FIG. 24D) Single 3-input AND-chip activity in water-in-oil (w/o) droplets. The nano-chip concentration was 0.4 pM. For FIG. 24C, miRNA was directly added into the PURE system solution containing the nano-chip, and incubated for 30 min at 20° C. (time required to form water-in-oil (w/o) droplet) before initiating the transcription-translation reaction.

FIG. 25 shows the functional modulation of the 3-input switch by changing the molecular layout. (FIG. 25A) Conversion from AND switch to OR switch. The difference is two inputs minus the thickness of two stem-structures (22 nt×2×0.34 nm/nt+22 nt×2×0.23 nm/nt−4˜21 nm). Therefore, the inventors changed the intermolecular distance between the RNAP enzyme and substrate-gene from 50 to 70 nm. (FIG. 25B) Conversion from AND switch to Majority switch. The inventors changed the dsDNA linker length from 45 to 85 bp, a difference approximately equivalent to one input minus the thickness of one stem-structure (22 nt×1×0.34 nm/nt+22 nt×1×0.23 nm/nt−2˜11 nm˜31 nt dsDNA).

FIG. 26 shows the nano-chip activity in the PURE system. (FIG. 26A) Effect of nano-chip concentration. The activities were normalized using the value for 0.52 nM nano-chip at 180 min. (FIG. 26B) Effect of miRNA concentration. The fluorescence intensities were normalized using the value for 0 nM miRNA at 180 min. (FIG. 26C) Effect of the miRNA addition time on the ON switch integrated nano-chip activity. The arrow indicates the miRNA addition time (final miRNA concentration of 50 nM). The final nano-chip concentration is 0.13 nM. The activities were normalized using the activity of a reference nano-chip at 180 min. Dashed, black and gray lines indicate the nano-chip activity, where the dashed line indicates the inactive nano-chip, the black line indicates the sensor-activated but sfGFP mRNA production-inactivated nano-chip, and the gray line indicate the sensor- and mRNA production-activated nano-chip. (FIG. 26D) Effect of other chip activity on the sensor-integrated nano-chip. The arrow indicates the timing of miRNA addition (final miRNA concentration of 50 nM). The final nano-chip concentration for both nano-chip is 0.13 nM. The activities were normalized using the activity of a reference nano-chip at 180 min. Dashed, black, gray and dark gray lines indicate the nano-chip activity, where dashed line indicates the inactive nano-chip, the black line indicate the sensor-activated but sfGFP mRNA production-inactivated nano-chip, and gray line indicate the sensor- and mRNA production-activated nano-chip, dark gray line indicate the sfCherry mRNA production-activated nano-chip. The decrease of the sfGFP production might be explained by the shortage of protein production resources (e.g., the PURE system components). Main conclusion: Lower nano-chip concentration (˜0.5 nM) is crucial to prevent the resource consumption. miRNA activation timing is important for the miRNA-sensing-chip activity, if the other chip was functioning and thus the transcription-translation system were operating in the background.

FIG. 27 shows that nano-chip communication enables genetic circuits. (FIG. 27A) Schematic drawing of the genetic circuit comprising of two “buffer (ON switch)” function nano-chips. (FIG. 27B) Activities of the circuit shown in (A). (FIG. 27C) Schematic drawing of the genetic circuit consisting of two AND switch nano-chips. (FIG. 27D) Leak check of the circuit shown in (C). (FIG. 27E) Chip 1 concentration affects the overall production of the genetic circuit shown in (C). (FIG. 27E) Lag time of sfGFP protein production in the control reaction. (FIG. 27G) Kinetic analysis of the genetic expression shown in (F). Dots indicate the experimental data and the fitting lines indicate the predicted time evolution using the estimated transcription rates: 0.034 and 0.02/sec/molecule of substrate-gene for the nano-chip-initiated (RNAP at the same concentration as the nano-chip) and DNA-initiated (RNAP˜30 nM, a much greater than that of the nano-chip) reaction, respectively. At a high RNAP concentration, the experimental value is lower than the predicted one, which may reflect the resource consumption of the gene expression system.

FIG. 28 shows that nano-chip base genetic circuit can detect and respond to the miRNA profile of artificial cells. Region of interest (ROI) indicate the position shown in FIG. 4F.

FIG. 29 shows the design of the LacI switch. (FIG. 29A) (upper) Three-dimensional protein-nucleic acid structure of LacI-DNA (PDB: 1LBG) drawn by iCn3D (https://www.ncbi.nlm.nih.gov/Structure/icn3d/icn3d.html). LacI tetramer binds to two DNA recognition sites. (lower) Two LacO sequences were used in the experiment. (FIG. 29B) (upper) Schematic drawing of the LacI sensor. P: promoter, RBS: ribosome binding site, ORF: open reading frame, T: terminator. (lower) To obtain the ON and OFF switches, the same sensor was anchored to different positions on the DNA origami. The inter-molecular distance between the anchor positions of the enzyme (T7 RNAP, black circle) and substrate gene (LacI sensor, black square) is 32 and 70 nm for the ON and OFF switches, respectively. (FIG. 29C) Schematic drawing to explain the switching mechanism. Upon LacI tetramer binding, the reach length of the sensor arm is sufficiently short to activate the nano-chip. (FIG. 29D) LacI sensor activity in the nano-chip (upper) and in the reaction-diffusion system (freely diffusing LacI sensor reacted with freely diffusing LacI-T7 RNAP in solution). Gray circle: without (w/o) IPTG, black circle: with (w/) 100 mM IPTG. Activities were normalized using the data at 180 min (0 nM LacI w/o IPTG was set to 1). (FIG. 29E) Schematic drawing to explain the switching mechanism the LacI OFF switch. Upon LacI tetramer binding, the reach length of the sensor arm is too short to activate the nano-chip. (FIG. 29F) Activity of the LacI OFF switch.

FIG. 30 shows the design of the Ampicillin (Amp) switch. (FIG. 30A) Predicted structure of Amp aptamer. (FIG. 30B) Schematic drawing to explain the switching mechanism the Amp OFF switch. Upon Amp binding, the reach length of the sensor arm is not enough long to fully activate the nano-chip. (FIG. 30C) Activity of the Amp OFF switch.

FIG. 31 shows the design of NOT switch (NAND-type). (FIG. 31A) Logic operation of NAND switch. (FIG. 31B) Schematic drawing to explain the switching mechanism. Upon hybridization with miRNAs, the reach length of the sensor arm is too long, and thus inactivate the nano-chip. (FIG. 31C) Activity of the NAND switch.

FIG. 32 shows the switching of the activity through anchor point arrangements. (FIG. 32A) Schematic drawing to explain the switching mechanism of the IPTG ON switch. Upon IPTG binding, the reach length of the sensor arm is enough long to activate the nano-chip. (FIG. 32B) Schematic drawing to explain the switching mechanism of the IPTG OFF switch. Upon IPTG binding, the reach length of the sensor arm is too short to activate the nano-chip.

FIG. 33 shows the photoswitching of nano-chip activity by UV irradiation. (FIG. 33A) P, promoter; cnvK, photoreactive crosslinking nucleotide. 5 min irradiation of 366 nm UV light converted the cnvK UV sensor to the short form, resulting in the switching off of the nano-chip activity (operation #1). Further 10 min irradiation of 312 nm UV light resume the nano-chip activity (operation #2). (FIG. 33B) On/off switching by UV irradiation.

FIG. 34 shows the genetic circuits responding to UV input and miRNA profile. (FIG. 34A) (upper) In the circuit, in the absence of UV irradiation (365 nm), chip 1 produces the communication transmitter miRNA (miR-206). Chip 2 produces the sfGFP mRNA only when all of let-7, miR-92a-3p and transmitter miRNA (miR-206) exist. (lower) Logic function of the circuit. (FIG. 34B) (upper) In vitro sfGFP expression upon UV input and miRNA profile. (lower) Relative value of the data (two miRNAs w/o UV was set to 1).

FIG. 35 shows the design of the nano-chip functioning in individual system (living organism). (FIG. 35A) Schematic illustration of the molecular layout of RNAP and target gene is inside nano-cage. (FIG. 35B) Schematic illustration of the experiment. About 1 uL of nano-chip (˜5 nM) was microinjected into the zebrafish embryo (fertilized and unfertilized rabbit ova). After 1-hour incubation at 28° C., RNAs were extracted from the embryo through Trizol and DNaseI treatment. Then, product RNA was quantified using real-time qRT-PCR assay. (FIG. 35C) Relative values of product RNA (nano-chip having both RNAP and target gene was set to 1).

### DESCRIPTION OF EMBODIMENTS

Followings are explanations of the present invention. Unless otherwise stated, the terms used in the present specification have the same meaning as those generally used in the pertinent field.

### Devices for Genetic Circuits

In the present invention, the “catalyst” means a material that accelerates or inhibits a chemical reaction such as biocatalyst and inorganic catalyst. In concrete terms, such catalyst includes enzyme, protein, nucleic acids and artificial nucleic acids and the like. Specifically, DNA-/RNA-/artificial nucleic acids-polymerase, polymer synthetase, nucleic acids binding proteins (including, but not limited to, restriction enzyme, ligase, nickase, methylase, transcription factor), molecular chaperons (including, but not limited to, HSP70 and HSP90), molecular motor (including, but not limited to, kinesin, myosin and dynein), membrane proteins, modification enzymes (including, but not limited to, phosphorylation kinase and ubiquitin ligase), synthetase (including, but not limited to, F1-ATPase), degradation enzyme (including, but not limited to, protease and proteasome), RNA enzyme (including, but not limited to, ribozyme and Long non-coding RNA (lncRNA)), DNA enzyme, artificial nucleic acid enzyme and nucleic acid-protein complex (including, but not limited to, ribosome including mutant that can incorporate unnatural amino acids), RNA-inducing silencing complex (RISC) and CRISPR/Cas)) and the like. In the present specification, the “artificial nucleic acids-polymerase” represents a polymerase that uses following nucleic acids as substrates: artificial nucleic acids, or both of artificial nucleic acids and natural nucleic acids, or chemically modified artificial/natural nucleic acids (V B Pinheiro et al., Science 336, 341-344 (2012); A I. Taylor et al., Nature 518, 427-430 (2015); N. Ramsay et al., JACS 132, 5096-5104 (2010)); the “polymer synthetase” represents an enzymes that synthesizes high molecular weight polymer (e.g., bioplastic) or materials thereof (B H. Rehm, Nat Rev Microbiol. 8, 578-92 (2010); T. Iwata, Angew Chem Int Ed Engl. 54, 3210-3215 (2015)). The enzymes and proteins used in the present invention can be prepared through a method known per se using a recombinant protein expression system.

The above catalyst can be attached to the shape change element (described below), or can be separated from the shape change element. When the catalyst is separated from the shape change element, the catalyst can be attached to a base member.

In the present invention, the “target gene” means a gene being induced the expression thereof upon making contact with above catalyst. In concrete terms, examples are sfGFP gene and small RNA genes and the like. The target gene can contain any number of regulator sequences (including, but not limited to, transcription regulator, translation regulator, promoter, ribosome binding region, enhancer and replication origin) and any number of other factors (including, but not limited to, selector gene, signal peptide, code sequence for tag-peptide and terminator) and the like. These regulator sequences and other factors can operably be linked to the target gene as well known in the technological field of the present invention.

In the present specification, the “promoter” can contain regions to which an RNA polymerase and other transcription factors. In the present specification, gene expression can mean as protein synthesis through transcription and translation, or as RNA synthesis in the cases of a gene functioning as RNA (for example non-coding RNA). The above target gene can be attached to the below-mentioned shape change element, or can be separated from the shape change element. If the target gene is separated from the shape change element, the target gene can be attached to the base member.

Genetic circuit devices of the present invention, can be used as the “switch”. In the present invention, the “switch” relates to gene expression control, and has at least one “open-close mechanism”. The “open-close mechanism” has shape change element as a driving part. The shape change element has the first site and the second site, and the distance between the first and second sites are changed by the action of activation source. The first and second sites can be positioned at any part of the shape change element: for example, the both ends of the shape change element. If the shape change element has the first anchor part at the first site and the second anchor part at the second site and the catalyst and the target gene bind to the shape change element, the catalyst and the target gene are fixed on these anchor parts. The first site and the first anchor part can be the same site/part or can be the different site/part. Similarly, the second site and the second anchor part can be the same site/part or can be the different site/part.

The material of the shape change element of the present invention is not limited, if the length can be defined. In concrete terms, the shape change element includes protein/peptide chain, nucleic acid (DNA, RNA and artificial nucleic acid), protein-nucleic acid complex, metal material (piece of metal, gold nano-rod), semiconductor (silicon), carbon allotrope (CNT, graphite, fullerene, diamond), inorganic material (ceramic), non-crystalline solid (glass), organic material, high molecular weight polymer.

In the present invention, the “linker”, the “anchor arm” the “sensor” and the “sensor arm” correspond to the “shape change element”. Additionally, in the present specification, the “linker”, the “anchor aim”, “sensor” and “sensor arm” can be used interchangeably. Moreover, the shape change element may be configured to two parts: one part permitting change of the shape by an action of the activation source (for example, referred to as the “sensor part”) and the other part (for example, referred to as the “arm part”).

In the present specification, the size (length) of the shape change element is defined as follows: if a binding partner of the shape change element is a target gene, the length thereof is the length between the fixed position of the shape change element in the base member and the promoter sequence; if a binding partner of the shape change element is a catalyst, the length thereof is the length between the fixed position of the shape change element in the base member and the position of the catalyst. Additionally, in the present specification, the fixed part, where the shape change element is fixed in the base member, may be referred to as a fixed end.

The Open-close mechanism has the shape change element as a movable part, and is configured to perform an open-close motion in which the target gene and the catalyst relatively approach and contact with each other or relatively leave the contact state in response to a change in the distance between the first site and the second site in the shape change element. Additionally, as long as taking the above configuration, the open-close mechanism can contain any constituent element.

The whole part of the open-close mechanism can be the shape change element, or the part of the open-close mechanism can be the shape changing element.

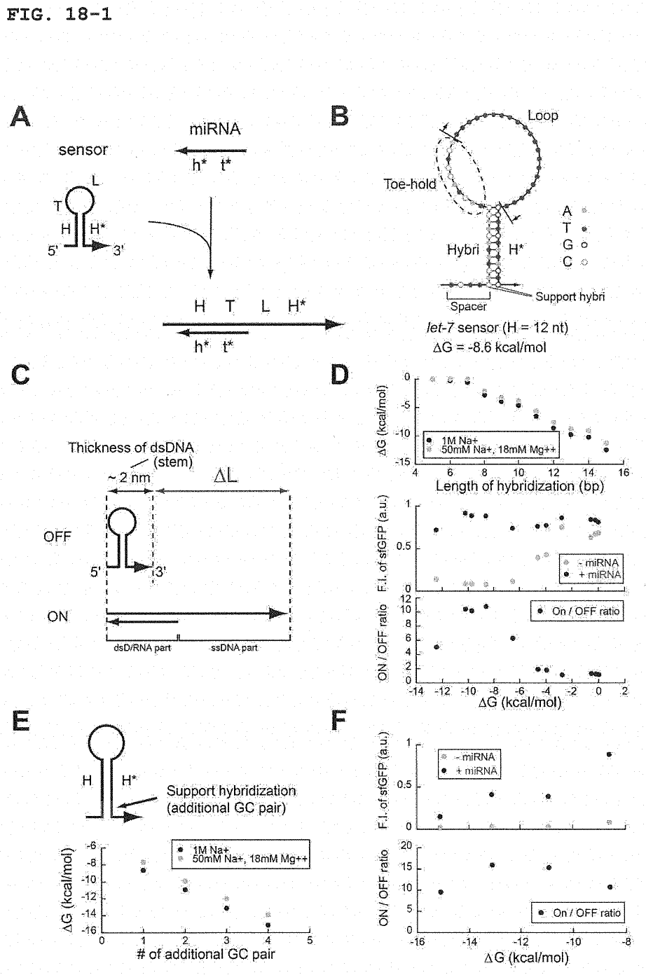

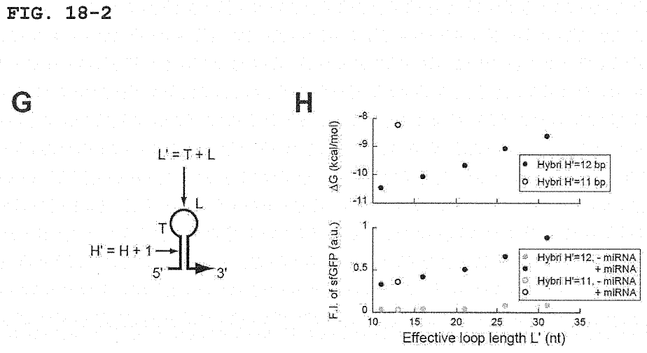

A specific structure of the shape change element includes, for example, the stem-loop structure. The basic structure of the stem-loop, contains five domains: toehold, hybridization, loop, anti-hybridization and the spacer between the stem-loop and the other part of the linker (FIG. 18B). MicroRNA (miRNA), which binds to the Toehold and Hybridization domains, first binds to Toehold domain and then penetrates into the stem structure and finally unfolds the stem-loop. The “support hybridization” can be further introduced at the root of the stem structure to support stable hybridization.

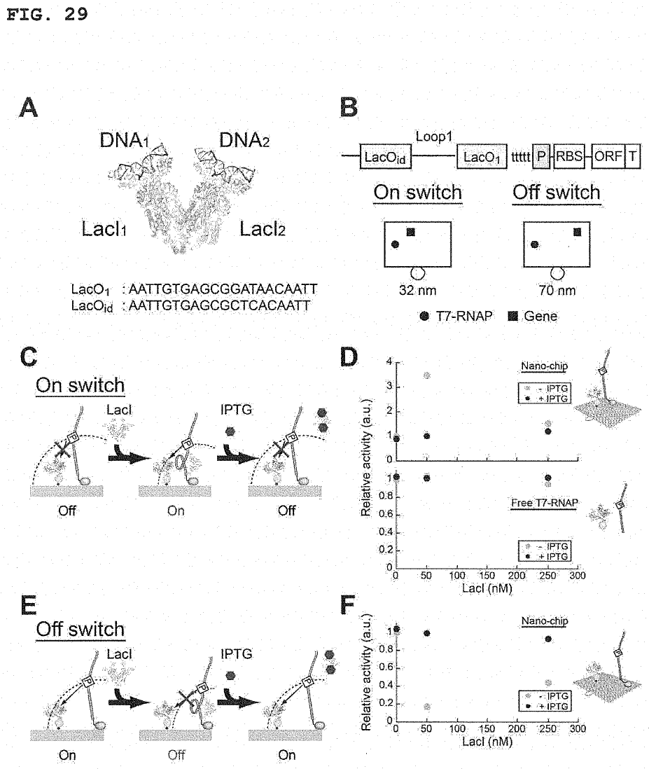

Another specific structure of the shape change element includes the shape change element utilizing a double-stranded DNA using a LacI-LacO pair (FIG. 29A-D). LacI, a dsDNA binding protein that forms a tetramer, can bind to two LacO sequences (FIG. 29A). Firstly, LacI, which is the first activation source, binds to two LacO sequence, folding the dsDNA and shortening the dsDNA length. Further addition of isopropyl β-D-1-thiogalactopyranoside (IPTG) induces the release of LacI from the LacO sequence, resulting in the opening of the dsDNA to its original length (FIGS. 29C and E).

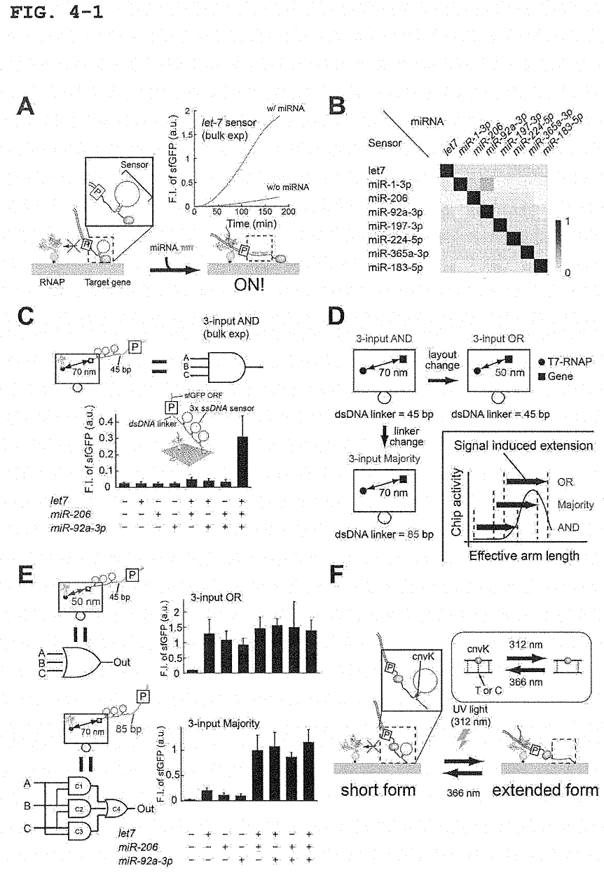

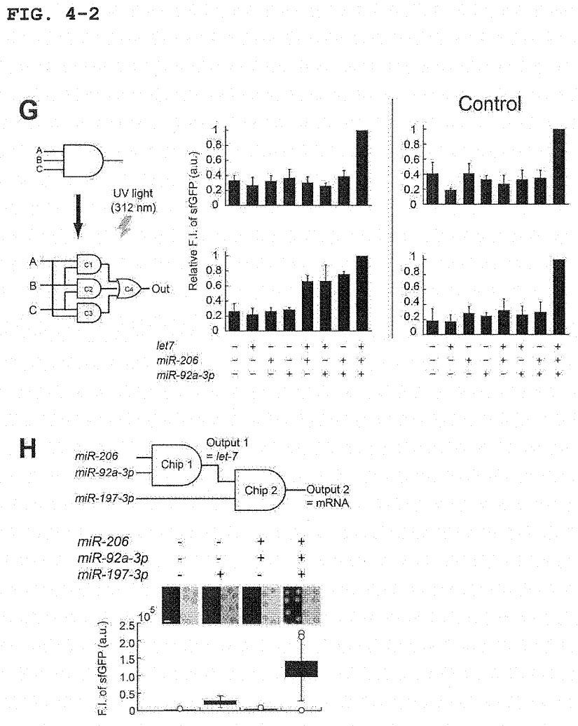

Another specific structure of the shape change element includes the shape change element utilizing photoreactive cross-linking nucleotides cnvK (3-cyanovinylcarbazole nucleoside) (FIG. 4F). With UV irradiation, cnvK can make a covalent bond with the pyrimidine (T or C) of the complementary strand. In the shape change element using cnvK, firstly, the first activation source (366 nm UV light) induces the cross-link to fold the linker nucleotides between the base member (DNA origami) and StreptAvidin for fixing the target gene and to shorten the length thereof, whereas additional ultraviolet light (312 nm) of the second activation source irradiation promotes the reverse reaction, opening of the linker to its original length.

Among the switches of the present invention, the switch which is configured such that the catalyst and the target gene relatively approach and contact with each other by an action of the activation source is referred to as “ON switch”. Upon ON switch inputted, the frequency of contact of the target gene and the catalyst increases than that before the input, resulting in the increase of the target gene expression greater than that of background.

On the other hand, among the switches of the present invention, the switch which is configured such that the catalyst and the target gene relatively leave the contact state thereof by an action of the activation source is referred to as “OFF switch”. Upon OFF switch inputted, the frequency of contact of the target gene and the catalyst decreases that that before the input, resulting in the decrease of the target gene expression to the level similar to that of background.

Specific structure and mechanism (switching mechanism) of the present invention include a structure or mechanism, for example, comprising:
(i) a first shape change element as a mutually independent first movable part, and a second shape change element as a mutually independent second movable part, in which the catalyst is fixed on the side of a first site of the first shape change element in the first movable part, in which the target gene is fixed on a first site side of the second shape change element in the second movable part, in which the second site side of each shape change element in the first and the second movable parts is fixed (e.g., separately from each other on one plane of) the base member, in which the open-close mechanism is configured such that the aforementioned catalyst and the target gene relatively approach and contact with each other or relatively leave the contact state by an increase in the distance between the first site and the second site in each shape change element, and in which the open-close mechanism is configured such that the catalyst and the target gene relatively approach and contact with each other or relatively leave the contact state by a decrease in the distance between the first site and the second site in each shape change element, or
(ii) either one of the catalyst and target gene is fixed on (e.g., one plane of) the base member, and the other is fixed on the side of the first site of the shape change element in the movable part, in which the side of the second site of the shape change element in the movable part is fixed on (the plane of) the base member, in which the open-close mechanism is configured such that the catalyst and the target gene relatively approach and contact with each other or relatively leave the contact state by an increase in the distance between the first site and the second site in the shape change element, and in which the open-close mechanism is configured such that the catalyst and the target gene relatively approach and contact with each other or relatively leave the contact state by a decrease in the distance between the first site and the second site in the shape change element.

The switches of the present invention include the structure and mechanism (switching mechanism) where the base member has flexibility. Specific structure and mechanism (switching mechanism) include a structure or mechanism, for example, in which the catalyst and the target gene are placed (separately from each other) on (one of the planes) of the (sheet-like) base member, in which the first site and the second site of the shape change element are fixed (separately from each other on the same plane) of (the sheet-like) base member, in which a decrease or an increase in the distance between the first site and the second site deforms (the sheet-like) base member, and in which the catalyst and the target gene relatively approach and contact with each other or relatively leave the contact state by the deformation of (the sheet-like) base member.

In the present invention, the “base member” is not particularly limited, if the “base member” can fix the molecules such as the catalyst, the target genes and the shape change elements and the like and has a plane in which the molecules can fix at a desired position. The “base number” can also have flexibility. In the present specification, the site on the base member to which the catalysts, target genes and shape change elements and the like are fixed may be referred to as the anchor part. If there are several anchor parts, to distinguish each other, each anchor part can be referred to as the first-, the second- and the third anchor part and so on. The form of the base member can be belt-shaped, sheet-like, string, linear and or massive/aggregated. Base members include such as, but not limited to, nano structure composed of DNA, RNA, artificial nucleic acids, protein and polymer and the like. In concrete base members include such as DNA-, RNA-, peptide-, protein-origami and supramolecular polymer.

In the present specification, the “base member” and the “scaffold” can be used interchangeably.

In the present invention, “the AND switch” means the element that has 2 or more inputs (terminals) and one output (terminal), which outputs “1” to output (terminal) when input “1” is input to all the inputs (terminals), whereas output “0”, if inputs “0” is input to at least one of the input (terminal). The “input” means, for example, the state of the shape change element becomes driven by an action of the activation source. “Output” means, for example, expression of the target gene.

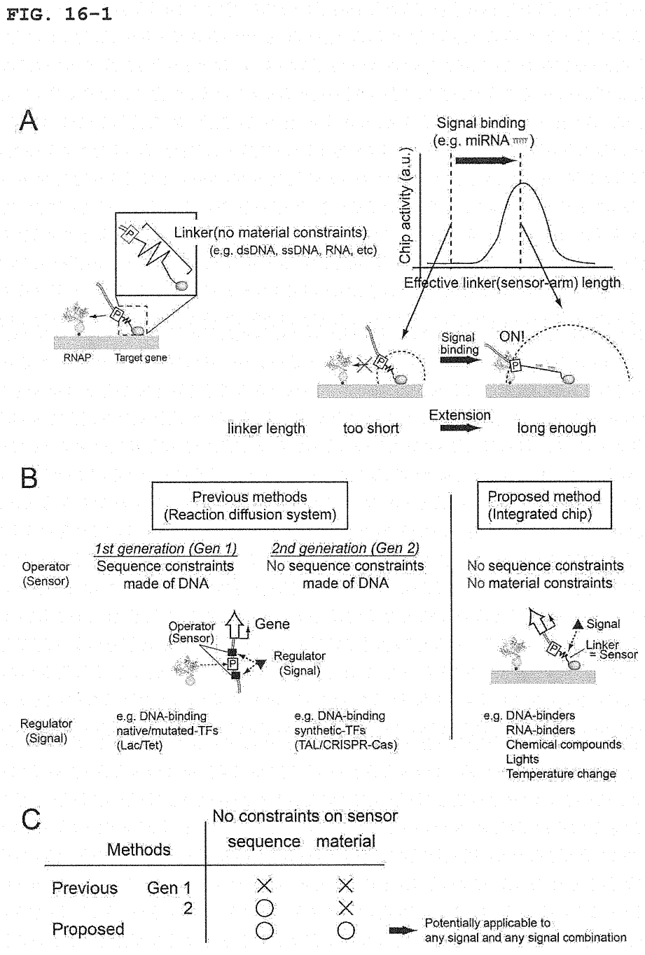

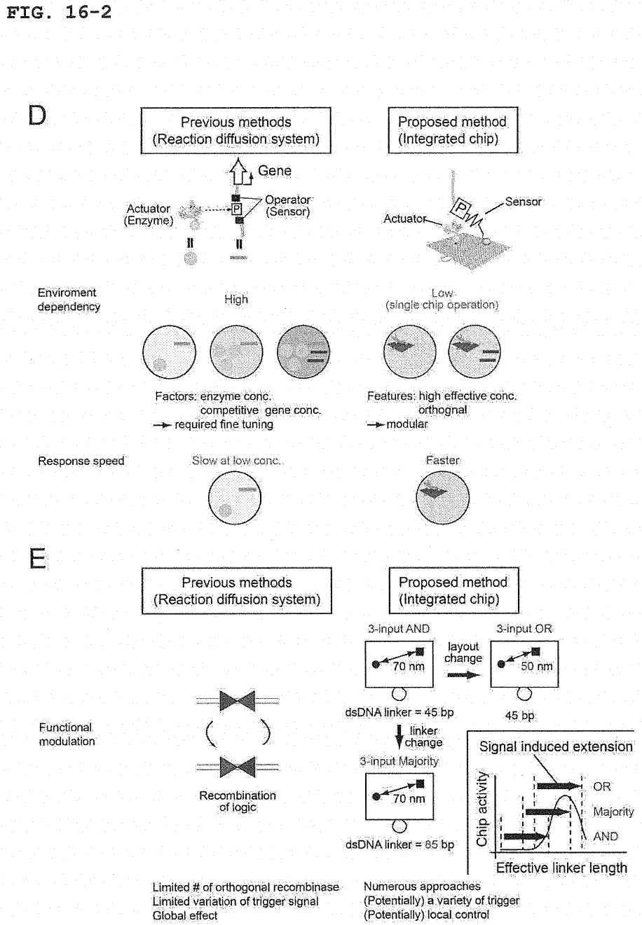

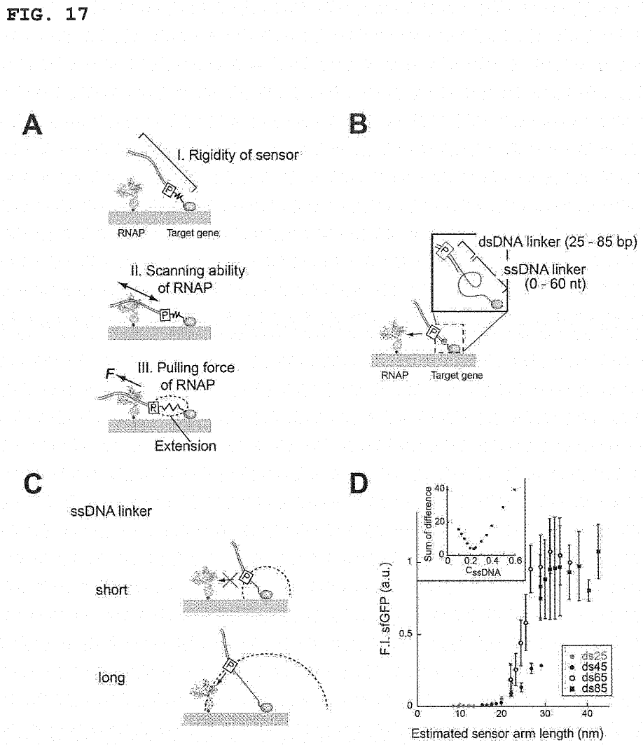

The AND switch can be constructed by connecting several (for example, 2, 3 or more) shape change elements directly to induce, in the case of all the inputs (terminals) receive “1”, sufficient extension of the effective linker length to cross the tipping point (FIGS. 4D and 16E). The coupling scheme of the shape change element is not particularly limited, if the coupling scheme allows the distance change of each shape change element. Such coupling scheme includes, for example, tandem and parallel and the like. In the present specification, the “tipping point” means the specific linker length where the action of the activation source induces the change of the shape change elements, and thus the transition of the catalytic activity from inactive-to-active or active-to-inactive is occurred (FIG. 17).

In the present specification, the AND switches performing the logic operation as described above and the “AND gate” can be used interchangeably.

In the present invention, the “OR switch” means the element that has 2 or more inputs (terminals) and one output (terminal), which outputs “1” to output (terminal) when input “1” is input to at least one of the input (terminal), whereas outputs “0”, if input “0” is input to all of the inputs (terminals).

The OR switch can be constructed by connecting several (for example, 2, 3 or more) shape change elements directly to induce, in the case of at least one of the input (terminal) receive “1”, sufficient extension of the effective linker length to cross the tipping point (FIGS. 4D, 16E and 25). The coupling scheme of the shape change element is not particularly limited, if the coupling scheme allows the distance change of each shape change element. Such coupling scheme includes, for example, tandem and parallel and the like.

In the present specification, the OR switches performing the logic operation as described above and the “OR gate” can be used interchangeably.

In the present invention, the “MAJORITY switch” means the element that has 3 or more inputs (terminals) and one output (terminal). Based on the majority rule, the “MAJORITY switch” outputs “1” to output (terminal), when input “1” is input to majority of the input (terminal), whereas outputs “0”, if input “0” is input to majority of the inputs (terminals).

The MAJORITY switch can be constructed by connecting several (for example, 2, 3 or more) shape change elements directly to induce, in the case of the majority input (terminal) receive “1”, sufficient extension of the effective linker length to cross the tipping point (FIGS. 4D, 16E and 25). The coupling scheme of the shape change element is not particularly limited, if the coupling scheme allows the distance change of each shape change element. Such coupling scheme includes, for example, tandem and parallel.

In the present specification, the MAJORITY switches performing the logic operation as described above and the “MAJORITY gate” can be used interchangeably.

In the present invention, the “NOT switch” means the element that has one input (terminal) and one output (terminal), which outputs “0” to output (terminal), when input “1” is input to the input (terminal). The NOT switch can be constructed by designing the chip to induce, in the case of input (terminal) receives “1”, sufficient extension of the effective linker length to cross the tipping point.

In the present specification, the NOT switches performing the logic operation as described above, “NOT gate”, “OFF switch” and “OFF gate” can be used interchangeably.

In the present invention, the “NOR switch” means the m element that has 2 or more inputs (terminals) and one output (terminal), which outputs “0” to output (terminal) when input “1” is input to at least one of the input (terminal), whereas outputs “1”, if input “0” is input to all of the inputs (terminals).

The NOR switch can be constructed by connecting several (for example, 2, 3 or more) shape change elements directly to induce, in the case of at least one of the input (terminal) receives “1”, sufficient extension of the effective linker length to cross the tipping point (FIGS. 4D, 16E). The coupling scheme of the shape change element is not particularly limited, if the coupling scheme allows the distance change of each shape change element. Such coupling scheme includes, for example, tandem and parallel.

In the present specification, the NOR switches performing the logic operation as described above and the “NOR gate” can be used interchangeably.

In the present invention, the “NAND switch” means the element that has 2 or more inputs (terminals) and one output (terminal), which outputs “0” to output (terminal) when input “1” is input to all the inputs (terminals), whereas outputs “1”, if input “0” is input to at least one of the input (terminal).

The NAND switch can be constructed by connecting several (for example, 2, 3 or more) shape change elements directly to induce, in the case of all the inputs (terminals) receive “1”, sufficient extension of the effective linker length to cross the tipping point (FIGS. 4D and 16E). The coupling scheme of the shape change element is not particularly limited, if the coupling scheme allows the distance change of each shape change element. Such coupling scheme includes, for example, tandem and parallel.

In the present specification, the NAND switches performing the logic operation as described above and the “NAND gate” can be used interchangeably.

In the present specification, the base member that is integrated with at least one switch described above is referred to as the “chip”. In the present specification, the “chip,” “nano-chip” and “gene nano-chip” can be used interchangeably. Additionally, the devices for gene circuit of the present invention itself may be the above switch.

The switches of the present invention (AND-, OR- and MAJORITY-switch) can be changed the logic function in a nanochip having a different structure through the approach controlling the intermolecular distance between the catalyst and the target gene used in the present invention: change of the molecular layout (anchor part) thereof, change of the shape of the base member, or change of the linker length. Specifically, for example, reprogramming of the logic function can be achieved by introducing the artificial nucleic acids, which can be switched between two states (cross-linking and separation (cleavage)) by light irradiation of different wavelengths, into a part of the stem-loop structure, or into a linker part between the base member and the gene. Additionally, for example, reprogramming of the logic function can be achieved by the activation source dependent separation (or binding) between any fixed part of the shape change element, which exists in-between the first- and the second-part of shape change element, and the anchor part on the base member.

In addition, genetic circuits can be prepared by integrating the switches (combination of AND-, OR-, MAJORITY-, NOT-, NOR- and/or NAND-switch) in the base member. Such genetic circuits can be prepared by integrating several switches on the base member, or by combining at least two chips described above.

### Integration on Base Member

In the present invention, the methods for integrating the target gene, the catalyst and the shape change element on the base membrane include, but are not limited to, for example, hydrogen bond such as hybridization of the nucleic acids, stacking interaction between nucleic acids, non-covalent bond such as between avidin-biotin, covalent bond such as between SNAP protein and SNAP ligand, and the combination of above interaction and the like. Specifically, for example, if the base member is DNA origami, the target gene and the shape change element (e.g., nucleic acids) can be modified with biotin through a method known per se, and the DNA origami can be attached streptavidin through a method known per se. Then, the target gene and the shape change element can be integrated by avidin-biotin interaction. Additionally, the catalyst (e.g., protein) can be expressed through a method known per se as fusion protein to SNAP-tag, and then can be integrated on DNA origami through a method known per se.

### Promoter

In the present invention, promoters are not particularly limited, if the promoters can express the target gene in the environment, in which the gene circuit devices of the invention are used. Promoter can be inducing- and constitutive-promoter.

Specifically, for example, but are not limited to, a promoter derived from cytomegalovirus (CMV, e.g., cytomegalovirus early-immediate promoter), human immunodeficiency virus (HIV, e.g., HIVLTR), Rous sarcoma virus (RSV, e.g., RSV LTR), murine mammary tumor virus (MMTV, e.g., MMTV LTR), Moloney murine leukemia virus (MoMLV, e.g., MoMLV LTR), herpes simplex virus (HSV, e.g., HSV thymidine kinase (TK) promoter), SV40 (e.g., SV40 early promoter), Epstein-Barr virus (EBV), adeno-associated virus (AAV, e.g., AAV p5 promoter), adenovirus (AdV, Ad2 or Ad5 major late promoter) can be used.

In addition, for example, but are not limited to, a promoter of polyhedrin, P10, trp, lac, recA, λPL, lpp, T7, SPO1, SPO2, penP, PHO5, PGK, CAP, ADH can be used.

Furthermore, for example, but are not limited to, a promoter of U6, H1, tRNA can be used.

### Signal Peptide

In the present invention, signal peptides include, but are not limited to, Golgi body-, cell membrane-, mitochondria-, nuclear-, synapse-, nucleolus-, nuclear membrane- and peroxisome-localization signal peptide and the like.

### Tag Peptide

In the present invention, tag peptides include, but are not limited to, FLAG tag peptide, HA tag peptide, MYC tag peptide, GFP tag peptide, MBP tag peptide, GST tag peptide, HIS tag, SNAP tag, ACP tag, CLIP tag, TAP tag and V5 tag and the like.

### Terminator

In the present invention, terminators are not particularly limited, and include any sequences generally known to a person skilled in the art. Specifically, terminators include terminator sequences derived from virus genome, mammals and birds. More specifically, terminators include, for example, but are not limited to, bovine growth hormone terminator, SV40 terminator, spy, yejM, SECG-LeuU, thrLABC, rrnB T1, hisLGDCBHAFI, metZWV, rrnC, xapR, aspA and arcA terminator.

### Usage Environment of Devices for Genetic Circuit

The devices for genetic circuit of the present invention can be used in various environment according to the desired final applications. The devices can be used, for example, but are not limited to, in a reactor vessel, in a living cell, in an artificial cell, in a cell ghost (i.e., red blood cell and platelet), in a dead cell, in an extracellular vesicle (e.g., exosome), in a virus, in an artificial virus or in an artificial virion and the like.

The devices for genetic circuit of the present invention can be introduced in the cell, for example, but are not limited to, by a method known per se utilizing micro injection, electroporation, lipofection, cell-penetrating peptide and the endocytotic character of DNA nano structure and the like.

The devices for genetic circuit of the present invention can be introduced in the body of animal and plant, for example, but are not limited to, by a, method known per se utilizing injection, oral administration, application to the skin/mucous membrane, suction, spray or diet and the like.

The devices for genetic circuit of the present invention can be used in various applications, such as, but are not limited to, in the biomarker detection, in gene therapy, in genetic modification (e.g., synthesis, addition/insertion, rewrite, editing, deletion and destruction), in inducing cell death, in inducing cell activation, in inducing cell inactivation, in preventive medicine, in regenerative medicine and/or in production of useful substance and the like. The devices for genetic circuit of the present invention can be formulated depend on the purpose, such as, but are not limited to, injection, oral administration, application to the skin/mucous membrane, suction, spray, diet or loading and the like. The devices for genetic circuit of the present invention can also be prepared for a reagent for detection and the like.

The devices for genetic circuit of the present invention can be used in the environment such as, but are not limited to, insect cell, insect, animal cell, animal, plant cell and/or plant and the like.

For insect cell, the following can be used, such as, but are not limited to, Spodoptera frugiperda cell (Sf cell), MG1 cell from Trichoplusia ni midgut, High Five™ cell from Trichoplusia ni egg, cell from Mamestra brassicae, cell from Estigmena acrea, Bombyx mori N cell (BmN cell), Sf9 cell (ATCC CRL1711) and/or Sf21 cell and the like.

For insect, the following can be used, such as, but are not limited to, silkworm larva, Drosophila and cricket.

For animal cell, the following can be used such as, but are not limited to, cell line (e.g., monkey COS-7 cell, monkey Vero cell, Chinese hamster ovary (CHO) cell, CHO/dhFr− cell, mouse L cell, mouse AtT-20 cell, mouse myeloma cell, rat GH3 cell and/or human FL cell), pluripotent stem cell (iPS and ES) from human and other mammals, primary culture cell from various tissue and the like. Additionally, zebrafish embryo, xenopus egg cell and the like can be also used.

For animal, the following can be used, such as, but are not limited to, experimental animals (e.g., rodent animals (e.g., mouse, rat and hamster) and rabbit), domesticated animals (e.g., pig, cow, goat, horse, sheep and mink), pets (e.g., dog and cat), primates (e.g., Homo sapiens, monkey, Rhesus macaque, marmoset, orangutan and chimpanzees) and fishes (e.g., zebrafish, killifish, goldfish, carp and tuna).

For plant cell, the following can be used, such as, but are not limited to, suspension cultured cell, callus, protoplast, leaf segment and root segment from various plant (for example, grain such as rice, wheat and corn; commercial crop such as tomato, cucumber, eggplant; garden plant such as carnation and Eustoma russellianum; experimental plant such as tobacco and Arabidopis thaliana) and the like.

The Kinds of the expression vector used for the catalyst (e.g., protein) preparation are not particularly limited, and include such as, plasmids derived from E. coli (e.g. pUB110, pTP5 and pC194), plasmids derived from yeast (e.g. pSH19 and pSH15), insect cell expression plasmids (e.g. pFast-Ba), animal cell expression plasmids (e.g. pA1-11, pXT1, pRc/CMV, pRc/RSV and pcDNAI/Neo), bacteriophage such as A phage, insect virus (e.g. vacuovirus) vector (e.g. BmNPV and AcNPV), animal virus (e.g. retrovirus, vaccinia virus, adenovirus and adeno-associated virus) vector and the like.

In addition, the vectors that express various tag fused proteins can be used, where tag includes such as, but are not limited to, FLAG tag peptide, HA tag peptide, MYC tag peptide, GFP tag peptide, MBP tag peptide, GST tag peptide, HIS tag, SNAP tag, ACP tag, CLIP tag, TAP tag and V5 tag and the like. Specifically, vector includes, for example, pSNAPf vector.

### Input (Activation Source)

The Activation source used in the present invention is not particularly limited, if the activation source can drive the change of the shape change element. For example, the activation source includes such as, but is not limited to, miRNA, siRNA, short hairpin RNA, dsRNA, lncRNA, ssDNA, dsDNA, nucleic acid nanostructure (e.g., DNA origami and RNA origami), IPTG, enzyme, protein, nucleic acid-protein complex, peptide, lipid, sugar chain, metabolite, ion, colloid, complex (coordination compound), light, pH, heat, electric field and magnetic field. Such activation sources can internally exist in the environment where the genetic circuit devices of the present invention are used, or can be applied externally.

### Output (Product)

The product outputted by the switch of the present invention is not particularly limited, and includes such as, protein (e.g., fluorescent protein, transcriptional repressor, transcription activator, enzyme, receptor protein and ligand protein), RNA (e.g., miRNA, siRNA, short hairpin RNA, riboswitch and lncRNA), DNA (e.g., ssDNA), ion, metabolite (e.g., ATP), nucleic acid nanostructure (e.g., DNA origami and RNA origami), light and heat and the like. The outputted product can be determined quantitatively through a method known per se. If several switches of present invention are used, the output (product) of one switch can function as an activation source (input) of other switches. In addition, the output product of the switches of the present invention can control the synthesis of materials (e.g., RNA, DNA and metabolite, etc.) in the environment where the genetic circuit devices of the present invention are used (e.g., cell). Furthermore, through the control of such materials (e.g., RNA, DNA and metabolite, etc.), the devices of the present invention can detect and/or prevent and/or treat the specific diseases. Moreover, through the control of such materials (e.g., RNA, DNA and metabolite, etc.), the devices of the present invention can increase and/or decrease the efficiency of production/decomposition of specific materials (e.g., biopharmaceuticals and hazardous substances) in the reactor vessel.

In the present invention, the anchor part on the base member is not particularly limited, if the anchor part can bind to any fixed part (e.g., the third fixed part) of the shape change element that exists in between the first- and second-fixing part of shape change element. The anchor part includes such as, but is not limited to, miRNA, siRNA, short hairpin RNA, dsRNA, lncRNA, ssDNA, dsDNA, nucleic acid nanostructure (e.g., DNA origami and RNA origami, etc.), IPTG, enzyme, protein (e.g., ligand protein and receptor protein, etc.), nucleic acid-protein complex, peptide, lipid, sugar chain, metabolite, ion, colloid and complex (coordination compound). Additionally, materials, which are used to fix the above described target gene, catalyst and shape change element of the present invention to the base member thereof, can be used.

### Activation Source for Binding

The activation source for binding of the present invention is not particularly limited, if the activation source for binding can induce the binding between any fixed part of the shape change element (e.g., the third fixed part that exists in between the first- and second-fixed part) and the anchor part of the base member (e.g., the third anchor part). The Activation source for binding includes such as, but is not limited to, miRNA, siRNA, short hairpin RNA, dsRNA, lncRNA, ssDNA, dsDNA, nucleic acid nanostructure (e.g., DNA origami and RNA origami), IPTG, enzyme, protein, nucleic acid-protein complex, peptide, lipid, sugar chain, metabolite, ion, colloid, complex (coordination compound), light, pH, heat, electric field and magnetic field. Such activation sources can internally exist in the environment where the genetic circuit devices of the present invention are used, or can be applied externally.

### Activation Source for Separation (Release)

The activation source for separation (release) is not particularly limited, if the activation source for separation (release) can induce the separation (release) of the binding between any fixed part of the shape change element (e.g., the third fixed part that exists in between the first- and second-fixed part) and the anchor part of the base member (e.g., the third anchor part). Activation source for separation (release) include such as, but is not limited to, miRNA, siRNA, short hairpin RNA, dsRNA, lncRNA, ssDNA, dsDNA, nucleic acid nanostructure (e.g., DNA origami and RNA origami), IPTG, enzyme, protein, nucleic acid-protein complex, peptide, lipid, sugar chain, metabolite, ion, colloid, complex (coordination compound), light, pH, heat, electric field and magnetic field. Such activation sources can internally exist in the environment where the genetic circuit devices of the present invention are used, or can be applied externally.

While the present invention is further specifically explained by the following Examples, the scope of the present invention is not limited to the Examples.

### EXAMPLES

### [Materials and Methods]

### 1. Plasmid Construction and Protein Purification

A DNA fragment containing a SNAPf-tag was amplified by PCR using the pSNAPf vector (NEB) as a template and cloned into the N-terminus of the T7 RNA polymerase sequence in pQE-30 (Qiagen) using In-Fusion HD (Clontech). The sfGFP and mCherry sequences were inserted into the Ndel-EcoRI site of pET32b (Merck). To improve protein production 31, the initial 21 codons of mCherry (MVSKGEEDNMAIIKEFMRFKV, SEQ ID No.73) were optimized to AT-rich ones (Original: ATG GTG AGC AAG GGC GAG GAG GAT AAC ATG GCC ATC ATC AAG GAG TTC ATG CGC TTC AAG GTG (SEQ ID No.71). After optimization: ATG GTT TCT AAA GGT GAA GAA GAT AAC ATG GCA ATT ATT AAA GAA TTT ATG CGT TTT AAA GTT (SEQ ID No.72). Quantification using [³⁵S]-Met showed that the modified mCherry sequence provided an approximately 4- to 5-fold increase in protein production in the PURE system (PUREfrex 2.0) compare to production with the original sequence and used in working example 6. The recombinant proteins were expressed as N-terminally His-tagged proteins in the E. coli BL21 strain. Typically, the cells were cultivated in 1 L of culture, sonicated, and centrifuged, and the proteins were purified with a publicly known method (Y. Shimizu et al., Cell-free translation reconstituted with purified components. Nat Biotechnol. 19, 751-755 (2001)) using a 5 mL HisTrap HP column (GE Healthcare). T7 RNAP_{SNAPf} was further purified with a 5 mL HiTrap Q column (GE Healthcare). The peak fractions were collected, buffer-exchanged to HT buffer (50 mM HEPES-KOH, pH 7.6, 100 mM KCl, 10 mM MgCl2, 40% glycerol, and 7 mM b-ME), frozen in liquid N2 and stored at −80° C.

### 2. Scaffolds and Nucleotides

Single-stranded M13mp18 DNA was purchased from NEB and used as the scaffold for DNA origami. Unmodified staple strands were purchased from Sigma-Genosys as Oligonucleotide Purification Cartridge (OPC) grade. Fluorescent dye (Cy3 and Cy5) modified staples were purchased from Sigma-Genosys as PAGE-purified. Dual-biotin modified staples were purchased from IDT as HPLC-purified. Amino-modified staples were purchased from IDT as Dual-HPLC-purified. The SNAP-ligand was covalently attached to the amino-modified staples by mixing the staples and BG-GLA-NHS (NEB, dissolved in DMSO) with a publicly known method (N. D. Derr et al., Tug-of-war in motor protein ensembles revealed with a programmable DNA origami scaffold. Science 338, 662-665 (2012)). The label efficiency of the SNAP-ligand was estimated to be 95-98% by a gel-shift assay using purified SNAP-tag protein. Photoreactive cross-linking nucleotide (3-cyanovinylcarbazole, cnvK) modified staples were purchased from Tsukuba Oligo Service as HPLC-purified. Followings are the primer sequences used in this patent specification.

TABLE 1
DNA oligonucleotide sequences of the handles used for anchoring.
The positions are the same as those in ref 16 (Wickham S.F.J. et al. Nat. Nanotechnol. 6.
166-169 (2011). Tile type B (10.4 bp t SL. SNAP tag ligand. dB. dual biotin.
B. biotin. x. cnvK.

| FIG. # | Fixing Target | Sequence | Position | Seq. # |
|---|---|---|---|---|
| 1 | T7 | SL-tttttttGCATTAATCTCTAGAGGATCCCCAGTTGGGT | M-7t20e | 1 |
| 2-4 | T7 | SL-tttttttAGCTAAATTTGATTCCCAATTCTCTGAATAT | M-7t12e | 2 |
| 2A-D | Gene (4 nm) | dB-tttttttTATAACAGCGGTTGTACCAAAAAGCCTTTAT | M-7t12f | 3 |
| 2A-D | Gene (24 nm) | dB-tttttttAACTGACCGTAATGCCACTACGACGAGGGTA | M-7t4e | 4 |
| 2A-D | Gene (32 nm) | dB-tttttttCGCGACCTCATCTTTGACCCCCAGGCAGGGAG | M-3t4e | 5 |
| 2A-D | Gene (50 nm) | dB-tttttttCGCCACCAAGGAGGTTGAGGCCAGAAACATGA | M-1t4e | 6 |
| 2A-D | Gene (70 nm) | dB-tttttttACCGCCTCGCCAGAATGGAAAGCTGAGTAAC | M-5t4e | 7 |
| 2E | mCherry (50 nm) | dB-tttttttCGCCACCAAGGAGGTTGAGGCAGAAACATGA | M-1t4e | 8 |
| 2E | sfGFP (24 nm) | dB-tttttttGCATTAATCTCTAGAGGATCCCCAGTTGGGT | M-7t20e | 68 |
| 2E | sfGFP (32 nm) | dB-tttttttACTCACATTGAAATTGTTATCCGCGGTGCGG | M-3t20e | 9 |
| 2E | sfGFP (50 nm) | dB-tttttttGAATTATCTCATCAATATAATCCTTTCAATT | M-1t20e | 10 |
| 2E | sfGFP (70 nm) | dB-tttttttTTAATTTTCATATCAAAATTATTTAACGGAT | M-5t20e | 11 |
| 3 | sfGFP (50 nm) | dB-tttttttCGCCACCAAGGAGGTTGAGGCAGAAACATGA | M-1t4e | 6 |
| 4 | Sensor (50 nm) | dB-tttttttCGCCACCAAGGAGGTTGAGGCAGAAACATGA | M-1t4e | 6 |
| 4 | Sensor (70 nm) | dB-tttttttACCGCCTCGCCAGAATGGAAAGCTGAGTAAC | M-5t4e | 7 |
| 4 | Gene (70 nm) | B- | M5t4e | 12 |
| | | tttttTGCAxGCGTtttttttttttttttttttttttttttt | | |
| | | ttACGCGTGCAtttttACCGCCTCGCCAGAATGGAAAGCTGA | | |
| | | GTAAC | | |
| 1-4 | Purification1 | GGAACTTCAGCCCAACTAACATTTT_ACCGCCACTTTTAT | M9t2f | 13 |
| | | GATACAGGAGTGTATCATACAT | | |
| 1-4 | Purification2 | GGAACTTCAGCCCAACTAACATTTT_GGTTTAACTTTTAT | M9t20f | 14 |
| | | TAGACTTTACAAACTTGAGGAT | | |
| 15 | mCherry(sfCherry) | dB-tttttttCGCCACCAAGGAGGTTGAGGCAGAAACATGA | M1t4e | 8 |
| | (50 nm) | | | |
| 15 | sfGFP (50 nm) | dB-tttttttGAATTATCTCATCAATATAATCCTTTCAATT | M1t20e | 15 |

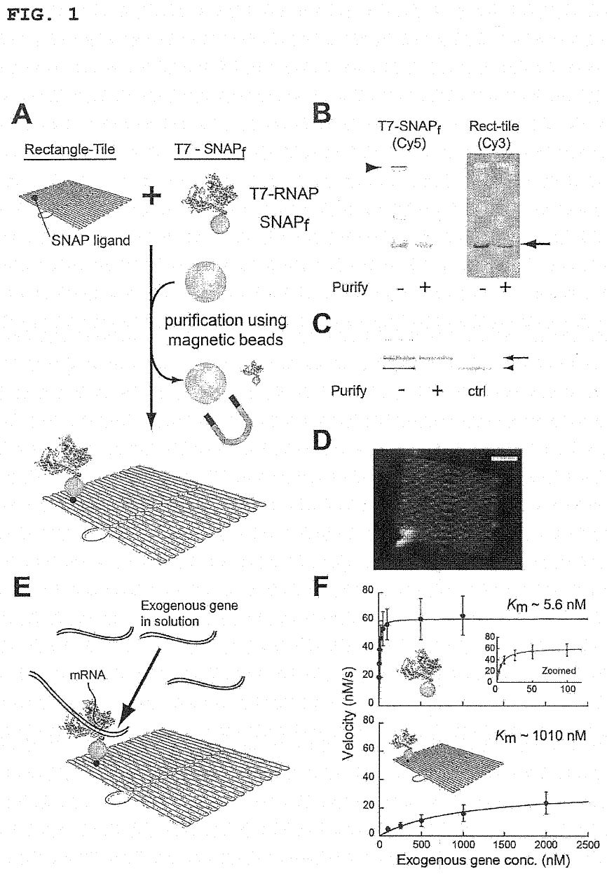

TABLE 2
PCR primers for FIG. 1-3.
Linker length: length from the attachment point to the promoter sequence.

| | Forward | Fixing | | |
|---|---|---|---|---|
| FIG. # | or Reverse, name of | direction, Linker | Sequence | SEQ # |
| 2B-C | F.p32a130 | 5′, 44 nm | dB-GTGGCGAGCCCGATC | 16 |
| 2B-C | R.T7 terminator | 5′, 44 nm | GCTAGTTATTGCTCAGCGG | 17 |
| 2B | F.p32a65 | 3′, 1089 bp | GCCGGTGATGCCG | 18 |
| 2B | R.p32aR130 | 3′, 1089 bp | dB-CTGCGCGTAACCACCA | 19 |
| 2D | F.32a65 | 5′, 22 nm | dB-GCCGGTGATGCCG | 20 |
| 2D | F.p32a130 | 5′, 44 nm | dB-GTGGCGAGCCCGATC | 16 |
| 2D | F.p32a195 | 5′, 66 nm | dB-AACAGTCCCCCGGCCAC | 21 |
| 2D | R. T7 terminator | 5′, 22-66 nm | GCTAGTTATTGCTCAGCGG | 17 |
| 2E | F.p32a130 | 5′, 44 nm | dB-GTGGCGAGCCCGATC | 16 |
| 2E | R. T7 terminator | 5′, 44 nm | GCTAGTTATTGCTCAGCGG | 17 |
| 3 | F.p32a130, for | 5′, 44 nm | dB-GTGGCGAGCCGATC | 16 |
| | nano-chip | | | |
| 3 | F.p32a130, for DNA- | Diffusing, 44 nm | GTGGCGAGCCCGATC | 22 |
| | initiated | from 5′ end | | |
| 3 | F.p32a130, for mRNA- | Diffusing, 44 nm | GTGGCGAGCCCGATC | 22 |
| | initiated template | from 5′ end | | |
| 3 | R. T7 terminator | 5′, 44 nm | GCTAGTTATTGCTCAGCGG | 17 |

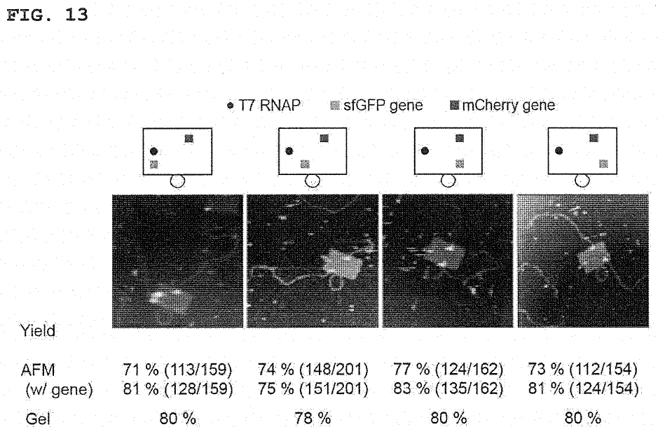

TABLE 3
PCR primers for FIG. 4 and 17-30
The template DNA was amplified by PCR with following primers. Typically, two steps PCR was used for Supplementary
FIGS. 13-24, in which the universal sequence was added by first PCR and sensor sequence was added through
second PCR. We note that PCR products of 22 nm dsDNA linker length were amplified directly from plasmid by
single step PCR. Primers used in the first or second PCR are designated as * or **, respectively, dB: dual biotin.
For ON switch sensor, we named the primers using H′ and effective loop length L′ (= T + L). For example, H12L31
has 12 nt of H′ and 31 nt of L′ (H = H′ - 1 = 12 - 1 = 11 nt, T = T + H)- H = 21 - 11 = 10 nt (Note: let-7 is
21 nt), L = L′ - T = 31 - 10 = 21 nt).
For easy handling, we renamed the miRNA in the primer as followings: hsa-miR-1-3p to ″a″, hsa-miR-92a-3p to ″c″,
hsa-miR-197-3p to ″d″, hsa-miR-224-5p to ″e″, hsa-miR-365-3p to ″f″ and hsa-miR-183-5p to ″g″.
***If a sequence includes ″iSp9″ (triethylene glycol), ″Seq. #″ indicates a sequence upstream therof.

| | Forward or Reverse, | Fixing direction, | | Seq. | PCR | |
|---|---|---|---|---|---|---|
| FIG. # | name of primer | dsDNA Linker length | Sequence (5′-3′) | #*** | step | Note |
| 17 | F.p32b_no65_25 | -, 8.5 nm | GCCGGTGATGCCGatcccgcgaaattaatacgactcactataggg | 23 | * | add universal sequence at 25 bp |
| | | | | | | upstream of T7 promoter |
| 17 | F.p32b_no65_45 | -, 15 nm | GCCGGTGATGCCCgaggatcgagatcgatctcgatccc | 24 | * | add universal sequence at 45 bp |
| | | | | | | upstream of T7 promoter |
| 17 | F.p32b_no65_85 | -, 29 nm | GCCGGTGATGCCGccagcaaccgcacctgtggcgccacgatgc | 25 | * | add universal sequence at 55 bp |
| | | | gtccggc | | | upstream of T7 promoter |
| 17 | F.0T_p32b65_dB | 5′, 8.5, 15, 22 | dB-iSp9-GCCGGTGATGCCG | — | ** | dB + polyT + universal sequence |
| | | and 29 nm | | | | |
| 17 | F.5T_p32b65_dB | 5′, 8.5, 15, 22 | dB-ttttt-iSp9-GCCGGTGATGCCG | — | ** | dB + polyT + universal sequence |
| | | and 29 nm | | | | |
| 17 | F.10T_p32b65_dB | 5′, 8.5, 15, 22 | dB-tttttttttt-iSp9-GCCGGTGATGCCG | 26 | ** | dB + polyT + universal sequence |
| | | and 29 nm | | | | |
| 17 | F.15T_p32b65_dB | 5′, 8.5, 15, 22 | dB-ttttttttttttttt-iSp9-GCCGGTGATGCCG | 27 | ** | dB + polyT + universal sequence |
| | | and 29 nm | | | | |
| 17 | F.20T_p32b65_dB | 5′, 8.5, 15, 22 | dB-tttttttttttttttttttt-iSp9-GCCGGTGATGCCG | 28 | ** | dB + polyT + universal sequence |
| | | and 29 nm | | | | |
| 17 | F.30T_p32b65_dB | 5′, 8.5, 15, 22 | dB-tttttttttttttttttttttttttttttt-iSp9- | 29 | ** | dB + polyT + universal sequence |
| | | and 29 nm | GCCGGTGATGCCG | | | |
| 17 | F.40T_p32b65_dB | 5′, 8.5, 15, 22 | dB-tttttttttttttttttttttttttttttttttttttttt- | 30 | ** | dB + polyT + universal sequence |
| | | and 29 nm | iSp9-GCCGGTGATGCCG | | | |
| 17 | F.50T_p32b65_dB | 5′, 8.5, 15, 22 | dB-ttttttttttttttttttttttttttttttttttttttttt | 31 | ** | dB + polyT + universal sequence |
| | | and 29 nm | ttttttttt-iSp9-GCCGGTGATGCCG | | | |
| 17 | F.60T_p32b65_dB | 5′, 8.5, 15, 22 | dB-ttttttttttttttttttttttttttttttttttttttttt | 32 | ** | dB + polyT + universal sequence |
| | | and 29 nm | ttttttttttttttttttt-iSp9-GCCGGTGATGCCG | | | |
| 17 | R. T7 promoter | 5′, 8.5, 15, 22 | GCTAGTTATTGCTCAGCGG | 17 | *, ** | T7 terminator |
| | | and 29 nm | | | | |
| 18 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 18 | F.H15L25_p32b65_dB | 5′, 15 nm | dB-tcttcactatacaacctactacctcatttttttttttttt | 33 | ** | let-7 ON-switch H15L25 + |
| | | | ttttgtaggttgtatagtg-iSp9-GCCGGTGATGCCG | | | universal sequence |
| 18 | F.H14L27_p32b65_dB | 5′, 15 nm | dB-tcttcactatacaacctactacctcattttttttttttttt | 34 | ** | let-7 ON-switch H14L27 + |
| | | | tttttaggttgatagtg-iSp9-GCCGGTGATGCCG | | | universal sequence |
| 18 | F.H13L29_p32b65_dB | 5′, 15 nm | dB-tccttcactatacaacctactacctcatttttttttttttt | 35 | ** | let-7 ON-switch H13L29 + |
| | | | tttttAaggttgtatagtg-iSp9-GCCGGTGATGCCG | | | universal sequence |
| 18 | F.H12L31_p32b65_dB | 5′, 15 nm | dB-tcttcactatcaacctactacctcatttttttttttttttt | 36 | ** | let-7 ON-switch H12L31 + |
| | | | tttttggttgtatagtg-iSp9-GCCGGTGATGCCG | | | universal sequence |
| 18 | F.H11L33_p32b65_dB | 5′, 15 nm | dB-tcttcactatacaacctactacctcattttttttttttttt | 37 | ** | let-7 ON-switch H11L33 + |
| | | | tttttttgttgtatagtg-iSp9-GCCGGTGATGCCG | | | universal sequence |
| 18 | F.H10L35_p32b65_dB | 5′, 15 nm | dB-tcttcactatacaacctactacctcattttttttttttttt | 38 | ** | let-7 ON-switch H10L35 + |
| | | | ttttttttttgtatagtg-iSp9-GCCGGTGATGCCG | | | universal sequence |
| 18 | F.H9L37_p32b65_dB | 5′, 15 nm | dB-tcttcactatacaacctactacctcattttttttttttttt | 39 | ** | let-7 ON-switch H9L37 + |
| | | | ttttttttAtgtatagtg-iSp9-GCCGGTGATGCCG | | | universal sequence |

TABLE 4

| | | | | | | |
|---|---|---|---|---|---|---|
| 18 | F.H8L39_p32b65_dB | 5′, 15 nm | dB-tcttcactatacaacctactacctcattttttttttttttt | 40 | ** | let-7 ON-switch H8L39 + |
| | | | ttttttttAAgtatagtg-iSp9-GCCGGTGATGCCG | | | universal sequence |
| 18 | F.H7L41_p32b65_dB | 5′, 15 nm | dB-tcttcactatacaacctactacctcattttttttttttttt | 41 | ** | let-7 ON-switch H7L41 + |
| | | | ttttttttttttatagtg*iSp9-GCCGGTGATGCCG | | | universal sequence |
| 18 | F.H6L43_p32b65_dB | 5′, 15 nm | dB-tcttcactatacaacctactacctcattttttttttttttt | 42 | ** | let-7 ON-switch H6L43 + |
| | | | ttttttttttttAatagtg-iSp9-GCCGGTGATGCCG | | | universal sequence |
| 18 | F.H5L45_p32b65_dB | 5′, 15 nm | dB-tcttcactatacaacctactacctcattttttttttttttt | 43 | ** | let-7 ON-switch H5L45 + |
| | | | ttttttttttttttagtg-iSp9-GCCGGTGATGCCG | | | universal sequence |
| 18 | R. T7 terminator | 5′, 15 nm | GCTAGTTATTGCTCAGCGG | 17 | *, ** | T7 terminator |
| 19 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 19 | F.H12L31_p32b65_ | 5′, 15 nm | dB-tctGcactatacaacctactacctcattttttttttttttt | 44 | ** | let-7 ON-switch + universal |
| | dB_GC | | tttttggttgtatagtgC-iSp9-GCCGGTGATGCCG | | | sequence (support hybridization |
| | | | | | | 2 bp) |
| 19 | F.H12L31_p32b65_ | 5′, 15 nm | dB-tcCGcactatacaacctactacctcattttttttttttttt | 45 | ** | let-7 ON-switch + universal |
| | dB_CGC | | ttttggttgtatagtgCG-iSp9-GCCGGTGATGCCG | | | sequence (support hybridization |
| | | | | | | 3 bp) |
| 19 | F.H12L31_p32b65_ | 5′, 15 nm | dB-tcCGcactatacaacctactacctcattttttttttttttt | 46 | ** | let-7 ON-switch + universal |
| | dB_CCGC | | tttggttgtatagtgCGG-iSp9-GCCGGTGATGCCG | | | sequence (support hybridization |
| | | | | | | 4 bp) |
| 19 | R. T7 terminator | 5′, 15 nm | GCTAGTTATTGCTCAGCGG | 17 | *, ** | T7 terminator |
| 20 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 20 | F.H12L31_p32b65_ | 5′, 15 nm | dB-tcttcactatacaacctactacctcattttttttttttttt | 36 | ** | let-7 ON-switch H12L31 + |
| | dB | | ttttttggttgtatagtg-iSp9-GCCGGTGATGCCG | | | universal sequence |
| 20 | R. T7 terminator | 5′, 15 nm | GCTAGTTATTGCTCAGCGG | 17 | *, ** | T7 terminator |
| 21 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 21 | F.H12L31_p32b65_ | 5′, 15 nm | dB-tcttcactatacaacctactacctcattttttttttttttt | 36 | ** | let-7 ON-switch H12L31 + |
| | dB | | ttttttggttgtatagtg-iSp9-GCCGGTGATGCCG | | | universal sequence |
| 21 | F.miRa-H14L27_ | 5′, 15 nm | dB-tcttcatacatacttctttacattccatttttttttttttt | 47 | ** | miRa(miR-1-3p) ON-switch + |
| | p32b65_dB | | ttttaagaagtatgtatg-iSp9-GCCGGTGATGCCG | | | universal sequence |
| 21 | F.miRb-H11L33_ | 5′, 15 nm | dB-tcttcccacacacttccttacattccatttttttttttttt | 48 | ** | miRb(miR-206) ON-switch + |
| | p32b65_dB | | tttttttaagtgtgtggg-iSp9-GCCGGTGATGCCG | | | universal sequence |
| 21 | F.miRc-H9L37_ | 5′, 15 nm | dB-tcttcacaggccgggacaagtgcaatatttttttttttttt | 49 | ** | miRc(miR-92a-3p) ON-switch + |
| | p32b65_dB | | tttttttttcggcctgtg-iSp9-GCCGGTGATGCCG | | | universal sequence |
| 21 | F.miRd-H9L37_ | 5′, 15 nm | dB-tcttcgctgggtggagaaggtggtgaatttttttttttttt | 50 | ** | miRd(miR-197-3p) ON-switch + |
| | p32b65_dB | | tttttttttcacccagcg-iSp9-GCCGGTGATGCCG | | | universal sequence |
| 21 | F.miRe-H10L35_ | 5′, 15 nm | dB-tcttcaacggaaccactagtgacttgttttttttttttttt | 51 | ** | miRe(miR-224-5p) ON-switch + |
| | p32b65_dB | | tttttttAggttccgttg-iSp9-GCCGGTGATGCCG | | | universal sequence |
| 21 | F.miRf-H13L29_ | 5′, 15 nm | dB-tcttcataaggatttttaggggcattatttttttttttttt | 52 | ** | miRf(miR-365a-3p) ON-switch + |
| | p32b65_dB | | ttttAaaaaatccttatg-iSp9-GCCGGTGATGCCG | | | universal sequence |
| 21 | F.miRg-H13L29_ | 5′, 15 nm | dB-tcttcagtgaattctaccagtgccatatttttttttttttt | 53 | ** | miRg(miR-183-5p) ON-switch + |
| | p32b65_dB | | tttttgtagaattcactg-iSp9-GCCGGTGATGCCG | | | universal sequence |
| 21 | R. T7 terminator | 5′, 15 nm | GCTAGTTATTGCTCAGCGG | 17 | *, ** | T7 terminator |

TABLE 5

| | | | | | | |
|---|---|---|---|---|---|---|
| 22 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 22 | F.OFF_a_661_ | 5′, 15 nm | dB-actatacaacctttttttttttttttttttttttttttttt | 54 | ** | let-7 OFF-switch + universal |
| | p32b65_dB | | tttttttttttttttttttttttttttttttttttttactacct | | | sequence (support hybridization |
| | | | ca-iSp9-GCCGGTGATGCCG | | | 0 bp) |
| 22 | F.OFF_h4_ | 5′, 15 nm | dB-ActatacaaccCGcctttttttttttttttttttttttttt | 55 | ** | let-7 OFF-switch + universal |
| | p32b65_dB | | ttttttttttttttttttttttggCGtactacctca-iSp9- | | | sequence (support hybridization |
| | | | GCCGGTGATGCCG | | | 4 bp) |
| 22 | F.OFF_h6_ | 5′, 15 nm | dB-ActatacaaccCCGCCCtttttttttttttttttttttttt | 56 | ** | let-7 OFF-switch + universal |
| | p32b65_dB | | tttttttttttttttttttttacctca-iSp9- | | | sequence (support hybridization |
| | | | GCCGGTGATGCCG | | | 6 bp) |
| 22 | F.OFF_h7_ | 5′, 15 nm | dB-ActatacaaccCCGCCCCttttttttttttttttttttttt | 57 | ** | let-7 OFF-switch + universal |
| | p32b65_dB | | tttttttttttttttttttGGGGCGGtactacctca-iSp9- | | | sequence (support hybridization |
| | | | GCCGGTGATGCCG | | | 7 bp) |
| 22 | F.OFF_h10_ | 5′, 15 nm | dB-ActatacaaccCCCGTCCCCCtttttttttttttttttttt | 58 | ** | let-7 OFF-switch + universal |
| | p32b65_dB | | ttttttttttttttttGGGGGACGGGtactacctca-iSp9- | | | sequence (support hybridization |
| | | | GCCGGTGATGCCG | | | 10 bp) |
| 22 | R. T7 terminator | 5′, 15 nm | GCTAGTTATTGCTCAGCGG | 17 | *, ** | T7 terminator |
| 23 | F.p32b_no65_19 | -, 6.5 nm | GCCGGTGATGCCGcgaaattaatacgactcactataggggaatt | 59 | * | add universal sequence at 19 bp |
| | | | gtg | | | upstream of T7 promoter |
| 23 | F.AND_let7-197_ | 5′, 6.5 nm | dB-tcactatacaacctactacctcatttttttttttggttgta | 60 | ** | let-7/miRd(let-7/miR-197-3p) 2- |
| | p32b65_dB_v2b | | tagtgtgctgggtggagaaggtggtgaattttttttaccaccca | | | input AND-switch + universal |
| | | | gc-iSp9-GCCGGTGATGCCG | | | sequence |
| 23 | R. T7 terminator | 5′, 6.46 nm | GCTAGTTATTGCTCAGCGG | 17 | *, ** | T7 terminator |
| 24 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 24 | F.3And_let7bc206- | 5′, 15.3 nm | dB-tcactatacaacctactactcatttttttttttggttgtat | 61 | ** | let-7/miRb/miRc(let-7/miR-206/ |
| | 92a_p32b65_dB | | agtgtcccacacacttccttacattccatttttttttttaagtg | | | miR-92a-3p) 3-input AND-switch + |
| | | | tgtgggtcacaggccgggacaagtgcaatattttttttcggcct | | | universal sequence |
| | | | gtg-iSp9-GCCGGTGATGCCG | | | |
| 24 | 3And_let7bd_206- | 5′, 15.3 nm | dB-tcactatacaacctactacctccatttttttttttggttgt | 62 | ** | let-7/miRb/miRd(let-7/miR-206/ |
| | 197_p32b65-dB | | atagtgtcccacacacttccttacattccatttttttttttaag | | | miR-197-3p) 3-input AND-switch + |
| | | | tgtgtgggtgctgggtggagaaggtggtgaattttttttaccac | | | universal sequence |
| | | | ccagc-iSp9-GCCGGTGATGCCG | | | |
| 24 | 3And_let7fg_365a- | 5′, 15.3 nm | dB-tcactatacaacctactacctcatttttttttttggttgtt | 63 | ** | let-7/miRf/miRg(let-7/miR-365a- |
| | 183_p32b65-dB | | agtgtcataaggatttttaggggcattattatttaaaaaatcct | | | 3p/miR-183-5p) 3-input AND- |
| | | | tatgtcagtgaattctaccagtgccatattttttgtagaattca | | | switch + universal sequence |
| | | | ctg-iSp9-GCCGGTGATGCCG | | | |
| 24 | R. T7 promoter | 5′, 15.3 nm | GCTAGTTATTGCTCAGCGG | 17 | *, ** | T7 terminator |
| 25 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 25 | F.p32b_no65_85 | -, 29 nm | GCCGGTGATGCCGccagcaaccgcacctgtggcgccacgatgcg | 25 | * | add universal sequence at 55 bp |
| | | | tccggc | | | upstream of T7 promoter |
| 25 | F.3And_letbc_206- | 5′, 15 nm, 29 nm | dB-tcactatacaacctactacctcatttttttttttggttgta | 64 | ** | let-7/miRb/miRc(let-7/miR-206/ |
| | 92_p32b65_dB | | tagtgtcccacacacttccttacattccatttttttttttaagt | | | miR-92a-3p) 3-input AND- |
| | | | gtgtgggtcacaggccgggacaagtgcaatattttttttcggcc | | | switch + universal sequence |
| | | | tgtg-iSp9-GCCGGTGATGCCG | | | |
| 25 | R. T7 promoter | 5′, 15 nm, 29 nm | GCTAGTTATTGCTCAGCGG | 17 | *, ** | T7 terminator |

TABLE 6

| | | | | | | |
|---|---|---|---|---|---|---|
| 26 | p.ET32-5′ 130 DB | 5′, 44 nm | dB-gtggcgagcccgatctt | 65 | * | add dual-biotin at 130 bp |
| 26 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 26 | F.H12L31_p32b65_ | 5′, 15 nm | dB-tcttcactatacaacctactacctcattttttttttttttt | 36 | ** | let-7 ON-switch H12L31 + |
| | dB | | ttttttggttgtatagtg-iSp9-GCCGGTGATGCCG | | | universal sequence |
| 27 | R. T7 promoter | 5′, 15 nm, 44 nm | GCTAGTTATTGCTCAGCGG | 17 | *, ** | T7 terminator |
| 27A-B | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 27A-B | F.miRd-H9L37_ | 5′, 15 nm for | dB-tcttcgctgggtggagaaggtggtgaatttttttttttttt | 50 | ** | miRd(miR-197-3p) ON-switch + |
| | p32b65_dB | Chip1 | tttttttttcacccagcg-iSp9-GCCGGTGATGCCG | | | universal sequence |
| 27A-B | F.H12L31_p32b65_ | 5′, 15 nm for | dB-tcttcactatacaacctactacctcattttttttttttttt | 36 | ** | let-7 ON-switch H12L31 + |
| | dB | Chip2 | ttttttggttgtatagtg-iSp9-GCCGGTGATGCCG | | | universal sequence |
| 27C-E | F.p32b_no65_19 | -, 6.5 nm | GCCGGTGATGCCGcgaaattaatacgactcactataggggaatt | 59 | * | add universal sequence at 19 bp |
| | | | gtg | | | upstream of T7 promoter |
| 27C-E | F.And_bc_206-92a_ | 5′, 6.5 nm for | dB-tcccacacacttccttacattccatttttttttttaagtgt | 66 | ** | miRb/miRc(miR-206/miR-92a-3p) |
| | p32b65_dB | Chip1 | gtgggtcacaggccggacaagtgcaatatttttttt-iSp9- | | | 2-input AND-switch + universal |
| | | | GCCGGTGATGCCG | | | sequence |
| 27C-E | F.AND_let7-197_ | 5′, 6.5 nm for | dB-tcactatacaacctactacctcatttttttttttggttgta | 60 | ** | let-7/miRd(let-7/miR-197-3p) 2- |
| | p32b65_dB_v2b | Chip2 | tagtgtgctgggtggagaaggtggtgaattttttttaccaccca | | | input AND-switch + universal |
| | | | gc-iSp9-GCCGGTGATGCCG | | | sequence |
| 27 | R. T7p_let7_R_ | 5′, 15 nm, 6.5 nm | A[2OMe]TATACAACCTACTACCTCACCCCTATAGTGAGT | 67 | ** | for let-7 production (T7promoter |
| | gg_2OMe | for Chip1 | CGTATTAATTTCGCGG | | | + gg + 2OMe |
| 27 | R. T7 promoter | 5′, 15 nm, 6.5 nm | GCTAGTTATTGCTCAGCGG | 17 | *, ** | T7 terminator |
| | | for Chip2 | | | | |
| 28 | F.p32b_no65_19 | -, 6.5 nm | GCCGGTGATGCCGcgaaattaatacgactcactataggggaatt | 59 | * | add universal sequence at 19 bp |
| | | | gtg | | | upstream of T7 promoter |
| 28 | F.And_bc_206-92a_ | 5′, 6.5 nm for | dB-tcccacacacttccttacattccatttttttttttaagtgt | 66 | ** | miRb/miRc(miR-206/miR-92a-3p) |
| | p32b65_dB | Chip1 | gtgggtcacaggccggacaagtgcaatatttttttt-iSp9- | | | 2-input AND-switch + universal |
| | | | GCCGGTGATGCCG | | | sequence |
| 28 | R. T7p_let7_R_gg_ | 5′, 6.5 nm for | A[2OMe]TATACAACCTACTACCTCACCCCTATAGTGAGT | 67 | ** | for let-7 production (T7promoter |
| | 2OMe | Chip2 | CGTATTAATTTCGCGG | | | + gg + 2OMe |
| 28 | F.AND_let7-197_ | 5′, 6.5 nm for | dB-tcactatacaacctactacctcatttttttttttggttgta | 60 | ** | let-7/miRd(let-7/miR-197-3p) 2- |
| | p32b65_dB_v2b | Chip2 | tagtgtgctgggtggagaaggtggtgaattttttttaccaccca | | | input AND-switch + universal |
| | | | gc-iSp9-GCCGGTGATGCCG | | | sequence |
| 28 | R. T7 promoter | 5′, 6.5 nm for | GCTAGTTATTGCTCAGCGG | 17 | *, ** | T7 terminator |
| | | Chip2 | | | | |
| 29 | F.T7_Fp32a195-dB | 5′, 66 nm | dB-aacagtcccccggccac | 21 | * | add dual-biotin at 195 bp |
| | | | | | | upstream of T77 promoter |
| 29 | R. T7 terminator | 5′, 66 nm | GCTAGTTATTGCTCAGCGG | 17 | * | T7 terminator |
| 30 | F.32b_no65_45 | -, 15 nm | GCCGGTGATGCCGgaggatcgagatcgatctcgatccc | 24 | * | add universal sequence at 45 bp |
| | | | | | | upstream of T7 promoter |
| 30 | F.3And_let7bc_206- | 5′, 15 nm, 29 nm | dB-tcactatacaacctactacctcatttttttttttggttgta | 64 | ** | let-7/miRb/miRc(let-7/miR-206/ |
| | 92a_p32b65_dB | | tagtgtcccacacacttccttacattccatttttttttttaagt | | | miR-92a-3p) 3-input AND- |
| | | | gtgtgggtcacaggccgggacaagtgcaatattttttttcggcc | | | switch + universal sequence |
| | | | tgtg-iSp9-GCCGGTGATGCCG | | | |
| 30 | R. T7 terminator | 5′, 15 nm, 29 nm | GCTAGTTATTGCTCAGCGG | 17 | * | T7 terminator |

TABLE 7

| | | | | | | |
|---|---|---|---|---|---|---|
| 33 | F.p32b115_dB | 5′, 39 nm | dB-TCCCCATCGGTGATG | 69 | * | without sensor sequence |
| 33 | R. T7 terminator | 5′, 39 nm | GCTAGTTATTGCTCAGCGG | 17 | * | T7 terminator |
| 33 | F.p32b115_dB | 5′, 39 nm for | dB-TCCCCATCGGTGATG | 69 | * | without sensor sequence |
| | | Chip1 | | | | |
| 34 | R. T7p_miR-197- | 5′, 39 nm for | G[2OMeC]TGGGTGGAGAAGGTGGTGAACCC | 70 | * | for miRb(miR-205) production |
| | 39_R_gg_2OMe | Chip1 | CTATAGTGAGTCGTATTAATTTCGCGG | | | (T7promoter + gg + 2′OMe |
| | | | | | | modification) |
| 34 | F.p32b_no65_45 | -, 15 nm | GCCGGTGATGCCGgaggatcgagatcgatctcgatccc | 24 | * | add universal sequence at 45 bp |
| | | | | | | upstream of T7 promoter |
| 34 | F.3And_let7bc_206- | 5′, 15 nm for | dB-tcactatacaacctactacctcatttttttttttggttgta | 64 | ** | let-7/miRb/miRc(let-7/miR-206/ |
| | 92a_p32b65_B | Chipp2 | tagtgtcccacacacttccttacattccatttttttttttaagt | | | miR-92a-3p) 3-input AND- |
| | | | gtgtgggtcacaggccgggacaagtgcaatattttttttcggcc | | | switch + universal sequence |
| | | | tgtg-iSp9-GCCGGTGATGCCG | | | |
| 34 | R. T7 terminator | 5′, 15 nm for | GCTAGTTATTGCTCAGCGG | 17 | * | T7 terminator |
| | | Chip2 | | | | |

TABLE 8

| name | sequence (5′ to 3′) | SEQ # |
|---|---|---|
| complementary for PacI | TAAGGGAAAATTAATTAATAGCGACGAT | 74 |
| complementary for BglII | TAGCCTTTGTAGATCTCTCAAAAATA | 75 |
| p8064_F4475_20 nt | TAATAGCGACGATTTACAGA | 76 |
| p8064_R24_23 nt | TACAAAGGCTATCAGGTCATTGC | 77 |
| 7X7 NC 19 new | AAAGAATACAGCGGGGTCATTGCTGGAGGTG | 78 |
| 7X7 NC 21 new | TGGTGTGAATCCGCCGGGCGCGGGTGGTGCC | 79 |
| 7X7 NC 23 new for cnvK | GTTTCTTTGTCACTGTTGCCCTGCCAACCGCA | 80 |
| 7X7 NC 25 new | TCCAGCATTTTTTGGGCTAGGGCG | 81 |
| 7X7 NC 26 new | ATCCCACGCAACCAGCATCCTCA | 82 |
| 7X7 NC 27 new for cnvk | AGAATGCCAACGGCAGAACGTCAG | 83 |
| 7X7 NC 34 new | CTGGCAACCCGCCGCGCTTAATGGTTGTGTA | 84 |
| 7X7 NC 35 new | TAACGGAACCTCCGGCCAGAGCAAAAAAAGC | 85 |
| 7X7 NC 36 new for cnvK | CGTGGTGCGCGGTCCGTTTTTTCAGGGT | 86 |
| 7X7 NC 44 (snap) | CATCGACCGCCAGCAGTTGGGCGCCTGAGTA | 87 |
| 7X7 NC 45 (bio) | CGCACAGGGCGGATCAAACTTAAGGCGAAAC | 88 |
| 7X7 NC 44 (bio) | GAAGAACACAATATTACCGCCAG | 89 |
| 7X7 NC 45 new | GTACAGCGTCACCGGAAACAATC | 90 |
| 46 new for cnvK | GTGAGAGCAGAGGTGGAGCCGCC | 91 |
| 7X7 NC 54 new | CCATTGCATCGCTATTACGCCAGATCGGTGC | 92 |
| 7X7 NC 55 new | GCTGGTAAGTGCTGCAAGGCGATTCATTCAGG | 93 |
| 56 new for cnvK | ACGGGAACGGGTTTTCCCAGTCATGGTGCCG | 94 |
| 7X7 NC 48 tag | GGAACTTCAGCCCAACTAACATTTTgtcCATCACGCATCAATCGTC | 95 |
| 7X7 NC 85 tag | GGAACTTCAGCCCAACTAACATTTTtttgCCCGAACGCGTATTAAGCAAATGA | 96 |

TABLE 9

| name | sequence (5′ to 3′) | SEQ # |
|---|---|---|
| Zebra non-template | Biotin-TGCAxGCGTgatcgatctcgatcccgcgaaat taatacgactcactatagg gat | 97 |
| Zebra template | GCTCCATAGCTTTAAATCGATGGGATGATCCGGATAACGGG | 98 |
| (sfGFP161-230 + MO- | GCATTGAACACCATAaGTtAaAGTAGTGACAAGTGTTGGCCATGG | |
| target) | AACAGACAGCTCCTCGCCCTTGCTCACCATatccctatagtgagtcgtatta | |
| | atttcgcggACGCCTGCA | |
| GFP ATG MO(for 5′) | ACAGCTCCTCGCCCTTGCTCACCAT ^{(Artificial nucleic acid(Morpholino sequence))} | — |
| Myc tag ATG MO(for 3′) | GCTCCATAGCTTTAAATCATGGGA ^{(Artificial nucleic acid(Morpholino sequence))} | — |
| PCR-primer-1 for enco- | ttgttggacgacccagacatc | 99 |
| Zebra-RNA | | |
| PCR-primer-2 for enco- | ATGGGGTATTTGAGGGTCAGG | 100 |
| Zebra-RNA | | |
| PCR-primer-1 for Cage- | CTGTTCCATGGCCAACAC | 101 |
| RNA | | |
| PCR-primer-2 for Cage- | TGATCCGGATAACGGG | 102 |
| RNA | | |

### 3. DNA Origami Construction and Assembly of T7 RNAP_{SNAPf}

The rectangle type DNA origami tile (10.4 bp/turn) was folded in 1×Tile buffer (20 mM Tris-acetate, pH 7.5, 10 mM Mg(OAc)2, and 1 mM EDTA). Typically, 40 nM single-stranded M13mp18 DNA (NEB) and 110 nM of each staple strand (3-fold excess) were mixed in 1×Tile buffer and annealed from 85° C. to 25° C. in a PCR machine (Bio-Rad) at an average rate of 1° C./30 s. The folded DNA origami was then loaded on a MicroSpin S-300 HR column (GE Healthcare), and the excess staple strands were removed. Then, DNA origami was mixed with Streptavidin (SA) (stoichiometric ratio 1:20); the mixed sample was then incubated for 10 minutes at 23° C., and the excess SA was removed by gel filtration spin column (MicroSpin S-400 HR column (GE Healthcare). Then, T7 RNAP_{SNAPf} was mixed with the DNA origami and purified by DNA-conjugated magnetic beads via a toehold displacement mechanism (Dynabeads MyOne Streptavidin C1, Thermo Fisher Scientific) with a publicly known method (T7-SA-chip. T. Torisawa et al., Autoinhibition and cooperative activation mechanisms of cytoplasmic dynein. Nat. Cell Biol. 16, 1118-1124 (2014)).

### 4. Anchoring of the First and Second Genes on the T7-Chip

The first gene was attached to the DNA origami tile using avidin-biotin method. The dual-biotin modified primer was used for PCR, and the resulting PCR product was used in either an unpurified or agarose-gel-purified form, which was concentrated if required. Then, dual-biotin attached gene was mixed with T7-SA-chip and used without purification. The second gene was integrated into the DNA origami tile by the repair method. First, dual-biotin attached second gene was mixed with Streptavidin (SA) (stoichiometric ratio 1:20); the mixed sample (SA-gene) was then incubated for 10 minutes at 23° C., and purified by 1% agarose gel electrophoresis. Purified SA-gene was concentrated to 200-250 nM by a vacuum pump (TOMY MV-100). Then, the origami tile was annealed with all staples except one. After the first gene was integrated, the folded DNA origami tile was incubated with the missing staples (stoichiometric ratio 1:5), which carried the dual-biotin tag at the 5′ end. The mixed sample was incubated for 1 hour at 50° C., and was then loaded on a MicroSpin S-400 HR column (GE Healthcare) to remove the excess missing staples. After the streptavidin pre-conjugated second gene was anchored onto the DNA origami tile, the sample was mixed with T7 RNAP_{SNAPf}, and the nano-chips were purified by the above-mentioned magnetic bead method. The yields of the samples were estimated by agarose gel electrophoresis and AFM. Agarose gels were imaged with an FLA-3000 image analyzer (Fujifilm).

### 5. Anchoring the Sensor-Attached Genes on the T7-Chip

The sensor-attached genes were anchored on the T7-chip by a similar method as described above. The inventors note that biotin was used instead of dual-biotin for some sensor-attached genes. Most of the sensor-attached genes except for those producing small RNA (miRNA) were incubated with the T7-chip (5 nM sensor-attached gene and 0.5 nM of T7-chip) for 30 min at 4° C. and used without purification. MicroRNA (miRNA)-producing logic-chips were purified by the above mentioned magnetic bead method. The inventors confirmed that the excess freely diffusing sensor-attached genes that had sfGFP ORF were not transcribed by the T7-chip (data not shown), as the apparent Km value of the T7-chip to the freely diffusing substrate-gene was on the micro-molar order (The inventors confirmed the micro-molar order of Km down to 200 bp substrate-gene length. Data not shown).

### 6. Preparation of Reprogrammable Nano-Chip

After the photoreactive staple was cross-linked internally by 365 nm UV irradiation for 30 min at room temperature, the cross-linked band was purified by gel electrophoresis (15% urea-PAGE, 300 V 60 min), and column purified (Qiagen nucleotide removal column). After being dried the cross-linked staple and dissolved in Tris-EDTA (TE) buffer, the photoreactive staple was mixed with other staples, and the nano-chips were made as described above. Logic reprogramming was achieved at 4° C. by UV irradiation of a different wavelength (312 nm) for 10 min.

### 7. RNA Production in Individual System (Living Organism)

Single-stranded scaffold DNA for cage (3614 nt) was prepared by two methods. First method is following a publicly known method (P. Shrestha et al., Confined Space Facilitates G-quadruplex Formation. Nat. Nanotechnol. 12, 582-588 (2017)). Commercially available single-stranded P8064 scaffold DNA was mixed with two complementary DNA (TAAGGGAAAATTAATTAATAGCGACGAT (for PacI) and TAGCCTTTGTAGATCTCTCAAAAATA (for Bg1II)), P8064 scaffold was digested with restriction enzymes (PacI/BglII) at two positions, obtaining single-stranded DNA of 3614 nt. In the second method, target region was amplified by asymmetric PCR following a publicly known method (Veneziano R et al., Enzymatic synthesis of gene-length single-stranded DNA. Sci Rep. 8, 6548 (2018); Forward primer: “p8064_F4475_20nt” TAATAGCGACGATTTACAGA, Reverse primer: “p8064_R24_23nt” TACAAAGGCTATCAGGTCATTGC”, PCR enzyme: Quanta, AccuStart Taq DNA Polymerase HiFi), and the single-stranded DNA was used after purification by gel electrophoresis. Single-stranded DNA obtained by asymmetric PCR was mainly used. The cage was assembled by a publicly known method (P. Shrestha et al., Confined Space Facilitates G-quadruplex Formation. Nat. Nanotechnol. 12, 582-588 (2017)). Typically, 20 nM single-stranded scaffold DNA and 80 nM of each staple strand (4-fold excess) were mixed in 1×Cage buffer (20mM Tris-HCl, pH7.6, 10 mM MgCl2, 1 mM EDTA). After 15 minutes incubation at 65° C., the cage was annealed at 50° C. for 1 hour in a PCR machine (Matsusada). The folded cage was then loaded on a MicroSpin S-200 HR column (GE Healthcare), and the excess staple strands were removed. RNAP and target gene were integrated as described above. Cage (˜5 nM) was microinjected into the zebrafish embryo. To prevent the degradation of nascent RNA, morpholino oligo (10 uM) that can bind to the terminal part of nascent RNA was mixed in the cage solution. After 1-hour incubation at 28° C., RNAs were extracted from the embryo through Trizol and DNaseI treatment. Then, product RNA was reverse transcribed with the kit (TAKARA PrimeScript RT Master Mix), and was quantified by SYBR green I using real-time PCR machine (Applied Biosystems, StepOne Realtime PCR system).

### 8. AFM Imaging

High speed AFM (Nano Explorer, RIBM) was used for imaging. The samples were diluted in AFM imaging buffer (20 mM Tris-acetate, pH 7.5, and 10 mM Mg(OAc)2), dipped on a mica surface, and imaged by tapping-mode following a publicly known method (O. I. Wilner et al., Enzyme cascades activated on topologically programmed DNA scaffolds. Nat. Nanotechnol. 4, 249-254 (2009)).

### 9. Transcription Assay

Transcription activity was assayed in transcription buffer (10 mM HEPES-KOH, pH 7.6, 10 mM MgCl2, 2.5 mM DTT, 0.5 mM spermidine, 1.25 mM of each NTP, 0.25 ug/ml PPlase, and 12.5 mM GMP) using [a³²P]-UTP (PerkinElmer, NEG007H) at 37° C. The transcripts were analyzed with PAGE and imaged with BAS-5000 (Fuji-film). The transcripts were quantified with a calibration curve generated using spotted serial dilutions of [a³²P]-UTP.

### 10. Transcription—Translation Coupled Assay in the PURE System

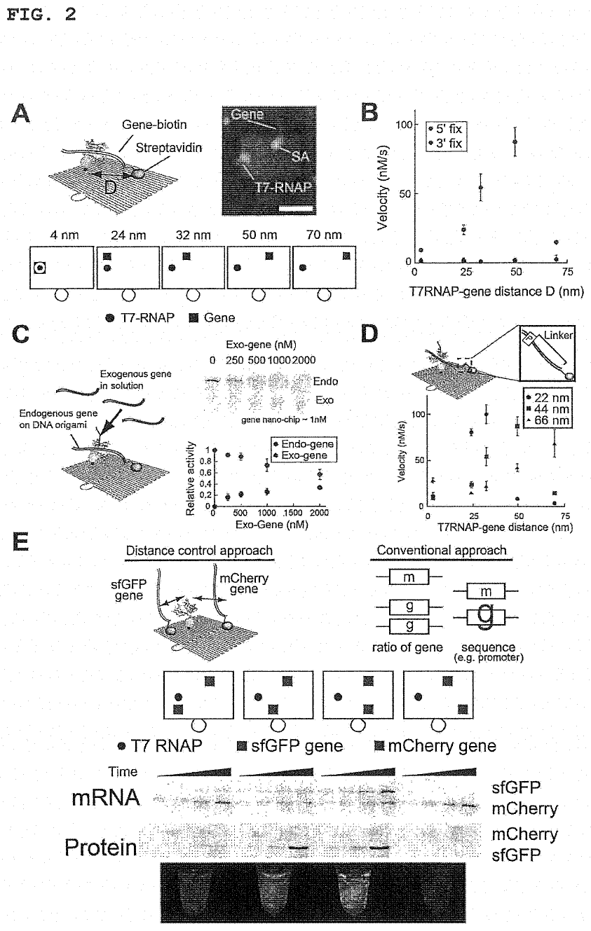

The reaction was performed in PUREfrex 2.0 at 37° C. For the nano-chip assay using T7-chip or T7-gene-chip, freely diffusing T7 RNAP was omitted and the nano-chip was added at each time of reaction. The transcripts were analyzed as described above. Protein production was measured by RI or by the fluorescence intensity of sfGFP and/or mCherry. For the RI analysis, [³⁵S]-Met was used and the protein was analyzed with SDS-PAGE and imaged with BAS-5000. For the fluorescence intensity analysis, a qPCR machine (StepOne, ABI) was used as a time-lapse incubator. For the data shown in FIG. 2E, the reaction mixture was excited by an LED (Light Engine, Lumencor) using the excitation filters, FF01-475/35 and FF01-482/563-25 (Semrock), and the excitation light source was reflected by a dichroic mirror (Di01-R488/561, Semrock), and the fluorescence image was captured by an iPhone (registered trademark) 5.

### 11. Microfluidic Devices for Water-in-Oil Droplet Formation

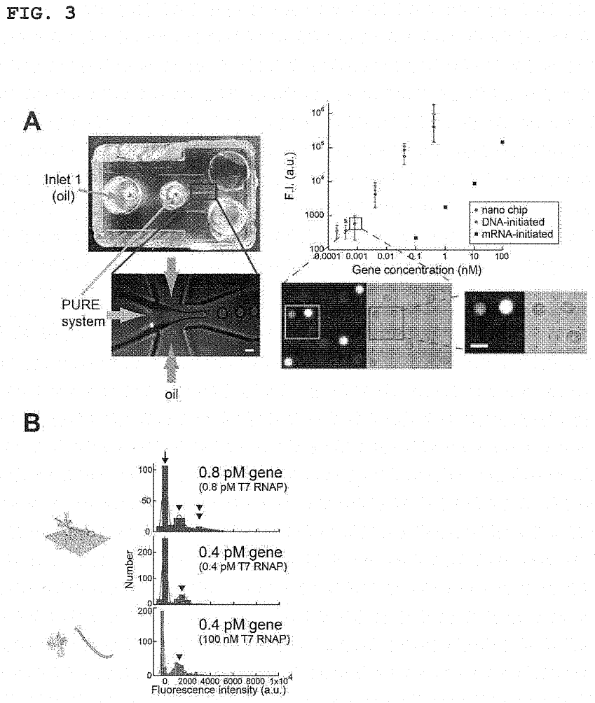

Polydimethylsiloxane (PDMS) devices were prepared using standard soft-lithography and mold-replica techniques. The device consists of two inlets (one for oil solution and one for aqueous solutions), and one outlet and a flow-focusing junction (FIG. 3A). The widths of the main channels and the flow-focusing constriction were 100 and 40 um, respectively. The height of all channels was 50 um. The surface of the microfluidic device channels was coated with (Heptadecafluoro-1,1,2,2-tetrahydrodecyl) dimethylchlorosilane, Gelest, SIH5840.4) to prevent wetting of the drops on the channel walls. PDMS was purchased from DOW CORNING TORAY (SILPOT 184W/C).

### 12. Construction of the Water-in-Oil Droplet and Observation of the Transcription-Translation Activity

Water-in-oil (w/o) droplets were generated using the above PDMS-based microfluidic devices. Briefly, the PDMS devices were filled with oil (98% v/v of Mineral oil (Sigma, M5904) and 2% v/v of ABIL EM90 (Cetyl PEG/PPG-10/1 Dimethicone, EVONIK)), in one inlet (inlet 1 in FIG. 3A) and aqueous solution (the PURE system solution containing gene-substrates) in the other inlet. At 20° C., the solutions were flowed by air pressure at 40 kPa for oil and 20 kPa for the PURE system to generate w/o droplets with a diameter of 20 um and a volume of 4 pL at 100 Hz (10² droplets /s). The droplet formation process was monitored with a high-speed camera (DigiMo, LRH2500XE), and the pressure was manually adjusted with the assistance of a homemade Visual Basic .NET 2010 (Microsoft) program. To prevent the non-specific binding of the PURE system components, excess amino acids (f. 8 mM) were supplemented into PUREfrex 2.0 and the surface of the PDMS was further coated with Blockmaster (registered trademark) PA651 (JSR Life Sciences Corporation). In addition, to improve the reproducibility of the data, the T7 RNAP concentration was increased to 100 nM for the DNA (PCR products)—initiated experiments using the freely diffusing T7 RNAP enzyme. After 3 h incubation at 37° C. in the PDMS devices, samples were pipetted out and dropped onto a quartz cover glass (7980 standard grade, Corning), and observed under inverted type fluorescence microscope (IX-70, Olympus). Fluorescence images of sfGFP and mCherry were separated using a W-View (Hamamatsu Photonics) and then projected side-by-side onto an electron-multiplying charge coupled device camera (iXon Ultra 897, Andor). Images were captured at a frame rate of 1-10 frames per second with an EM-gain of 0-100, and analyzed using ImageJ. A calibration curve was constructed using sfGFP and mCherry proteins purified from E. coli.

### 13. Prediction of the Enzyme-Substrate Collision Efficiency Curve

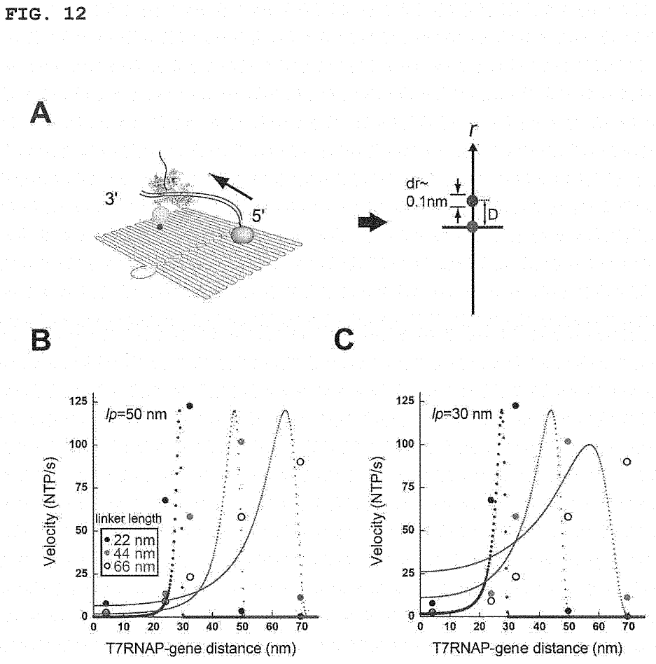

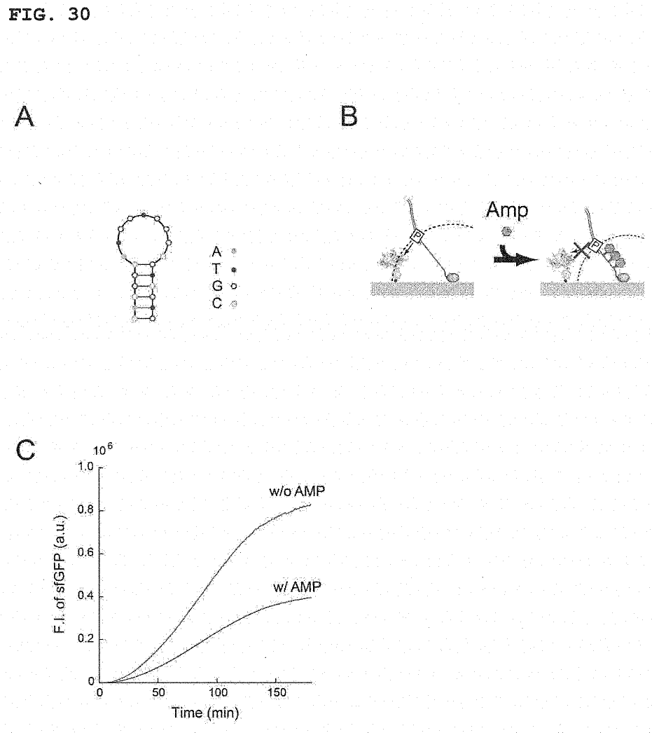

To predict the collision efficiency, the inventors only considered the effect of bending, although the DNA rigidity is determined by three parameters: extension, bending and torsion (Y. Miyazono, M. Hayashi, P. Karagiannis, Y. Harada, H. Tadakuma. Strain through the neck linker ensures processive runs: a DNA-kinesin hybrid nanomachine study. EMBO J. 29, 93-106 (2010). For the calculation, the inventors assumed that the DNA ends behave according to the Worm-Like Chain (WLC) model (Eq S1) (D. Thirumalai, B. Y. Ha. Theoretical and Mathematical Models in Polymer Research, ed. Grosberg, A. (Academia, New York), pp. 1-35 (1988)), which has been observed experimentally for polyproline residues (B. Schuler, E. A. Lipman, P. J. Steinbach, M. Kumke, W. A. Eaton. Polyproline and the “spectroscopic ruler” revisited with single-molecule fluorescence. Proc Natl Acad Sci USA 102: 2754-2759 (2005)), as described by

$$\begin{matrix}Formula I & \\ P ( r ) = \frac{4 π {Cr}^{2}}{{{L}^{2} [ 1 - {( r / L )}^{2} ]}^{9 / 2}} exp ( - \frac{3 L}{4 {l}_{p} [ 1 - {( r / L )}^{2} ]} ) & 〈 Eq . S1 〉\end{matrix}$$

Wherein r: the distance between the ends of a DNA, L: the is DNA contour length, P(r): the probability of the DNA with r, lp: the (bending) persistence length of DNA, C: a normalization factor. For simplicity, the inventors considered a one-dimensional distribution assuming that lp=50 nm (FIG. 12B) and 30 nm (FIG. 12C).

### Example 1

### Example 1

### Preparation of Enzyme-Substrate Complex

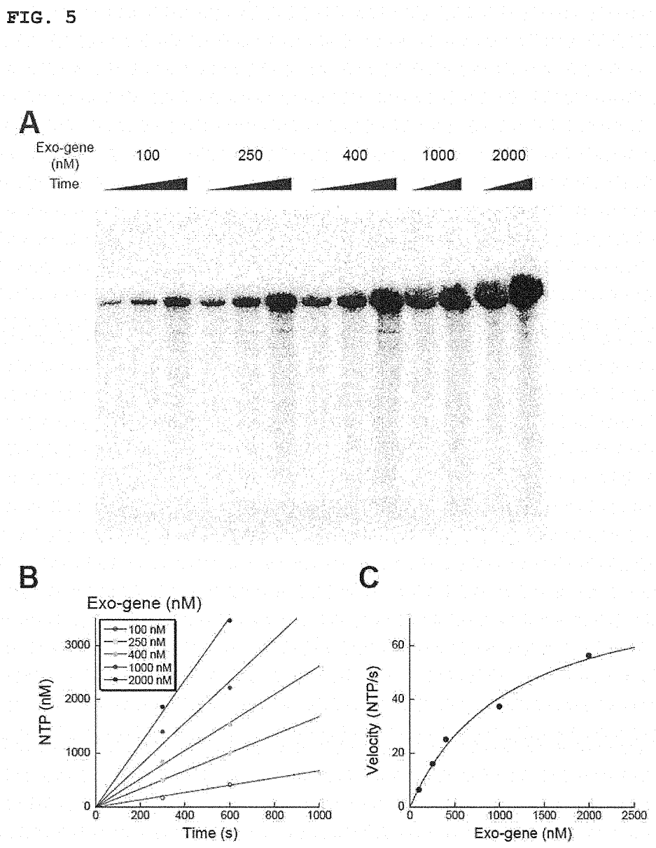

The inventors used Rothemund's rectangle DNA origami as the scaffold for our enzyme-substrate complex. The inventors first covalently attached a SNAPf-tag protein fused to the T7 RNA polymerase (T7 RNAP_{SNAPf}) onto the handle extruding from the DNA origami, which had an attached SNAP-tag ligand (FIG. 1A). Attachment of the T7 RNAP_{SNAPf} was confirmed by gel analysis (FIGS. 1B and C) and atomic force microscopy (AFM) imaging (FIG. 1D), showing a high yield (−95%) of the T7 RNAP_{SNAPf} linked DNA nanostructure (T7-chip). The transcription activity of the T7-chip exhibited Michaelis-Menten type kinetics (FIGS. 1E, F and FIG. 5). However, the apparent Km value for the externally diffusing substrate gene was approximately 200-fold higher than that for the unanchored, freely diffusing T7 RNAP_{SNAPf} (FIG. 1F, 1,000 nM for the T7-chip, 5.6 nM for the freely diffusing T7 RNAP_{SNAPf} for transcription of the 942 nucleotides sfGFP gene), indicating a low affinity for externally diffusing substrate genes. Additionally, the Vmax of the T7-chip was lower than that of the freely diffusing T7 RNAP_{SNAPf} (FIG. 1F), with values of 34 and 62 nM/s, respectively. Taken together, these data show that the T7-chip has low affinity for externally diffusing substrate genes.

### Example 2

### Example 2

### Integration of a Target Gene onto the T7-Chip Through an Avidin-Biotin Interaction

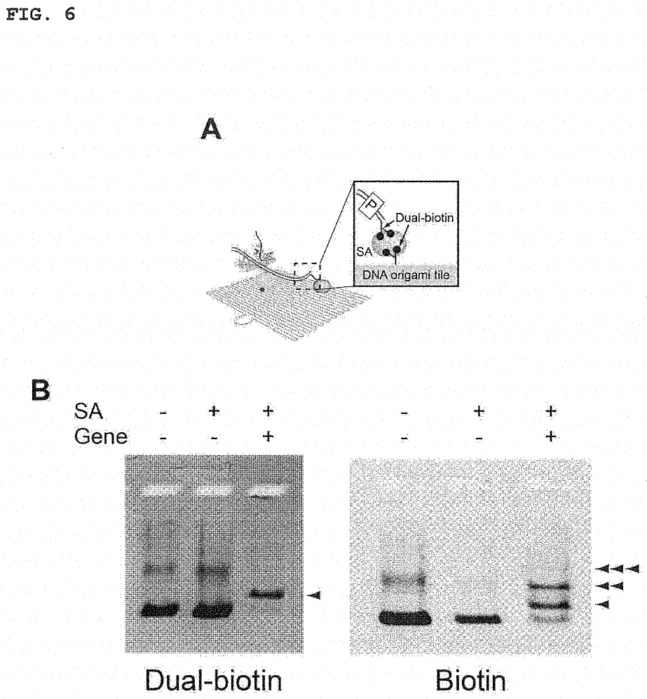

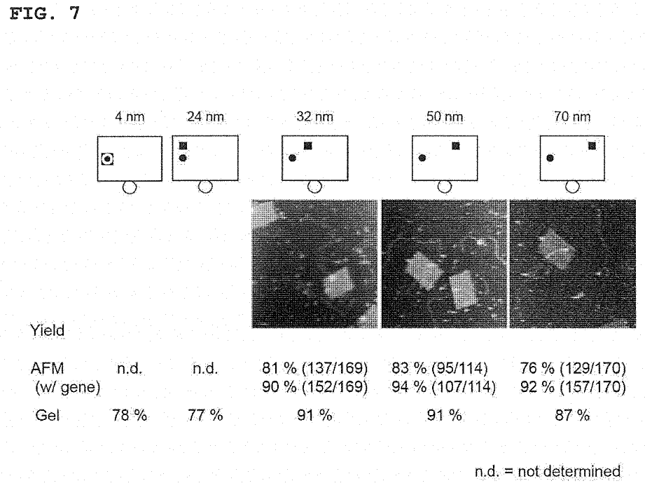

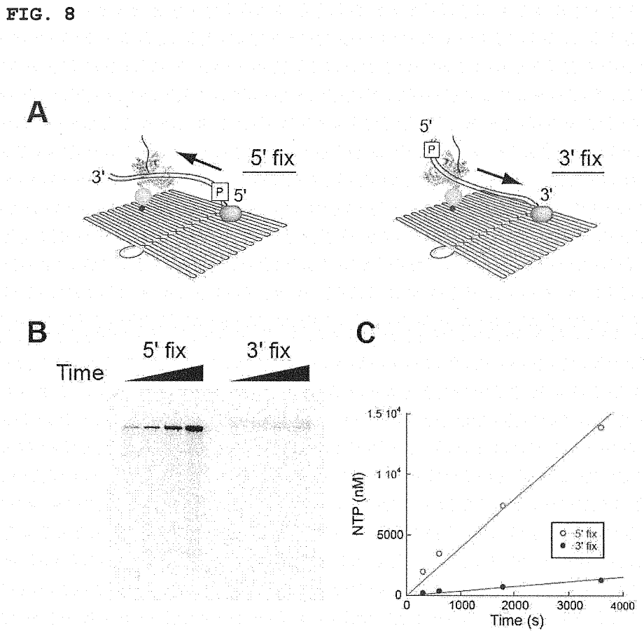

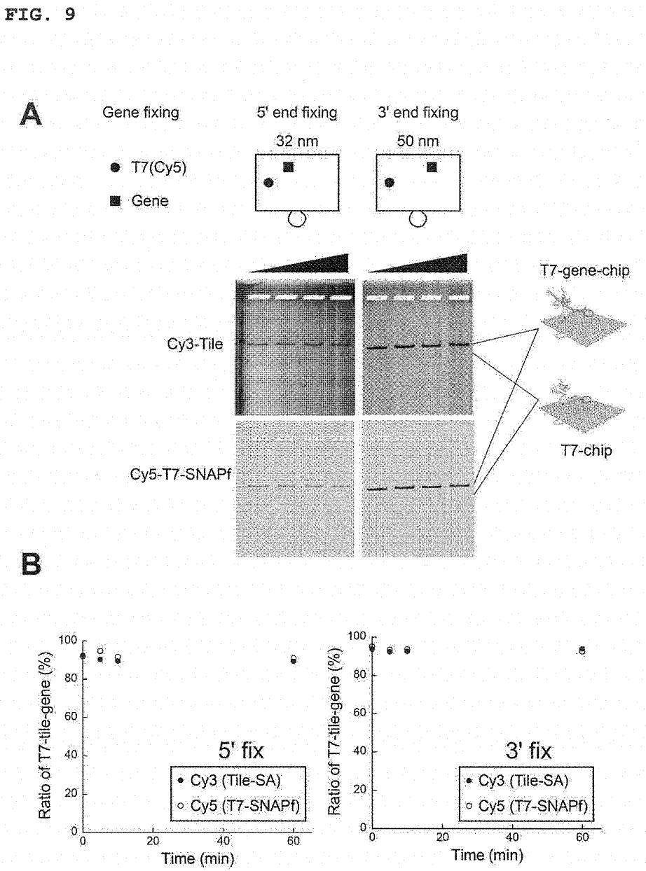

The inventors next integrated a substrate gene onto the T7-chip through an avidin-biotin interaction (T7-gene-chip, FIG. 6). The inventors used the advantage of DNA origami, in which the molecular layout can be controlled with nanometer precision. For the redox cascade enzymes, such as glucose dehydrogenase (GDH) and malic dehydrogenase (MDH), an intermolecular distance dependency in channeling the intermediate substrate, NAD, has been observed. The inventors speculated that a high-molecular-weight polymer substrate would also show a dependence on the intermolecular distance between the enzyme, RNAP, and the substrate gene. In addition, there should be some differences attribute to the polymer-substrate properties such as the tethered direction, i.e., 5′ end vs. 3′ end, and the length of the linker between the anchoring points and the promoter sequence. The inventors used 5 sites for substrate-gene anchoring, which were positioned such that the intermolecular distances between the RNAP and the substrate genes would be 4, 24, 32, 50, and 70 nm (FIG. 2A). The yield of the 5 complexes was in the range of 69-83% (FIG. 7). The inventors compared the transcription activity of the gene tethered at the 5′ end vs. the 3′ end, with the promoter sequence near the 5′ end (44 nm away from the 5′ end). The inventors found that tethering the 5′ end resulted in much higher transcription activity compared to tethering the 3′ end (FIG. 2B, FIGS. 8 and 9), which suggested that the aim (linker) length, the length from the anchoring point to the promoter sequence, and the subsequent collision efficiency between the promoter sequence and the enzyme might affect the transcription activity. The activity of the 5′ end tethered T7-gene-chip showed a peak at approximately 50 nm, which is in sharp contrast to that reported for the low-molecular-weight intermediate substrate NAD. NAD was fixed on the DNA origami through a single-stranded DNA (ssDNA) anchor. The activity increased as the distance between the GDH enzyme and the NAD substrate decreased and the proximity effect diminished as the intermolecular distance increased from 0 nm to 10-20 nm (O. I. Wilner et al., Enzyme cascades activated on topologically programmed DNA scaffolds. Nat. Nanotechnol. 4, 249-254 (2009).; J. Fu et al., Interenzyme substrate diffusion for an enzyme cascade organized on spatially addressable DNA nanostructures. J. Am. Chem. Soc. 134, 5516-5519 (2012).; J. Fu et al., Multi-enzyme complexes on DNA scaffolds capable of substrate channelling with an artificial swinging arm. Nat. Nanotechnol. 9, 531-536 (2014)). This result indicates that the rigidity of the anchor arm (linker), i.e., double-stranded DNA (dsDNA) vs. ssDNA for the T7-gene-chip and the GDH-NAD pair, strongly affects the collision efficiency of the promoter sequence to the enzyme and the subsequent enzymatic activity.

### Example 3

### Example 3

### Measuring the Kinetic Properties of the Nano-Chip

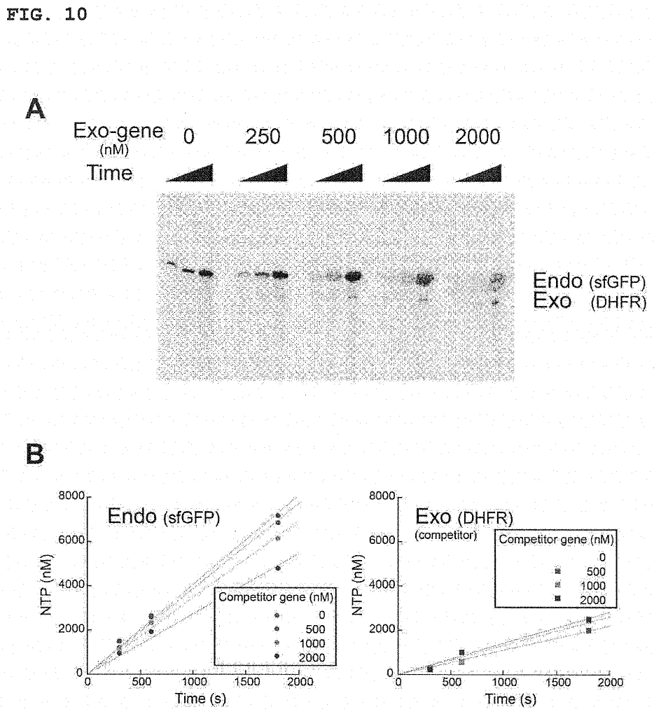

The inventors next measured the kinetic properties of the nano-chip under semi-optimal conditions. The inventors first estimated the effective concentration of the endogenous gene integrated on the T7-gene-chip via a competition assay, assuming that the collision efficiency of the endogenous gene with T7 RNAP_{SNAPf} corresponds to the effective concentration. The inventors estimated that the effective concentration of the integrated gene was more than 2 uM based on a competition assay with an approximately 1 nM T7-gene-chip and 0-2,000 nM DHFR gene as an exogenous-competitor (FIG. 2C and FIG. 10).

### Example 4

### Example 4

### Confirmation of the Orthogonality of the Nano-Chip

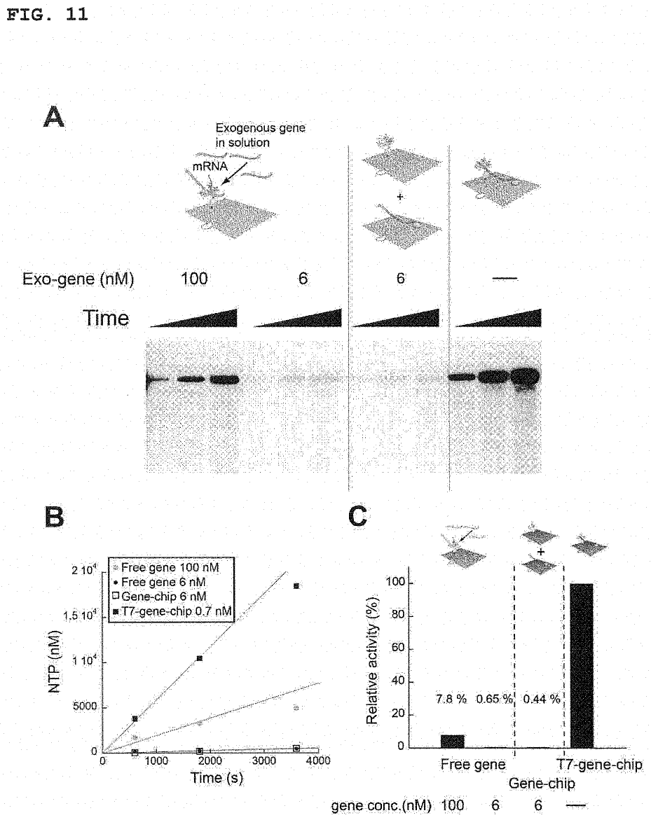

The inventors also confirmed the orthogonality of the nano-chip. The inventors prepared two types of nano-chips that were integrated with only RNAP (T7-chip) or substrate genes (gene-chip) and observed little transcription, indicating the orthogonality of the substrate gene (FIG. 11). The Vmax value of the 5′ tethered T7-gene-chip was 87 nM/s that was higher than that of the freely diffusing T7 RNAP_{SNAPf} (62 nM/s). This result may reflect enhancement in the activity of the enzyme tethered to the polyanionic surface through the action of a strongly bound hydration layer or other mechanisms (e.g., the pH near the surface is lower than that in the bulk).

### Example 5

### Example 5

### Explore the Effect of the Intermolecular Distance

The inventors further explored the effect of the intermolecular distance upon activity by changing the distance between the RNAPs and the substrate gene fixed part (anchor part), as well as the length of the linker between the substrate gene attachment points and the promoter sequence (FIGS. 2D and E). The inventors found that with different linkers, the intermolecular length dependence yielded different peak profiles, which roughly obeyed the predicted collision efficiency curve assuming a worm-like chain model of DNA (FIG. 12). Given the transcriptional activity changes with varying intermolecular distances, the inventors expected that the expression output of the two genes could be controlled by changing the molecular layout (FIG. 2E), where the intermolecular distance between the RNAPs and two genes, sfGFP and mCherry, was controlled. The transcription and subsequent translation activity of the T7-gene-chip in a cell-free translation system, the PURE system31, clearly demonstrated the relative intermolecular distance dependence of the two genes by their expression ratios (FIG. 2E and FIG. 13), indicating that the rational design of multiple gene expression modules could be achieved by controlling the respective intermolecular distances. Taken together, these data show that the nano-layout ability of DNA origami technology allows us to rationally design the orthogonal transcription nano-chip.

A key challenge of synthetic biology is to build up complex genetic circuits within a confined cell-sized reaction volume, as it is difficult to completely avoid crosstalk between circuits. In electrical computer engineering, combinations of logic-chip units are used to form sophisticated large-scale circuits (e.g., LSI or Large Scale Integration), where each subset of logic circuits is localized to a confined area. If there are orthogonal unit elements that can sense a signal, execute a logic operation and produce an output flow within a confined chip, an approach similar to that in electronics may also be possible in biological genetic circuits. This approach may be possible using DNA origami technology, which can integrate all the required components on the same nano-chip, and can achieve the rational design of an orthogonal transcription nano-chip.

### Example 6

### Example 6

### Confirmation of the Nano-Chip's Ability to Function as a Single Unit Element

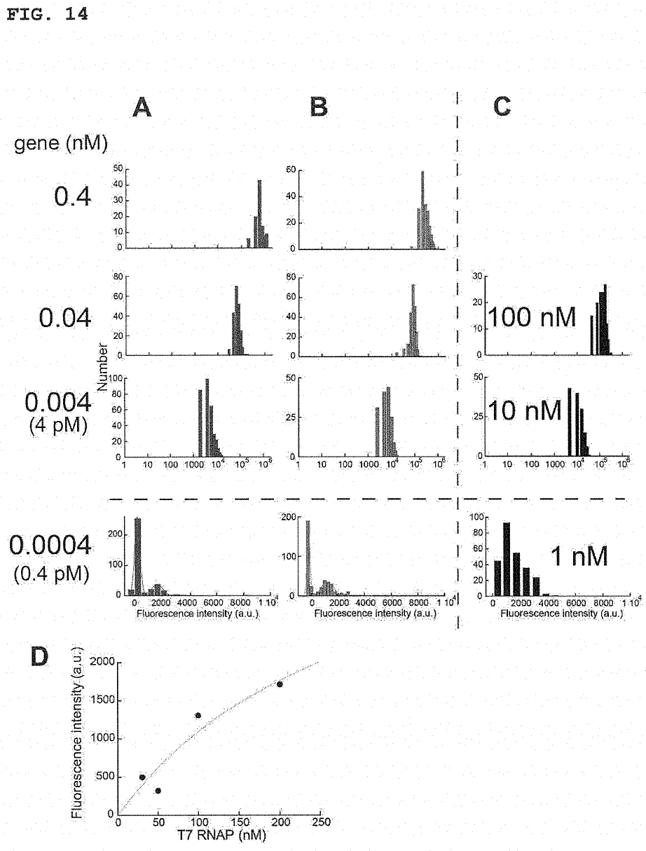

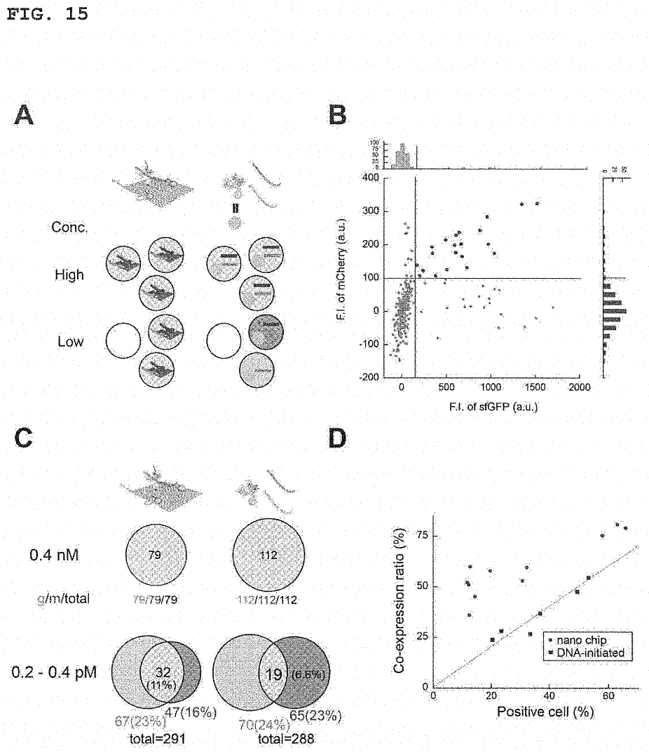

The inventors first confirmed whether the nano-chip could work as a single unit element by measuring the activity of extremely diluted nano-chips. To this end, the inventors encapsulated the nano-chips in water-in-oil (w/o) droplets containing the PURE cell free translation system, which mimics artificial cells. The inventors compared the gene expression activity of 1) the integrated nano-chip system to the activities of diffusion reaction-based systems initiated by either 2) DNA or 3) mRNA, in water-in-oil (w/o) droplets (FIG. 3A). The inventors then decreased the concentration to a single-chip level. The inventors found that decreasing the concentration of the nucleic acid substrates reduced the overall protein production in all three systems, as expected (FIG. 3A and FIG. 14), but the mRNA-initiated reaction required a three orders of magnitude greater substrate concentration to produce a similar level sfGFP protein to the other two systems. This difference likely reflects the fact that gene expression is amplified only once at the translation step in the mRNA-initiated reaction, whereas the other two systems undergo additional amplification at transcription. By further reducing the gene concentration, the fluorescence of each (w/o) droplet showed a heterogeneous intensity, and the number of dark cells increased with decreasing gene concentration (FIGS. 3A and B). At 0.8 pM nano-chip concentration, the fluorescence intensity distribution of each cell showed distinctive peaks quantized at intervals of approximately 1,500 (a.u.). At 0.4 pM the number of peaks decreased to one, where most of the cells were dark, and only sparse cells showed a peak at 1,450±117 (a.u.) (n=3, corresponding to 12 nM sfGFP molecules), indicating that the latter peak corresponds to (w/o) droplets having a single molecules of nano-chip. The data for the freely diffusing DNA gene also showed a peak at approximately 1,310±466 (a.u.) (n=3) with 100 nM freely diffusing RNAPs (FIG. 4B), showing that the activity of a single nano-chip is equivalent to that of 100 nM freely diffusing RNAPs, i.e., 10⁴-10⁵ molecules in the 20 um diameter artificial cells (FIG. 14). These results, together with the bulk experiments showing the high transcription activity of the nano-chip (FIGS. 1F and 2B), indicate that the nano-chip efficiently transcribes the substrate gene in bulk solution and in artificial cells. The inventors also confirmed that all required components could be integrated on the same nano-chip using a two-gene co-expression assay (FIG. 15).

### Example 7

### Example 7

### Confirmation of the Nano-Chip's Ability to Function as a Logic Chip

After confirming the nano-chip's ability to function as a unit element, the inventors further examined whether the nano-chip could function as logic-chip that could sense a signal, execute a logic operation and produce an output flow. To this end, the inventors integrated a logic gate on the nano-chip, combining a sensor and an actuator (RNAP enzyme) on the same chip (FIGS. 16 and 17). Thus, the sensed signals are computed and then directly converted into the output.

### Example 8

### Example 8

### Reprogram Ability of the Logic Chip

After confirming the nano-chip's ability as a logic chip, the inventors examined whether the logic of nano-chip was reprogrammable. To this end, the inventors integrated photoreactive cross-linking nucleotide containing staple and combined the sensor (FIG. 4). With this approach, the integrated sensor's logic function could be reprogrammed by UV light.

### <Three Crucial Factors for Sensor Design>

In this working example, the inventors used sensors comprising a nucleic acid-based biopolymer. Thus, three crucial factors must be considered (FIG. 17A).

### 1) Rigidity of the Sensor

The sensor's rigidity determines the inter-molecular distance dependency of the collision efficiency between the enzyme and substrate. Assuming a worm-like chain (WLC) model of DNA, the inventors' data showed that the dsDNA linker largely obeyed the predicted collision efficiency curve.

### 2) Scanning Ability of RNAP

RNAP can interact with other sequences in sites outside promoters region and can walk along dsDNA for a long distance. This characteristic may widen the area accessible by RNAP beyond the value predicted based on the rigidity of the sensor (linker).

### 3) Pulling Force of RNAP

In the integrated system, the sensor and RNAP are anchored to the rigid scaffold, providing internal tension (force) to both. Specifically, pulling (stretching) force was applied to the sensor, and assist force was applied to RNAP. To evaluate the effects of pulling and assist force, the inventors examined each in turn. Regarding the pulling force, the inventors considered that, even in the absence of the signal (miRNA), some of the “ON switch” had leak activity greater than that calculated from the equilibrium constant of the sensor's stem loop (see also FIG. 18D). For example, when the free-energy change (ΔG) in the secondary structure formation of the sensor is ˜−5 kcal/mol, the equilibrium constant K=exp(−ΔG/RT) is on the order of thousands (˜3400), causing that most of the sensor to adopt a folded state. However, at ΔG˜−5 kcal/mol, the sensor was half activated, even in the absence of the miRNA signal. The inventors speculated that this leakage could be explained by the effect of pulling (stretching) force and therefore considered this effect using ΔG as an indicator. Regarding assist force, the inventors considered the fact that the RNAP has low load-dependency at a high NTP concentration, whereas the kinesin motor protein that walks along the microtubule has a high load-dependency. Thus, for nano-chip design, the inventors considered only the effect of pulling (stretching) force, not of assist force on RNAP.

### <Effect of Pulling (Stretching) Force on the Effective Sensor Arm Length>

In this working example, to detect the miRNA profile, the inventors designed sensors that have an ssDNA domain and a dsDNA domain (FIG. 17B). The former ssDNA domain provides a miRNA-binding site, and the latter dsDNA domain serve as a rigid spacer. Without tension, the free-end of the ssDNA, which is next to the dsDNA linker and thus closer to the promoter region, should be near the other end (fixed-anchored-end) of the ssDNA, because the ssDNA behaves as a soft spring. However, as mentioned above, the inventors thought that the scanning ability and pulling force of RNAP would stretch the linker length; thus, the free-end and promoter region could access a wider area than the one predicted from models of a high molecular weight polymer (e.g., the WLC model). The inventors therefore determined a simplified empirical rule experimentally.

Upon miRNA binding, the miRNA-binding sites of the linker formed a DNA-RNA hybrid double strand: dsD/RNA (see also FIG. 18A). Assuming dsD/RNA has physical properties similar to those of dsDNA, the resulting 21-22 bp dsD/RNA, which is shorter than the persistence length (150 bp) of dsDNA, can be considered as a rigid rod. For simplicity, the inventors made the following assumptions. First, despite ssDNA being located at both dsD/RNA ends, the inventors assumed that ssDNA exists only at one end. Additionally, the inventors assumed that the double strand part of the linker is a rigid rod and that the single strand part being stretched by RNAP possesses a constant average length per nucleotide.

Considering all the above assumption, the effective length of the sensor arm, which is defined as the linker part starting from the anchor part and ending at the promoter sequence, for the ON and OFF state is defined as below:

Formula 2

L_{off}=C_{dsDNA}×N_{dsDNA}+C_{ssDNAoff}×N_{ssDNAoff} <Eq. S2>

Formula 3

L_{ON}=C_{dsDNA}×(N_{dsDNA}+N_{dsD/RNA})+C_{ssDNAon}×N_{ssDNAon} <Eq. S3>

(wherein
C_{dsDNA}: constant value, here 0.34 (nm/bp)
C_{ssDNAoff}: undetermined constant for ssDNA in the OFF state
C_{ssDNAon}: undetermined constant for ssDNA in the ON state (stretched by RNAP)
N_{dsDNA}: Number of base pairs in the sensor's dsDNA part
N_{dsD/RNA}: Number of base pairs in the dsD/RNA, here N×(21-22) (N: Number of miRNA-binding sites)
N_{ssDNAoff}: Number of bases in the sensor's ssDNA part in the OFF state
N_{ssDNAon}: Number of bases in the sensor's ssDNA part in the ON state)

To predict the effective sensor arm length, the inventors determined the undetermined constants of C_{ssDNAoff} and C_{ssDNAon}. The inventors first considered the C_{ssDNAoff}. Without tension, the value of C_{ssDNAoff} would be small; however, when stretched by RNAP, the C_{ssDNAoff} value might be large. To predict the effective sensor arm length, the maximum arm length must be estimated. Therefore, the inventors attempted to determine the C_{ssDNAoff} under tension, which could approximate C_{ssDNAon} under tension. Thus, the inventors introduced a new constant, C_{ssDNA}, that is equivalent to C_{ssDNAoff} and C_{ssDNAon} under tension. The inventors measured the chip activity with a fixed inter-molecular distance between the enzyme and substrate (50 nm, FIG. 17C) while changing the length of the linker comprising ssDNA (0-60 nt) and dsDNA (25-85 bp). With identical dsDNA linker lengths, the sensor arm with a small ssDNA linker is too short to activate the chip activity, whereas the sensor arm with an long ssDNA linker is sufficiently long for activation (FIG. 17C). If the inventors assumed the tipping point arm length for the OFF-to-ON transition, the obtained data set with a different ssDNA/dsDNA combination might show a similar activity profile along the estimated sensor arm length. Using this assumption, the inventors estimated that the value of C_{ssDNA} under tension was approximately 0.23 nm/nt under our experimental conditions (FIG. 17D). The data also suggested that the ON/OFF ratio could reach up to several hundred when there is no leakage of sensor activity.

Hereafter, to design the sensor, the inventors estimated the sensor arm length using 0.23 nm/nt and 0.34 nm/bp for ssDNA and dsDNA, respectively.

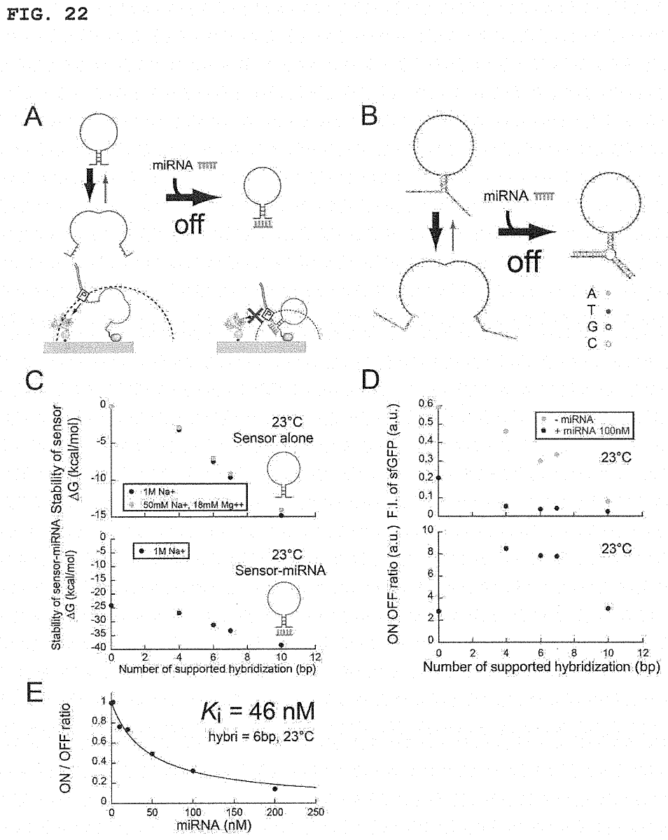

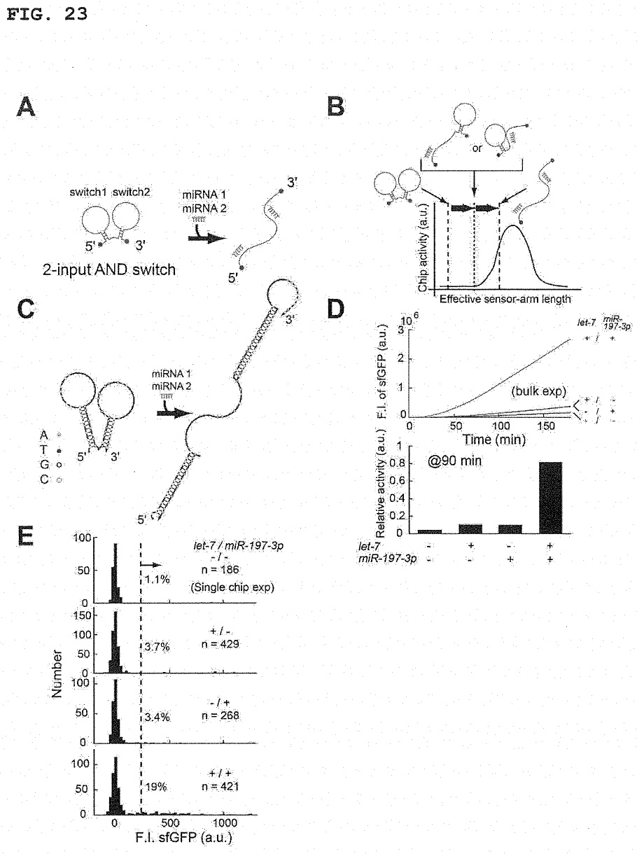

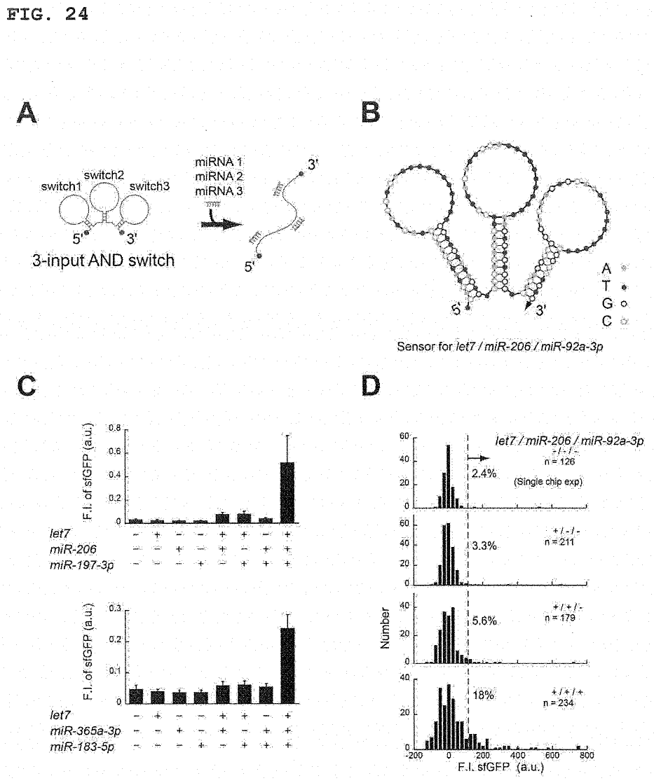

For sensor design, the inventors took advantages of the nano-chip, where the rational design of a gene expression platform could be achieved by controlling the intermolecular distances between the enzymes and genes. For example, when there was no signal (e.g., miRNA), the promoter sequence of the “ON switch,” which was made up of a stem and a loop, could not reach the RNAP. Upon signal hybridization, a toehold exchange occurred, and the reach length of the “ON switch” arm was sufficiently extended to initiate transcription (FIG. 4A and FIG. 18-21). A similar approach to controlling intermolecular distances, but in the reverse direction, created an “OFF” switch (FIG. 22). The orthogonality of the “ON switch” allowed the construction of “AND switches” by simply connecting two or three different “ON switches” in tandem (FIGS. 4B, C and FIG. 23-24), which is a function that works at the single chip level (FIGS. 23 and 24). In synthetic genetic circuits, three input “AND switch” requires six sub-switches and a total of 47 factors, including many molecules, making it difficult to further combine such building blocks to create computing circuits (Nielsen et al., Science. 352, aac7341, 2016). Although the input signals and working environments are very different, a single molecule of nano-chip logically functions as an equivalent to the complicated genetic circuits of 47 factors, suggesting that the synthetic genetic circuit has the potential to be highly simplified.

### <Structure of the ON Switch>

To maximize the effective sensor arm difference, the inventors used a stem-loop structure (FIG. 18A), with which the end-to-end distance of the sensor arm is short (˜a few nm) in the OFF state and then, upon miRNA hybridization-induced deploying, becomes sufficiently long to activate transcription. There are five domains: toehold (T), hybridization (H), loop (L), anti-hybridization (H*) and the spacer between the stem-loop and the other part of the linker. Small RNA (miRNA), which binds to the Toehold and Hybridization domains (FIG. 18B), first binds to Toehold domain and then penetrates into the stem structure and finally deploys the stem-loop. The Loop domain comprising a polyT sequence contributes to increasing the affinity for miRNA in the OFF/folded state (before miRNA hybridization), and the reach length of the sensor arm in the ON/deployed state (after hybridization). The inventors further introduced “support hybridization” at the root of the stem structure to support stable hybridization. The spacer was introduced to prevent steric hindrance affecting miRNA binding to the sensor.

### <Key Factors for ON Switch Design>

As the inventors used the stem-loop structure for the ON switch design, there are key factors for each ON and OFF state of the switch. For the ON state, where the sensor arm is extended by miRNA hybridization, sufficient extension of the sensor arm to ensure efficient collision of the promoter sequence to RNAP is important. Thus, the reach length (effective end-to-end distance) of the sensor arm, which comprises the dsDNA and ssDNA linker domains (FIG. 17B), is crucial. As short dsDNA can be considered as a rigid rod with a constant length, whereas the ssDNA linker domain transforms and extends upon miRNA hybridization, the effective length of the ssDNA linker domain is the key factor. For the OFF state, where the stem-loop structure takes a folded form, the stem structure stability is crucial for preventing leak activity and simultaneously maintaining sufficient degrees of freedom of the loop part for miRNA binding. Thus, the ΔG in the secondary structure formation of the sensor and the length of the loop are the key factors.

### <Sensor Arm Length>

The inventors considered the sensor arm length, which comprises the dsDNA and ssDNA linker domains. As mentioned above, a short dsDNA linker domain can be considered as a rigid rod with a constant length, whereas the ssDNA linker domain is extended upon miRNA hybridization. Therefore, the main contribution of the effective sensor arm length difference in the OFF-to-ON-state transition is attributed to the change in the ssDNA linker domain (FIG. 18C). Thus, to determine the first trial length of the dsDNA and ssDNA linkers, the inventors considered the ssDNA linker domain first.

For ssDNA linker domain length, the inventors could consider the two limits: minimum required length and maximum available length. However, as the inventors could compensate for the shortage (or excess) of the effective sensor arm length by lengthening (or shortening) the dsDNA linker domain (FIG. 17D), the inventors considered the maximum available length here. The experimentally available nucleotide length is limited by 1) the maximum nucleotide length of a commercial DNA synthesis service (N_{service}, 90 nt for the ON switch), and 2) the maximum nucleotide length of the PCR primer for the sensor-attached substrate-genes (Nₘₐₓ₋ₚᵣᵢₘₑᵣ, 31 nt for the ON switch). Thus, the maximum available ssDNA linker domain in this working example was 59 nt (=90−31).

After setting the first trial length of the ssDNA linker domain to 59 nt, the inventors considered the remaining part of sensor arm length, the dsDNA linker domain (FIG. 17B). Considering the experimental results using a polyT sequence as the ssDNA linker (FIG. 17D), the inventors chose a first trial length of the dsDNA linker of 45 nt, as the data of the 65 nt dsDNA linker (FIG. 17D) covered most of the OFF-to-ON transition upon ssDNA linker lengthening, and the number of the double strand part of the sensor arm after miRNA hybridization was 67 nt with a 45 nt dsDNA linker (N_{dsDNA}+N_{dsD/RNA}=45+22=67 nt).

In the remaining working example of the ON switch, the inventors used a sensor arm comprising a 59 nt ssDNA linker domain and a 45 nt dsDNA linker domain.

### <Optimizing the Length of Each Sensor Domain>

Using a 59 nt ssDNA linker domain, the inventors next optimized the length of each of the 5 domains (Toehold (T), Hybridization (H), Loop (L), Anti-hybridization (H*) and spacer). As the stability of the stem structure and the kinetics of toehold exchange were highly depended on the ΔG of secondary structure formation of the sensor and on the effective loop length (T+L≡L′), the inventors mainly considered the Hybridization length (H) and effective loop length (L′) in this section. Furthermore, to maintain the toehold length as long as possible, the inventors introduced 1 bp of support hybridization at the root of the stem (FIG. 18B) and evaluated the chip activity by changing the effective hybridization length H′ (=H+1). The inventors named the primers (Table 1-7) using this H′ and effective loop length L′ (=T+L). For example, H12L31 has 12 nt of H′ and 31 nt of L′ (H=H′−1=12−1=11 nt, T=(T+H)−H=21−11=10 nt (Note: let-7 is 21 nt), L=L′−T=31−10=21 nt).

Using NUPACK software (http://www.nupack.org/), the inventors calculated the ΔG and found that a longer H′ provided a larger negative ΔG value (FIG. 18D). The activity of the chip depended on ΔG (FIG. 18D). Specifically, for a higher ΔG (i.e., a value close to zero) and a less stable stem-loop, the activity of the chip was similar in the presence or absence of miRNA signal. The leak (OFF) activity of the chip decrease as the ΔG decreased (larger negative value), with the leak activity remaining constant at ΔG<−6 kcal/mol. By contrast, the ON activity of the chip was constant for most ΔG values but slightly decreased at a low ΔG˜−13 kcal/mol, suggesting that at a large negative ΔG value, the stem stability is too strong and thus hinders miRNA hybridization. Overall, the ON/OFF ratio peaked at approximately −11<ΔG<−8 kcal/mol (with NUPACK in DNA mode and at 1M Na+), where the effective hybridization H′ (=H+1) was 12-14 bp. The inventors note that even at ΔG˜−5 kcal/mol, the leak activity is approximately half of the ON activity, indicating that the pulling force of RNAP may unfold the stem structure.

To overcome the pulling force of RNAP, the inventors introduced support hybridization (FIGS. 18E and F). The inventors expected that with support hybridization, the inventors could stabilize the stem structure against the RNAP pulling force while maintaining a long toehold length to obtain high miRNA affinity. The inventors introduced GC pairs at the root of the stem. With different numbers of GC pairs, a decrease in leak activity was observed for the support hybridization lengths (GC pairs) of 2 and 3, increasing the ON/OFF ratio. However, as the absolute activity in the ON state was decreased, the inventors used the sensor with 1 bp of support hybridization.

The inventors next evaluated the effective loop length L (FIGS. 18G and H). The inventors compared the chip activity of effective hybridization H′=12 bp with variable loop length L (1-21 nt). The chip activity decreased as the loop shortened. This result may be explained by two factors: shortening the effective sensor arm and lowering the affinity for miRNA. Therefore, the inventors used a sensor with 21 nt loop.

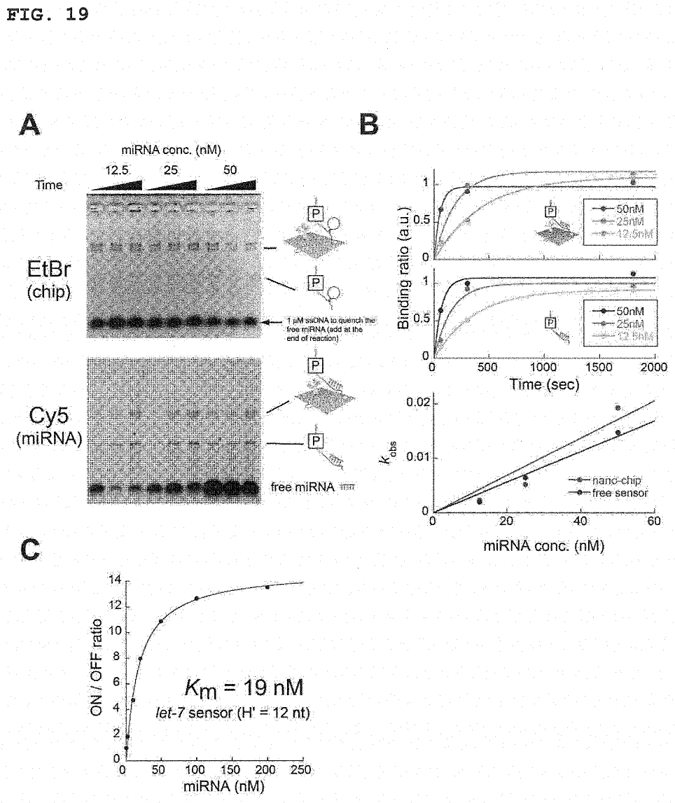

Overall, for the let-7 (this miRNA is 21 nt) sensor, the inventors generally used a sensor with H′=H+support hybridization=11+1=12 nt, T=(T+H)−H=21−H=10 nt and L′=L+T=21+10=31 nt (ΔG=−8.6 kcal/mol at 1 M Na+, and −7.7 kcal/mol at 50 mM Na+ and 18 mM Mg++ (condition of the PURE system)). Next, the inventors measured the kinetic parameters of this let-7 sensor. The inventors obtained an miRNA binding constant (kon) of ˜3×10⁵ /M/s from the gel-shift assay (FIGS. 19A and B), and an apparent Michaelis constant (Km) of ˜19 nM from activity measurements (FIG. 19C).

### <Orthogonality of the ON Switch>

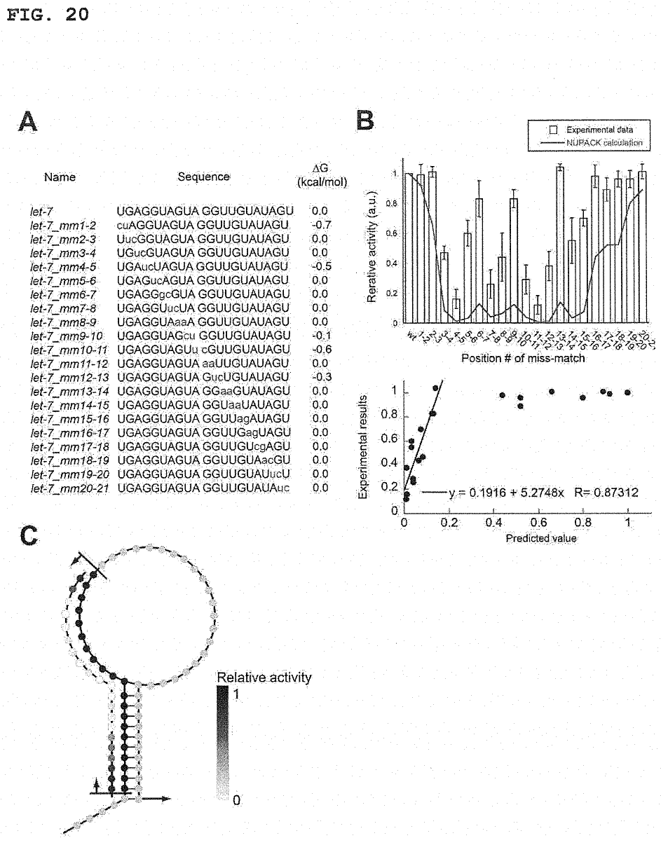

To use the sensor with genetic circuits, confirming its orthogonality to other miRNAs is important. To examine the molecular basis of the orthogonality of the sensor, the inventors introduced mismatches into the miRNA-sensor pairs (FIG. 20A). The inventors prepared 20 mismatched-miRNAs with two successive mismatches (e.g., mm1-2 had mismatches at the 1st and 2nd nucleotides) and low secondary structure formation ability (ΔG˜0). Comparing to the binding ratio calculated using NUPACK, the inventors found that the chip activity increased linearly up to a 15% predicted binding ratio and that above 15%, the mismatched miRNA activated the sensor fully (FIG. 20B). The mismatch position affecting the chip activity was located in the toehold domain and hinge region between the toehold and the stem (FIG. 20C). Conversely, mismatches located in root part of the stem did not affect the activity. These results suggested that sequence differences in-between the 1st nucleotide and 15th nucleotide were detectable, whereas those in-between the 16th nucleotide and 21st nucleotide were not (the position was numbered from the 5′ end of the miRNA).

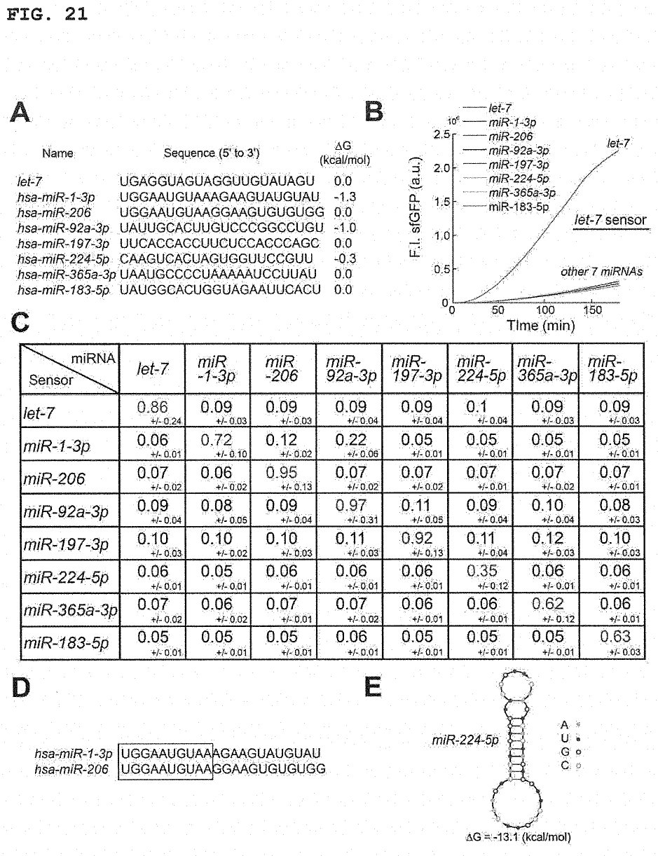

After confirming the orthogonality of the chip to the mismatched miRNAs, the inventors selected 7 HeLa cell-expressed miRNAs with low secondary structure formation ability (FIG. 21A). The inventors found that the let-7 sensor responded to only let-7 miRNA (FIG. 21B). Additionally, other sensors responded only to their cognate miRNA, demonstrating the orthogonality of the sensors (FIG. 21C). To evaluate the extent of orthogonality, the inventors paid attention to miR-1-3p, which has 10 identical sequences with miR-206 at the 5′ end (FIGS. 21C and D). As expected from the mismatched experiments (FIG. 20), the miR-1-3p sensor only slightly responds to miR-206, suggesting that the designed sensor could distinguish a similar sequence. The inventors note that, although the inventors increased the miRNA concentration from 25 to 100 nM, the response of the miR-224-5p sensor to its cognate miRNA was lower than those of the other sensors. The inventors speculated that this result is caused by the self-dimerization of miR-224-5p (FIG. 121E). Therefore, this invention's approach may not be suitable for detecting self-dimerized miRNA.

### <OFF Switch Design>

The OFF switch design was the reverse of the ON switch design, i.e., miRNA binds to the unfolded sensor structure and induces the sensor folding into the stem-loop structure with a short effective sensor arm, thereby lowering the chip activity (FIG. 22A). The OFF sensor had an anti-miRNA domain to which miRNA can hybridized, a polyT domain and, in-between these two, a supported hybridization domain (FIG. 22B). The inventors found that sensors without a supported hybridization domain exhibited a low ON/OFF ratio (FIGS. 22C and D) and that even the sensor with a supported hybridization domain showed a low ON/OFF ratio at 37° C. Therefore, the inventors obtained data from the sensors with a supported hybridization domain at 23° C. because both the divided miRNA target domain (˜11 nt for each) and the RNAP pulling force reduce the stability of hybridization between the miRNA and anti-miRNA domains of the sensor. Kinetic analysis revealed that for the sensor with a 6 bp supported hybridization domain, the inhibition constant (Ki) was 46 nM (FIG. 22E).

### <2-Input AND Switch Design (Related to FIG. 23)>

To design the 2-input AND switch, the inventors simply connected two ON switches in tandem (FIG. 23A). MicroRNA (miRNA) hybridization sufficiently increased the effective sensor arm length for activation (FIG. 23B). When the inventors connected the let-7 and miR-197-3p ON switches in tandem (FIG. 23C), the resulting AND switch was active only when both miRNAs were present (FIG. 23D). The inventors further confirmed the single chip operation of this AND switch using water-in-oil (w/o) droplet experiments (FIG. 23E).

The inventors note that the inventors used 87 nt ssDNA (=maximum length obtainable from DNA synthesis service (L_{service}. here 100 nt)−length of the primer (Lₚᵣᵢₘₑᵣ. here 13 nt)) for 2-input AND. The inventors optimized the polyT length and stem loop length of each ON switch using NUPACK to maximize the binding ratio of miRNA to the sensor. The inventors also optimized the dsDNA linker length. In the active state, the number of nucleotides was increased by 22 nt (one miRNA length) and 6 nt (=87−59−22) for dsDNA and ssDNA respectively; thus, the difference was approximately equal to 26 nt dsDNA−(22+6×0.23/0.34). The dsDNA for the ON switch was 45 nt long; thus, the inventors adjusted the dsDNA linker by approximately 19 nt (13, 19 or 25 nt), and found 19 nt to be best for let-7/miR-197-3p AND switch.

### <3-Input AND Switch Design (Related to FIG. 24)>

To design the 3-input AND switch, the inventors simply connected three ON switches in tandem (FIG. 24A). When the inventors connected the let-7, miR-206 and miR-92a-3p ON switches in tandem (FIG. 24B), the resulting AND switch was active only when all three miRNAs were present (FIG. 4C). Similar behavior was observed from 3-input AND switches for let-7/miR-206/miR197-3p and let-7/miR365a-3p/miR183 (FIG. 24C). The inventors further confirmed the single chip operation of this AND switch using water-in-oil (w/o) droplet experiments (FIG. 24D).

The detailed design approach was similar to that of the 2-input AND switch. Briefly, the inventors considered the differences 1) between the 2-input and 3-input AND switch, or 2) between ON switch and 3-input AND switch, and with 133 nt (˜44 nt×3+spacer) ssDNA linker length, the inventors adjusted the dsDNA linker length (45 nt) and inter-molecular distance (70 nm).

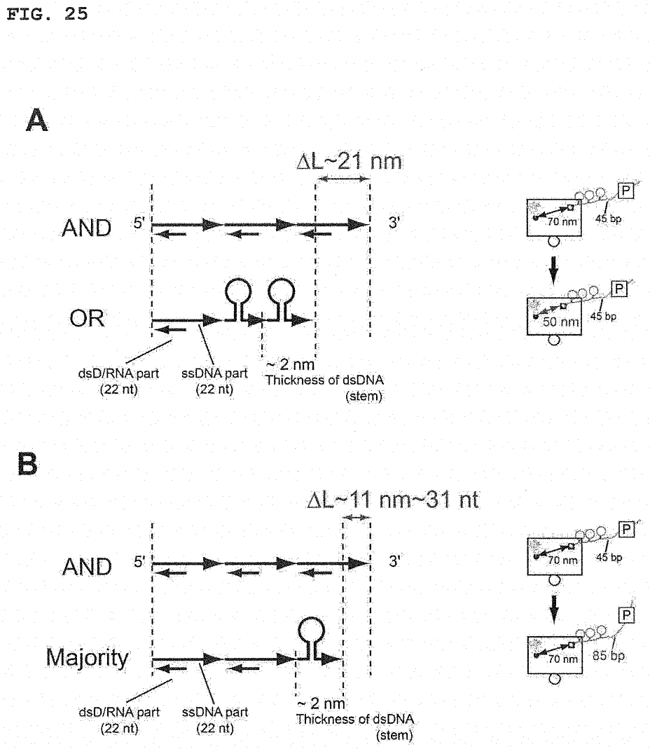

The approach of modulating intermolecular distance allowed us to change the logic function in different configurations of the nano-chip. With this approach, the 3-input “AND gate” could be converted to 3-input “OR gate” or 3-input “Majority gate” by changing the molecular layout (anchor part) or linker length, respectively, which suggests that repeatable reprogramming of the logic circuit is possible using an identical sensor sequence (FIGS. 4D, E and FIG. 25).

<Different Logic Function with Identical Gene-Substrates (Related to FIG. 25)>

With the proposed integrated method, modulation of the intermolecular distance and linker length was used to change the collision frequency of the enzyme (RNAP) and enzyme-binding domain (promoter) and the subsequent response to the environmental signal (FIG. 16E). The inventors therefore also changed the anchor part and dsDNA linker length for the 3-input AND switch (FIG. 25). The results showed the conversion to a 3-input OR switch by changing the intermolecular distance from 70 to 50 nm, and to a 3-input Majority switch by extending the dsDNA linker from 45 to 85 nt. These results suggest that reprogramming logic circuits with identical sensor sequences might be possible

The detailed design principle is as follows:

1) OR switch: In the AND switch, three simultaneous inputs are required, whereas in the OR switch, only a single input is required. Therefore, the inventors changed the intermolecular distance from 70 to 50 nm, a difference comparable to two inputs minus the thickness of two stem-structures (22 nt×2×0.34 nm/nt+22 nt×2×0.23 nm/nt−4˜21 nm. FIG. 25A).

2) Majority switch: In the Majority switch, two simultaneous inputs can activate the switch. Therefore, the inventors changed the dsDNA linker length from 45 to 85 bp, a difference comparable to one input minus the thickness of one stem-structure (22 nt×1×0.34 nm/nt+22 nt×1×0.23 nm/nt−2˜11 nm˜31 nt dsDNA. FIG. 25B).

### Example 9

### Example 9

### Construction of Genetic Circuits with Nano-Chips

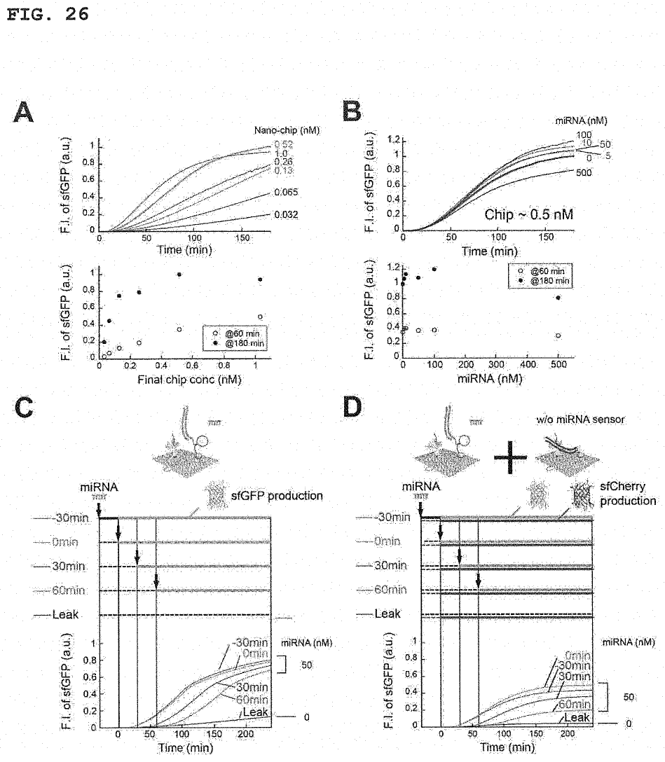

The inventors constructed genetic circuits with nano-chips. The inventors connected two “AND switches” that could respond to three miRNAs in solution (FIG. 4F, FIGS. 26-28). The first “AND switch” responds to two miRNAs, miR-206 and miR-92a-3p, and produces let-7 miRNA as a communication signal. The second “AND switch” responds to miRNA-197-3p and a communication signal. Thus, the total circuit constructed with nano-chips computes the miRNA profile and outputs the mRNA of sfGFP (FIG. 4F). Further optimization of the molecular layout, RNAP activity and gene substrate sequence should improve the switch activity, especially in suppressing the leakage of switches that might result from the scanning ability of the RNAPs (FIG. 18).

### <Cascade Reaction Using the Nano-Chip (Communication Between Chips; Related to FIG. 26-28)>

To perform a complex task, efficient cascade reactions (communication) of the logic operations are important. Accordingly, 1) high transmitted flow, 2) high orthogonality of the (input/transmitted/output) flow and 3) low leak flow are the key factors. As the inventors already considered high orthogonality (in section 3: ON switch design) and low leak flow (in the main text and section 3), the inventors focused on the 1) high transmitted flow in this working example and examined the effects of resource (e.g., energy) limitation, miRNA inhibition and a combination of both.

The inventors first examined the effect of resource limitation. As the inventors used a 1st generation integrated nano-chip, in which the cascade reaction (communication) of the logic chip was based on a reaction-diffusion system, the transmission rate depended on the output flow of the logic chip. A simple way to increase the output flow and accelerate the transmission rate is increasing the concentration of the logic chip. However, in (w/o) droplets and test tubes, whereby the metabolic system is not completely reconstituted at present, resources (e.g., energy) limitation might be a problem. For example, in the multi-step genetic circuit, the chip in the last line is activated with a substantial lag compared to the activation of the chip in the front line, and to guarantee the operation of the whole system, all chips in each step should be activated before resource depletion. The inventors therefore started to optimize the chip concentration (FIG. 26A). At a 0.52 nM chip concentration, a representative value between 0.032 and 1 nM, the inventors observed maximum activity at 180 min after the reaction initiating. By contrast, the fluorescence intensity of sfGFP was saturated at approximately 90 min at a 1 nM chip concentration, indicating resource consumption. Thus, the inventors chose concentrations of 0.5 nM or lower as the basal condition.

The inventors next measured the inhibitory effect of miRNA, the signal and the transmitter, on our genetic circuit, as some RNAs have been reported to inhibit T7 RNAP activity (FIG. 26B). At 500 nM miRNA, similar to other concentrations between 0 to 500 nM, the sfGFP fluorescence intensity (the indicator of the chip activity) was approximately 86% at 60 min and 81% at 180 min, indicating that miRNA had little effect on transcription and translation. As the inventors added several kinds of miRNA into one reaction for multiple input experiments performed later, the inventors chose 100 nM or less miRNA as the basal conditions to ensure the activation of the chips without affecting the transcription-translation system.

After optimizing the chip and miRNA concentrations, the inventors confirmed the effect of activation timing (FIG. 26C). If the idling reactions consume resources, the activity of the chip after activation may decrease. The inventors added the miRNA at −30, 0, +30 and +60 min after elevating the temperature to 37° C. and initiated the idling reactions. The inventors observed similar initial increase when adding miRNA at −30 and 0 min. This result could be explained by the kon of miRNA to the sensor (3×10⁵ /M/s, FIG. 19) because only 1 min is required for 50 nM miRNA, which the inventors used for this experiment, to bind to the sensor (50 nM×3×10⁵ /M/s=0.015 /s; thus, τ=67 s˜1 min). Additionally, the inventors found that with the addition of miRNA at +60 min, the sfGFP fluorescence intensities at 90 and 180 min after addition were 96% of that obtained when adding miRNA at −30 min, indicating that the timing of the miRNA addition had little effect. The inventors next examined the sensor-chip activity under the condition where the other chip was functioning during the idling reaction, i.e., the sensor-less nano-chip generating mCherry (sfCherry) mRNA and thus the transcription-translation system were operating in the background the entire time. At 90 min after miRNA addition, under the conditions of miRNA addition at +30 and +60 min, the activity was 91 and 60% of that of miRNA addition at −30 min, respectively, indicating that in this case, the timing of miRNA addition is important (FIG. 26D).

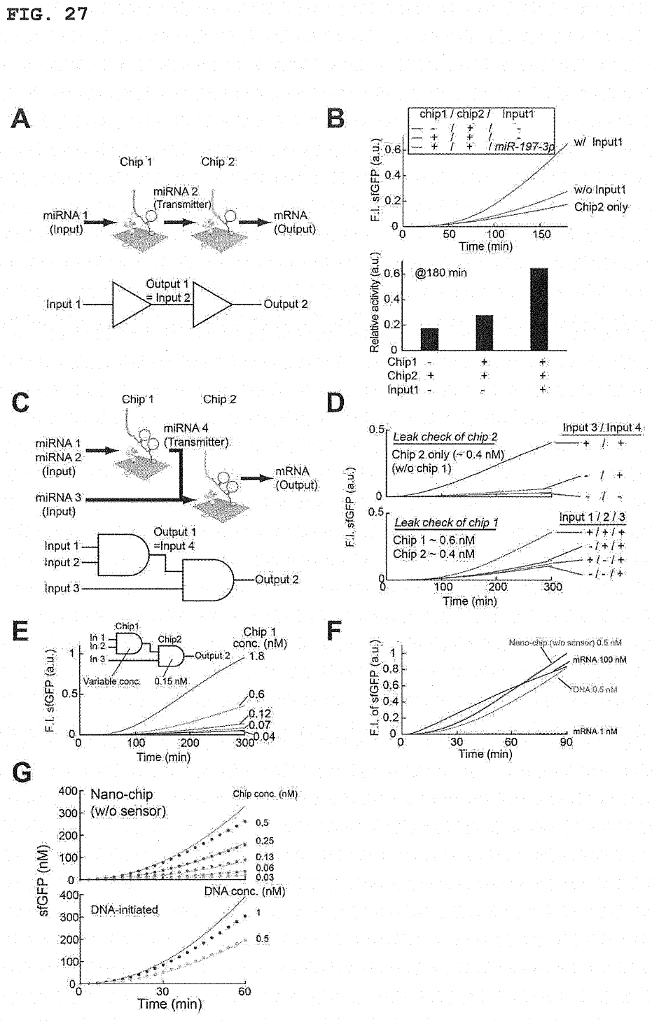

After evaluating the effects of resource limitation, miRNA inhibition and the reaction initiation point, the inventors measured the activity of the genetic cascade reaction. The inventors observed reactions that included two ON switches (FIGS. 27A and B), and two AND switches (FIGS. 27C and D). To understand the kinetics of the cascade reaction in more detail, the inventors first examined the effects of output flow on each step. At 0.04 to 1.8 nM 1st chip, the overall output, i.e., the production of sfGFP, was accelerated (FIG. 27E). To compare the effect of different gene-substrates on the kinetics of the genetic circuit, the inventors measured the gene expression activity of 1) the integrated nano-chip, and diffusion reaction-based system of 2) DNA-initiated and 3) mRNA-initiated, in a test tube (FIG. 27F). In the cell-free transcription-translation PURE system, mRNA-initiated conditions produced sfGFP the fastest; however, the efficiency was very low, and a 200 times higher concentration was required for similar sfGFP production (100 nM mRNA vs. 0.5 nM nano-chip to and DNA). Comparing the nano-chip-initiated and DNA-initiated (RNAP˜30 nM) reactions revealed that the chip-initiated reaction was faster. This result may be explained by the fact that the effective concentration on the substrate-gene of nano-chip being greater than 2 μM. To analyze the kinetics of the nano-chip based genetic circuit quantitatively, the inventors fitted the data with the Systems Biology Markup Language (SBML) simulator (FIG. 27G). The estimated RNA production rate was 0.034 and 0.02 /s/molecule of substrate-gene for nano-chip-initiated (RNAP is at the same concentration as the nano-chip, e.g., 0.5 nM in FIG. 27F) and DNA-initiated (RNAP˜30 nM; thus, 60 times greater than the nano-chip concentration in FIG. 27F) reactions, respectively. This difference, although not large under these experimental conditions, would affect the multi-step cascade reaction, particularly at low RNAP and substrate-gene concentrations. This situation would occasionally be encountered upon encapsulating the complicated genetic circuits in a confined reaction volume (e.g., (w/o) droplets/artificial cells) while preventing resource consumption.

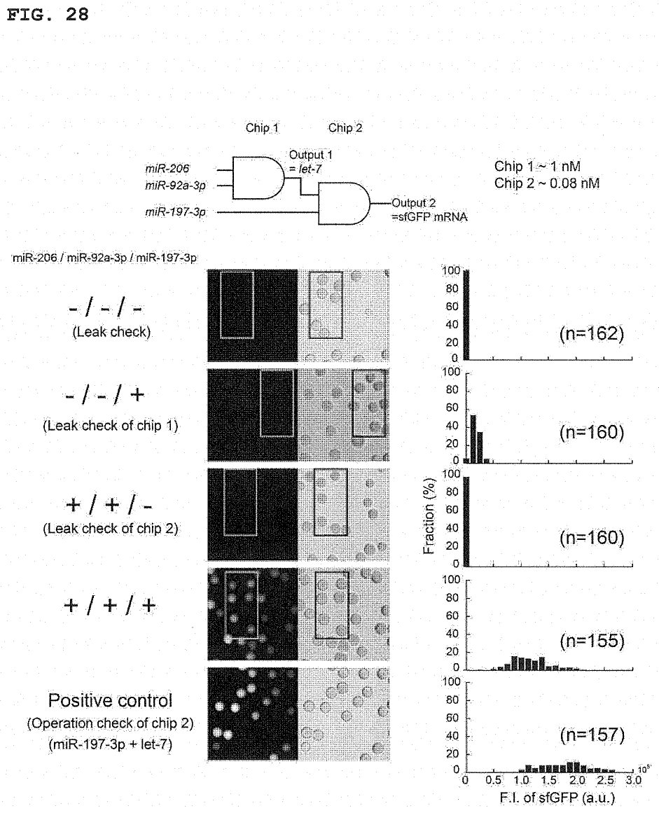

Overall, the inventors monitored a genetic circuit comprising two AND chips in water-in-oil (w/o) droplets (FIG. 28). For rapid and efficient communication, the inventors increased the first AND chip concentration to 1 nM (approximately 1000 chips/droplet) and, to reduce resource consumption by translation, decreased the second AND chip concentration to 0.08 nM (approximately 80 chips/droplet). Under these conditions, autonomous detection of the miRNA profile of the droplet was achieved.

### Example 10

### Example 10

### LacI/IPTG Sensor

When using the integrated method, rendering the sensor responsive to various signals might be possible because the sensor has no material limitations. For example, the inventors used dsDNA as the sensor material and made ON and OFF switches using a LacI-LacO pair (FIG. 29). LacI, a dsDNA binding protein that forms a tetramer, can bind to two LacO sequences (FIG. 29A). The inventors constructed LacI sensors featuring two LacO sequences upstream of the promoter sequence. As these sensors were anchored at different positions, they showed opposite functions (ON and OFF) (FIG. 29B). Although the linker for the ON switch is overly long for RNAP to bind to the promoter sequence, the linker being shortened by LacI binding affords an effective linker length suitable for transcription (FIG. 29C). Further addition of isopropyl β-D-1-thiogalactopyranoside (IPTG) induced the release of LacI from the LacO sequence, resulting in the “ON switch” being disabled (FIG. 29D). Conversely, for the OFF switch, the linker length, which is suitable for transcription in the absence of LacI, is shortened in the presence of LacI, hence shutting off the transcription activity (FIGS. 29E and F).

### Example 11

### Example 11

### Design of Ampicillin (Amp) Switch

The inventors made switch using aptamer sensor that can bind low-molecular compound. For example, using ssDNA as sensor material, the inventors made OFF switch that responded to Ampicillin (Amp, FIG. 30). Amp aptamer takes a stem-loop structure (FIG. 30A). The inventors made an Amp sensor that has three aptamer sequence in the upstream of the promoter sequence. Upon Amp binding, the reach length of the sensor arm is not enough long to fully activate the nano-chip (FIG. 30B). Under the existence of Amp (w/ Amp), the inventors observed the activity decrease of the transcription and subsequent translation.

### Example 12

### Example 12

### Design of Not Switch (NAND-Type)

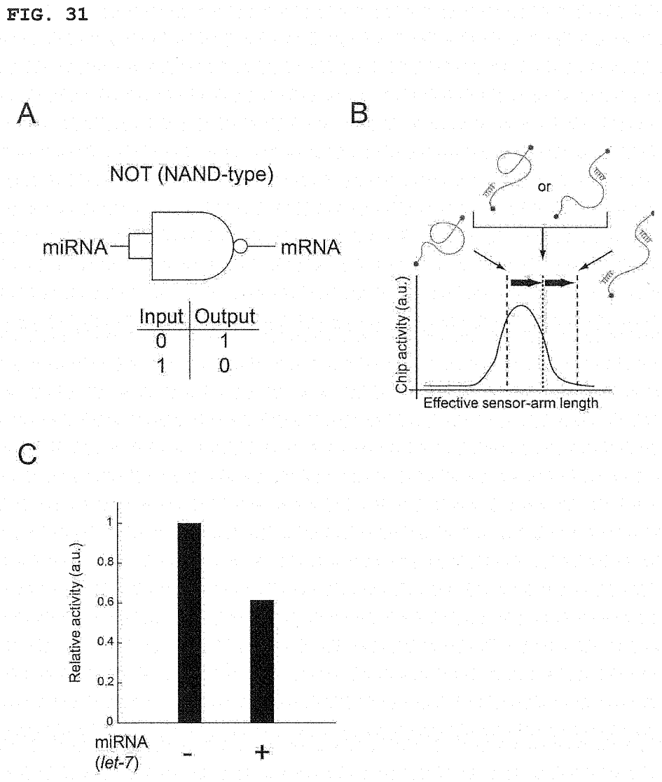

The inventors made NAND switch using miRNA sensors. To design the 2-input NAND switch, the inventors connected two ON switches in tandem (FIG. 31A). Upon hybridization with miRNAs, the reach length of the sensor arm is too long, and thus inactivate the nano-chip (FIG. 31B). Under the existence of miRNA, the inventors confirmed the decrease of activity (FIG. 31C).

### Example 13

### Example 13

### Switching and Reprogramming of Logic Function by Changing the Effective Anchor Point

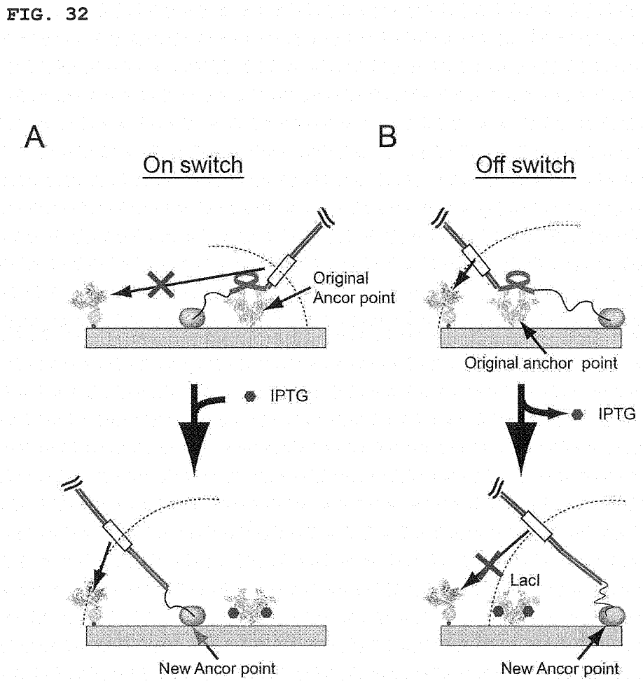

To design the switching and the reprogramming of logic function, the inventors introduced second fixed part to the base member, which can bind to the sensor. For example, the inventors used dsDNA as the sensor material and fixed the LacI as the second fixed part to the base member, making ON and OFF switches using a LacI-LacO pair (FIG. 32). Upon IPTG binding to the initial fixed part (the second fixed part: LacI), the reach length of the sensor arm is lengthened, activating the nano-chip (FIG. 32A). Or upon IPTG binding to the initial anchor point (the second fixed part: LacI), the reach length of the sensor arm is shortened, shutting off the nano-chip activity (FIG. 32B).

These results suggested the potential of the integrated method for detecting various signals through logic functions enabled by modulating the effective sensor arm length. Furthermore, sensors with identical attached substrate-genes demonstrated opposite logic functions, suggesting future repeatable reprogramming of the genetic circuit through effective linker length modulation, sensor layout change and scaffold transformation.

### Example 14

### Example 14

### ON/OFF Switching by UV Irradiation

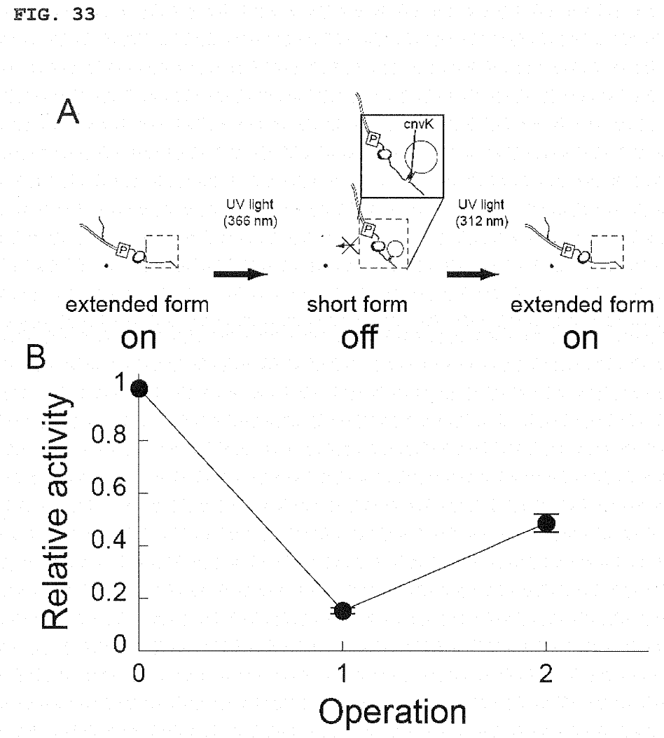

Using the cross-linking and reverse reaction of photoreactive cross-linking nucleotide (cnvK), the inventors designed the chip that can photoswitch the nano-chip activity between ON and OFF state. 5 min irradiation of 366 nm UV light converted the cnvK UV sensor to the short form, resulting in the switching off of the nano-chip activity (operation #1, FIG. 33A). Further 10 min irradiation of 312 nm UV light resumed the nano-chip activity (operation #2, FIG. 33A).

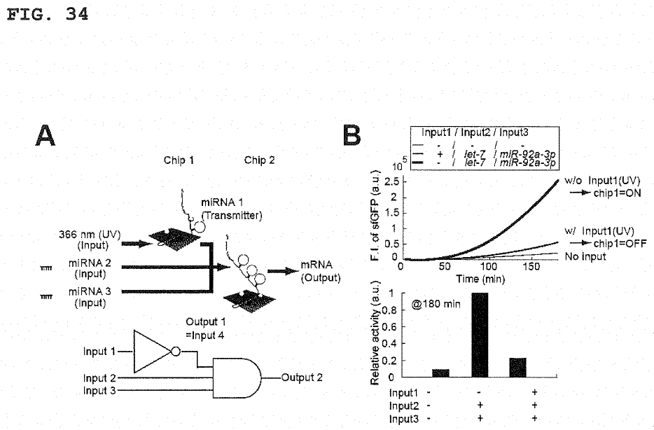

FIG. 34B clearly shows that it is possible to design the ON/OFF switching device with this invention.

### Example 15

### Example 15

### Construction of Genetic Circuits Responding to UV Irradiation and miRNA Profile

The inventors constructed the genetic circuits using device1 (chip1) having UV sensor and device2 (chip2) having miRNA sensor. The inventors connected two “NOT switch” that could respond to UV and “AND switch” that could respond to three miRNAs in solution (FIG. 34A). In the circuit, in the absence of UV irradiation (365 nm), chip 1 produces the communication transmitter miRNA (miR-206). Chip 2 produces the sfGFP mRNA only when all of let-7, miR-92a-3p and transmitter miRNA (miR-206) exist. Thus, the whole circuit compute the UV irradiation and miRNA profile, and output sfGFP mRNA (FIG. 34A).

In the genetic circuit, the fluorescent intensity of sfGFP showed time-dependent change upon UV irradiation and miRNA profile. Therefore, with the invented device, it is possible to construct genetic circuits that contain the logic circuit executing various logic operation (FIG. 34B).

### Example 16

### Example 16

### Confirmation of Nano-Chip Function In Vivo/Individual System (Living Organism)

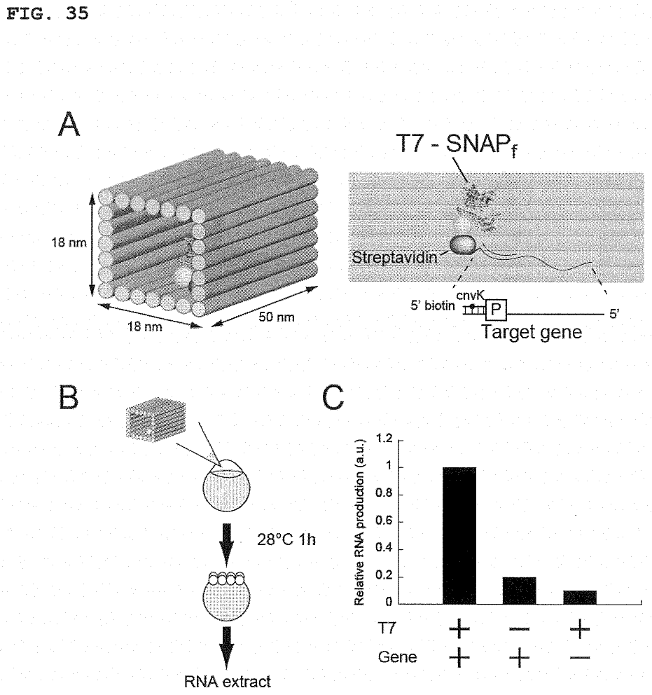

Nano-chip (cage) was microinjected into the zebrafish embryo, and the RNA production was confirmed by real-time qRT-PCR machine (FIG. 35A-C). Nano-chip (cage) was microinjected into the zebrafish embryo, and the RNA production was confirmed by real-time qRT-PCR machine (FIG. 35A-C). Thus, the gene expression device of the present invention is provides the method to control the gene expression in vivo/in individual system (living organism, FIG. 35C).

### INDUSTRIAL APPLICABILITY

The device for genetic circuits of the present invention and the genetic circuits containing the device provide a device for genetic circuits that is under extremely reduced restrictions on the materials of a sensor sensing the environment, capable of performing controlled gene expression with sufficiently suppressed crosstalk. Therefore, since the present invention can be applied to various applications such as, but are not limited to, the treatment of gene diseases, biomarker detection, and efficient synthesis and the like, and the present invention is industrially extremely useful.

This application is based on patent applications No. 2017-232193 filed in Japan on Dec. 1, 2017 and No. 2018-135358 filed in Japan on Jul. 18, 2018, the contents of which are incorporated in full herein by reference.

1. A device for a gene circuit, comprising an open-close mechanism constituted of a catalyst, a target gene, and a shape change element, wherein
the catalyst induces an expression of the target gene by contacting the target gene,
the shape change element has a first site and a second site, and has a structure in which a distance between the first site and the second site changes by an action of an activation source, and
the open-close mechanism has the shape change element as a movable part, and is configured to perform an open-close motion in which the target gene and the catalyst relatively approach and contact with each other or relatively leave the contact state in response to a change in the distance between the first site and the second site in the shape change element.

2. The device according to claim 1, further comprising a base member,
either one of the catalyst and target gene is fixed on the base member, and the other is fixed on the side of the first site of the shape change element in the movable part,
the side of the second site of the shape change element in the movable part is fixed on the base member,
the open-close mechanism is configured such that the catalyst and the target gene relatively approach and contact with each other or relatively leave the contact state by an increase in the distance between the first site and the second site in the shape change element, and
the open-close mechanism is configured such that the catalyst and the target gene relatively approach and contact with each other or relatively leave the contact state by a decrease in the distance between the first site and the second site in the shape change element.

3. The device according to claim 1, further comprising a base member, a first shape change element as a mutually independent first movable part, and a second shape change element as a mutually independent second movable part,
the catalyst is fixed on the side of a first site of the first shape change element in the first movable part,
the target gene is fixed on a first site side of the second shape change element in the second movable part,
the second site side of each shape change element in the first and the second movable parts is fixed on the base member,
the open-close mechanism is configured such that the catalyst and the target gene relatively approach and contact with each other or relatively leave the contact state by an increase in the distance between the first site and the second site in each shape change element, and
the open-close mechanism is configured such that the catalyst and the target gene relatively approach and contact with each other or relatively leave the contact state by a decrease in the distance between the first site and the second site in each shape change element.

4. The device according to claim 1, further comprising a base member having flexibility, wherein
the catalyst and the target gene are placed on the base member, the first site and the second site of the shape change element are fixed on the base member, and the open-close mechanism is configured such that a decrease or an increase in the distance between the first site and the second site deforms the base member, and the catalyst and the target gene relatively approach and contact with each other or relatively leave the contact state by the deformation of the base member.

5. The device described in claim 1, wherein the movable part has shape change elements in the number of n which are connected to each other, wherein n is an integer of 2 or more, and the movable part is configured such that the target gene and the catalyst relatively approach and contact with each other or relatively leave the contact state when the distance between the first site and the second site changes in at least one of the shape change elements among the shape change elements in the number of n.

6. The device according to claim 5, wherein the device is configured such that the target gene and the catalyst relatively approach and contact with each other or relatively leave the contact state when the distance between the first site and the second site in all of the shape change elements in the number of n changes.

7. The device described in claim 1, wherein the movable part has shape change elements in the number of n which are connected to each other, wherein n is an integer of 3 or more, and the movable part is configured such that the target gene and the catalyst relatively approach and contact with each other or relatively leave the contact state when the distance between the first site and the second site changes in not less than half of the shape change elements among the shape change elements in the number of n.

8. The device according to claim 2, wherein either one of the catalyst and target gene is fixed on a first anchor part on the base member, and the other is fixed on a first fixed part on the side of the first site of the shape change element in the movable part,
a second fixed part on the side of the second site of the shape change element in the movable part is fixed on a second anchor part on the base member,
a third fixed part is provided between the first fixed part and the second fixed part of the movable part, a third anchor part is provided on the base member at a position where the third fixed part of the movable part can reach, and
the third anchor part and the third fixed part are configured to be bondable to each other.

9. The device according to claim 8, wherein the third anchor part is located between the first anchor part and the second anchor part.

10. The device according to claim 8, wherein the third anchor part and the third fixed part are bondable to each other by an action of an activation source for bonding.

11. The device according to claim 8, wherein the third anchor part and the third fixed part are separable from each other by an action of an activation source for release when the third fixed part is bonded to the third anchor part.

12. The device described in claim 8, wherein the movable part has a plurality of shape change elements which are connected, and the third fixed part is located between the plurality of the shape change elements.

13. The device according to claim 2, wherein either one of the catalyst and target gene is fixed on a first anchor part of the base member, and the other is fixed on a first fixed part on the side of the first site of the shape change element in the movable part,
a second fixed part on the side of the second site of the shape change element in the movable part is fixed on a second anchor part on the base member,
a third anchor part is provided on the base member, a third fixed part is provided between the first fixed part and the second fixed part of the movable part,
the third anchor part and the third fixed part are bonded to each other such that they are separable from each other by an action of an activation source for release,
the position of the third anchor part on the base member is selected such that the first fixed part does not reach the first anchor part, and the first fixed part is configured to reach the first anchor part when the third anchor part and the third fixed part are separated from each other by an action of the activation source for release, whereby the target gene and the catalyst relatively approach and contact with each other.

14. The device according to claim 2, wherein either one of the catalyst and target gene is fixed on a first anchor part of the base member, and the other is fixed on a first fixed part on the side of the first site of the shape change element in the movable part,
a second fixed part on the side of the second site of the shape change element in the movable part is fixed on a second anchor part on the base member,
a third anchor part is provided on the base member, a third fixed part is provided between the first fixed part and the second fixed part of the movable part,
the third anchor part and the third fixed part are bonded to each other such that they are optionally separated from each other by an action of an activation source for release,
the position of the third anchor part on the base member is selected such that the distance between the first anchor part and the third anchor part is equal to or longer than the distance between the first fixed part and the third fixed part, whereby the target gene and the catalyst come into contact.

15. The device according to claim 14, wherein the distance between the first fixed part and the second fixed part is shorter or longer than the distance between the first anchor part and the second anchor part by a change in the distance between the first site and the second site of the shape change element when the third anchor part and the third fixed part are separated from each other by an action of the activation source for release.

16. The device described in claim 2, wherein the base member is a nanostructure composed of DNA, RNA, artificial nucleic acid, peptide, protein, or polymer, or a combination thereof.

17. The device according to claim 1, wherein the catalyst is RNA polymerase, DNA polymerase, artificial nucleic acid polymerase, or polymer synthase, the shape change element is nucleic acid, artificial nucleic acid, or polymer, and the activation source is miRNA, RNA, DNA, artificial nucleic acid, polymer LacI protein, IPTG (Isopropyl β-D-1-thiogalactopyranoside) compound, Ampicillin antibiotic, or light.

18. A gene circuit comprising one or more devices according to claim 1, and having a logic circuit configured to perform a logical operation by the open-close mechanism of the one or more devices.

19. The gene circuit according to claim 18, having one or both of a logic circuit of the following (I) and a logic circuit of the following (II):
(I) a logic circuit configured by the one or more devices and having one or more switches selected from ON switch, OFF switch, ON/OFF selector switch, AND switch, OR switch, NAND switch, NOR switch, and MAJORITY switch,
(II) a logic circuit configured by the one or more devices and having two or more switches selected from ON switch, OFF switch, ON/OFF selector switch, AND switch, OR switch, NAND switch, NOR switch, and MAJORITY switch.
